SEQUENCE LISTING (22 sequences)

SEQ ID NO: 1 - protein, 17 aa, synthetic construct
  REGION 1..17: note = Synthetic
EEEEEEEEEGGGQARGG

SEQ ID NO: 2 - protein, 5 aa, synthetic construct
  REGION 1..5: note = Synthetic
QARGG

SEQ ID NO: 3 - protein, 18 aa, synthetic construct
  REGION 1..18: note = Synthetic
EEEEEEEEEGGGRAEGGG

SEQ ID NO: 4 - protein, 15 aa, synthetic construct
  REGION 1..15: note = Synthetic
EEEEEEEEEGGGQAR

SEQ ID NO: 5 - protein, 5 aa, synthetic construct
  REGION 1..5: note = Synthetic
KLVFF

SEQ ID NO: 6 - protein, 12 aa, synthetic construct
  REGION 1..12: note = Synthetic
SAVLQSGFRKMA

SEQ ID NO: 7 - protein, 6 aa, synthetic construct
  REGION 1..6: note = Synthetic
RRRRRR

SEQ ID NO: 8 - protein, 14 aa, synthetic construct
  REGION 1..14: note = Synthetic
CGKLVFFGTSAVLQ

SEQ ID NO: 9 - protein, 5 aa, synthetic construct
  REGION 1..5: note = Synthetic
SAVLQ

SEQ ID NO: 10 - protein, 7 aa, synthetic construct
  REGION 1..7: note = Synthetic
SGFRKMA

SEQ ID NO: 11 - protein, 9 aa, synthetic construct
  REGION 1..9: note = Synthetic
CGAVLQDDD

SEQ ID NO: 12 - protein, 10 aa, synthetic construct
  REGION 1..10: note = Synthetic
AVLQFFVLKC

SEQ ID NO: 13 - protein, 17 aa, synthetic construct
  REGION 1..17: note = Synthetic
RVRRSAVLQSGFRKMAC

SEQ ID NO: 14 - protein, 22 aa, synthetic construct
  REGION 1..22: note = Synthetic
CGKLVFFGTSAVLQSGFRGDDD

SEQ ID NO: 15 - protein, 8 aa, synthetic construct
  REGION 1..8: note = Synthetic
SGFRGDDD

SEQ ID NO: 16 - protein, 20 aa, synthetic construct
  REGION 1..20: note = Synthetic
  MOD_RES 1: note = Propargylglycine (Pra)
GSAVLQSGFRKMAGRRRRRR

SEQ ID NO: 17 - protein, 24 aa, synthetic construct
  REGION 1..24: note = Synthetic
  MOD_RES 1: note = Propargylglycine (Pra)
KLVFFGSAVLQSGFRKMARRRRRR

SEQ ID NO: 18 - protein, 29 aa, synthetic construct
  REGION 1..29: note = Synthetic
  MOD_RES 1: note = Propargylglycine (Pra)
KLVFFGGGSAVLQSGFRKMAGGGRRRRRR

SEQ ID NO: 19 - protein, 306 aa, synthetic construct [sequence omitted]

SEQ ID NO: 20 - protein, 4 aa, synthetic construct
  REGION 1..4: note = Synthetic
AVLQ

SEQ ID NO: 21 - protein, 9 aa, synthetic construct
  REGION 1..9: note = Synthetic
RVRRSAVLQ

SEQ ID NO: 22 - protein, 8 aa, synthetic construct
  REGION 1..8: note = Synthetic
SGFRKMAC

(19) United States
(12) Patent Application Publication
(10) Pub. No.: US 2025/0361277 A1
(43) Pub. Date: Nov. 27, 2025

(54) PROTEASE-RESPONSIVE SURFACE-POTENTIAL-TUNABLE PEPTIDE CONSTRUCTS FOR SELECTIVE IMAGING AND ACCURATE INHIBITOR SCREENING

(71) Applicant: The Regents of the University of California, Oakland, CA (US)

(72) Inventors: Jesse JOKERST, San Diego, CA (US); Yong CHENG, San Diego, CA (US)

(21) Appl. No.: 18/672,926

(22) Filed: May 23, 2024

(51) Int. Cl. C07K 14/47; A61K 38/00; A61K 47/64; A61K 47/65; A61K 49/00; A61P 31/14

(52) U.S. Cl. CPC C07K 14/47; A61K 47/6455; A61K 47/65; A61K 49/0021; A61K 49/0056; A61P 31/14; A61K 38/00

(57) ABSTRACT

In alternative embodiments, provided is a protease-responsive and surface-potential-tunable peptide-conjugated AIEgens (EGTP) for TMPRSS2 selective imaging and accurate inhibitor screening, where EGTP comprises four segments: the first is a polyglutamic acid (Glu, E for short in EGTP) that increases the solubility, blocks the positive charges and cell-penetrating ability of PyTPE; the second comprises a spacer trimylglycine (GGG, G) designed to enhance probe flexibility and reduce steric hindrance for TMPRSS2-substrate interactions; the third component second comprises a TMPRSS2-responsive peptide (QAR, T), which can be cleaved by TMPRSS2 after QAR sequence; and the fourth second comprises a positive charged AIEgens (PyTPE, P). In alternative embodiments, provided are main protease (Mpro)-responsive and modular-peptide-conjugated probes for the selective imaging and inhibition of SARS-CoV-2 infected cells via enzyme-instructed self-assembly and aggregation-induced emission.

### STATEMENT AS TO FEDERALLY SPONSORED RESEARCH

This invention was made with government support under DE031114, AI157957, and AG065776-01S1 awarded by the National Institutes of Health (NIH). The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on May 23, 2024, is named “0321.152471.xml” and is 29,587 bytes in size. The sequence listing contained in this .XML file is part of the specification and is hereby incorporated by reference herein in its entirety.

### TECHNICAL FIELD

This invention generally relates to infectious diseases and immunoassays. In alternative embodiments, provided is a protease-responsive and surface-potential-tunable peptide-conjugated AIEgens (EGTP) for Transmembrane protease serine 2 (TMPRSS2) selective imaging and accurate inhibitor screening, where EGTP comprises four segments: the first is a polyglutamic acid (Glu, E for short in EGTP) that increases the solubility, blocks the positive charges and cell-penetrating ability of PyTPE; the second comprises a spacer trimylglycine (GGG, G) designed to enhance probe flexibility and reduce steric hindrance for TMPRSS2-substrate interactions; the third component second comprises a TMPRSS2-responsive peptide (QAR, T), which can be cleaved by TMPRSS2 after QAR sequence; and the fourth second comprises a positive charged AIEgens (PyTPE, P). In alternative embodiments, provided are main protease (Mpro)-responsive and modular-peptide-conjugated probes for the selective imaging and inhibition of SARS-CoV-2 infected cells via enzyme-instructed self-assembly and aggregation-induced emission.

### BACKGROUND

Transmembrane protease serine 2 (TMPRSS2) is an extracellular protease and highly expressed by epithelial cells of nasal, lung, colon, gallbladder, kidney, and prostate that plays key roles in tissue homeostasis.¹⁻⁵ TMPRSS2 mainly consists of a transmembrane domain, a linker region, and a canonical serine protease domain with both proteolytic activity and signal transduction.⁶⁻⁹ Especially, TMPRSS2 can cleave peptide sequence after QAR.¹⁰⁻¹¹ Aberrant expression of TMPRSS2 has gained interest owing to its closely related to severe disease such as cancer, suggesting the potential as biomarker and prossible target for cancer theranostics. Moreover, coronaviruses and influenza viruses are heavily dependent on TMPRSS2 for viral activation and cell entry.¹²⁻¹⁸ TMPRSS2 inhibitors can reduce prostate cancer cell invasion and metastasis as well as partially prevent the entry of SARS-CoV-2 into the lung epithelial cells.¹⁹⁻²⁴ Monitoring TMPRSS2 activity has attracted broad attention due to its important value in imaging of cancer cells, infected cells, and identification of inhibitors,²⁵⁻²⁷ which leads to the development of fluorogenic peptide substrates, immunofluorescence antibodies, and fluorescent probes.²⁸⁻³² TMPRSS2 selective imaging and accurate TMPRSS2 inhibitor screening can be benefit not only to cancer therapy but also to against COVID-19.

Numerous efficient methods have been developed for accurate protease detection, including electrochemical analysis, acoustic or magnetic scanning platform, and optical imaging system.³³⁻³⁸ Tsien and co-workers have demonstrated a series of smart probes to evaluate protease activity with polycationic peptides, polyanionic peptides, and contrast agent.³⁹⁻⁴⁰ The positive charged cell-penetrating peptides is initially neutralized and hard to attach to the cells by polyanionic sequences. After being cleaved by targeted protease, the polycationic peptide linked probes can bind to and enter into cells for specific protease imaging. Taking the sensitivity, spatio-temporal resolution, and operation convenience into consideration, fluorescence imaging has been widely used because of its high sensitivity, excellent resolution, multicolor labeling and rapid signal acquisition.⁴¹⁻⁴⁴ There are several signal conversion mechanisms to design enzymatic fluorescent probes, likes intramolecular charge transfer, photon-induced electron transfer, Forster resonance energy transfer, and aggregation-induced emission (AIE).⁴⁵⁻⁴⁸ Among them, the fluorescence intensity of AIE luminogens (AIEgens) responds to changes in restriction of intramolecular motion with polarity or molecular-rotation-limited environment.⁴⁹⁻⁵¹ Propeller-shaped AIEgens have been widely applied to optical devices, luminescent sensors, imaging probes, and cancer theranostic.⁵² Especially, the typical AIEgen azide-functionalized tetraphenylethene pyridinium with (PyTPE) with bright yellow fluorescence mitochondrial targeting, and superior photostability was beneficial to real-time long-term cell imaging.⁵¹⁻⁵⁵ The fluorescent intensity of PyTPE can be enhanced after the cleavage of customized peptides, nucleic acids, and glycans with proteases.⁵⁶⁻⁵⁷

Transmembrane protease serine 2 (TMPRSS2) is an extracellular protease to activate both the spike protein of coronaviruses for cell entry and oncogenic signaling pathways for tumor progression. TMPRSS2 inhibition not only can effectively reduce cancer invasion and metastasis as well as partially prevent the entry of SARS-CoV-2 into host cells. There is an urgent need for both real-time tracking of TMPRSS2 expression and precise screening of TMPRSS2 inhibitors to cure cancer and ultimately prevent viral transmission.

### SUMMARY

In alternative embodiments, provided are products of manufacture, or a series of smart probes, to evaluate protease activity with polycationic peptides, polyanionic peptides, and contrast agents. The positive-charged cell-penetrating peptides are initially neutralized and hard to attach to the cells by polyanionic sequences. After being cleaved by a targeted protease, the polycationic peptide-linked probes can bind to and enter into cells for specific protease imaging.

In alternative embodiments, provided are products of manufacture, or synthetic peptide or polypeptide, comprising:
(a) a protease-responsive and surface-potential-tunable peptide-conjugated AIEgens (EGTP) for transmembrane protease serine 2 (TMPRSS2) selective imaging and accurate inhibitor screening, where EGTP comprises four segments: the first is a polyglutamic acid (Glu, E for short in EGTP) that increases the solubility, blocks the positive charges and cell-penetrating ability of PyTPE;
(b) a spacer trimylglycine (GGG, G) designed to enhance probe flexibility and reduce steric hindrance for TMPRSS2-substrate interactions;
(c) a TMPRSS2-responsive peptide (QAR, T), which can be cleaved by TMPRSS2 after QAR sequence; and
(d) a positive charged AIEgens (PyTPE, P).

In alternative embodiments, provided are products of manufacture, or synthetic PSGMR, or (Pra)KLVFFGGGSAVLQ/SGFRKMAGGGRRRRRR (where (Pra) is Propargylglycine) (the Mpro-responsive modular (self-assembling) peptides or polypeptides), comprising:
(a) an AIEgen (PyTPE, P for short in PSGMR). PyTPE has bright yellow fluorescence, excellent biocompatibility, and good photostability;
(b) a self-assembling peptide (KLVFF (SEQ ID NO:5), S) β-sheet-forming peptide derived from A-amyloid protein, that can spontaneously self-assemble into amyloid fibrils through π-π stacking, hydrogen bonding, and hydrophobic interactions;
(c) a spacer trimylglycine (GGG, G) to enhance flexibility and reduce steric hindrance for main protease (Mpro)-substrate interactions;
(d) a main protease (Mpro)-responsive peptide (SAVLQ/SGFRKMA (SEQ ID NO:6), M), and
(e) a positive hexamolyarginine (RRRRRR (SEQ ID NO:7), R) that increases both the solubility and cell-penetrating ability of PSGMR,
wherein (a) to (e) are covalently coupled,
wherein optionally (a) to (e) are covalently coupled through a Fmoc-based solid-phase peptide synthesis and a copper-catalyzed azide-alkyne click reaction,
and optionally the synthetic PSGMR forms loose nanoparticles due to the positive hexamolyarginine residues on the surface and hydrophobic core of PyTPE,
and optionally after being cleaved by main protease (Mpro), however, the hydrophilic hexamolyarginine is separated from PSG, and the self-assembling peptides with one negative charge is exposed to the nanoparticle surface, resulting in increasing self-assembly and electrostatic attraction as well as the decreasing hydrophilicity leading to PSG aggregation and nanofiber formation with strong yellow fluorescence

In alternative embodiments, provided are nanofibers comprising a plurality of synthetic PSGMR (the main protease (Mpro)-responsive modular (self-assembling) peptides or polypeptides as set forth herein.

In alternative embodiments, provided are pharmaceutical compositions comprising a nanofiber as set forth herein.

In alternative embodiments, provided are methods for selectively inhibiting the growth of a viral-infected (optionally SARS-CoV-2-infected) cell, or treating or preventing virus replication, optionally intracellular virus replication, or treating or preventing a viral infection in an individual in need thereof, comprising exposing the viral-infected infected cell or the virus to a nanofiber as set forth herein, or administering to an individual in need thereof a nanofiber as set forth herein, or a pharmaceutical composition as set forth herein.

In alternative embodiments, provided are uses of a nanofiber as set forth herein, or a pharmaceutical composition as set forth herein, for selectively inhibiting the growth of a viral-infected (optionally SARS-CoV-2-infected) cell, or preventing virus replication, optionally intracellular virus replication, or treating or preventing a viral infection in an individual in need thereof.

In alternative embodiments, provided are nanofibers or pharmaceutical compositions for use in selectively inhibiting the growth of a viral-infected (optionally SARS-CoV-2-infected) cell, or treating or preventing virus replication, optionally intracellular virus replication, or for use in treating or preventing a viral infection in an individual in need thereof, comprising exposing the viral-infected infected cell or the virus to a nanofiber as provided herein, or administering to an individual in need thereof a nanofiber as set forth herein, or a pharmaceutical composition as set forth herein.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-H illustrate structural characterization of exemplary surface-potential-tunable peptide-conjugated AIEgens (EGTP) and its derivatives:

FIG. 1A illustrates a table describing characteristics of: the dye PyTPE (TetraPhenylEthene Pyridinium); the main probe named EGTP, with Propargylglycine (Pra) linking the peptide (EEEEEEEEEGGGQARGG (SEQ ID NO:1)) to the dye PyTPE; a segment of the main probe, which is called “TGP”, or the peptide QARGG (SEQ ID NO:2) linked by Propargylglycine (Pra) to the dye PyTPE; and, the uncleavable control probe named “EGEP”, comprising the peptide (EEEEEEEEEGGGRAEGGG (SEQ ID NO:3)) linked by Propargylglycine (Pra) to the dye PyTPE, the (red) slash represents the TMPRSS2 cleavage position, the (red) superscript represents the number of charge in the probes;

FIG. 1B illustrates a high-performance liquid chromatography (HPLC) results (normalized intensity) of: blank control, EGT (EEEEEEEEEGGGQAR (SEQ ID NO:4)), PyTPE (TetraPhenylEthene Pyridinium), and surface-potential-tunable peptide-conjugated AIEgens (EGTP) incubation with Transmembrane protease serine 2 (TMPRSS2) under the 254 nm or 405 nm, over time;

FIG. 1C-E illustrate images of electrospray ionization mass spectrometry (ESI-MS) results of EGT (FIG. 1C), EGTP (FIG. 1D), and EGTP (FIG. 1E) incubation with TMPRSS2;

FIG. 1F graphically illustrates hydrodynamic sizes for EGTP and EGTP incubation with TMPRSS2, with intensity as a function of size in nm;

FIG. 1G graphically illustrates zeta potential values for PyTPE, EGTP and EGTP incubation with TMPRSS2; and

FIG. 1H illusrates images of photograph (upper image) and transmission electron microscope (TEM) images (lower images) of PyTPE and EGTP without and with TMPRSS2, in the upper panel vials contained 1, 5, 10, 20, 50, 100, 200, and 400 mM EGTP solutions, where 20 μM of PyTPE, EGTP, TGP, and EGEP were dissolved in Tris-HCl buffer with 1% DMSO; in the lower images, the right image shows and EGTP with TMPRSS2 (the insert bar is scale bar of 100 nanometer),
as discussed in further detail in Example 1, below.

FIG. 2A-H illustrate the photophysical properties of EGTP and its derivatives:

FIG. 2A-B graphically illustrate absorption (FIG. 2A) and fluorescence (FIG. 2B) spectra of EGTP, TGP and EGEP, the data showing the solubility enhancement with the decreased fluorescence intensity;

FIG. 2C-E graphically illustrate Fluorescence spectra, with relative fluorescence units (RFU) as a function of wavelengths in nm (FIG. 2C-D) (TMPRSS2 100 nM and varying amounts of EGTP, FIG. 2C) (EGTP 20 nM and varying amounts of TMPRSS2, FIG. 2D) and kinetics (RFU as a function of time) (FIG. 2E) of EGTP with different concentration of TMPRSS2 and 100 nM camostat (a serine protease inhibitor) at 590 nm showed the fluorescence increase because of TMPRSS2;

FIG. 2F graphically illutrates probe specificity of EGTP with 200 nM different proteins including TMPRSS2, bovine serum albumin (BSA), hemoglobin (HGB), main protease (M^{pro}), papain-like protease (PL^{pro}), thrombin (TB), and trypsin; and

FIG. 2G-H graphically illustrate the impact of protease inhibitors as studied with EGTP, TMPRSS2, camostat, GC376, and GRL0617; in FIG. 2H I₀ is the fluorescence intensity of EGTP, It is the fluorescence intensity of EGTP after incubation with TMPRSS2, I_{c} is the fluorescence intensity of EGTP after incubation TMPRSS2, with different concentrations of camostat,
as discussed in further detail in Example 1, below.

FIG. 3A-I illustrate TMPRSS2 selective imaging in cancer cells with EGTP:

FIG. 3A illustrates an exemplary experimental scheme of TMPRSS2 slelective imaging between TMPRSS2 low and high expression cells incubation with EGTP;

FIG. 3B-G illustrate images of confocal laser scanning microscopy (CLSM) of (FIG. 3B) HeLa cells with PyTPE and MTG, and (FIG. 3D) A549 cells, (FIG. 3E) HeLa cells, (FIG. 3F) MCF-7-GFP cellsw, and (FIG. 3G) wMCF-7-GFP and A549 co-cultured cells with EGTP (the (pink) arrows show clear fluorescence only from EGTP; and FIG. 3G the white and red dotted rings denote the MCF-7-GFP cells and A549 cells, respectively);

FIG. 3C graphically illustrates the average fluorescence intensities of A549 cells, HeLa cells, and MCF-7-GFP cells with Alexa 647 labeled TMPRSS2 antibodies; and

FIG. 3H-I graphically illustrate data for yellow fluorescence (FIG. 3H) and (FIG. 3I) green fluorescence in FIG. 3B, FIG. 3D, FIG. 3E, FIG. 3f, and FIG. 3G; the MCF-7-FGP cells are activated in the green channel, and the EGTP are activated in the yellow channel when cells express TMPRSS2,
as discussed in further detail in Example 1, below.

FIG. 4A-E illustrate data validating EGTP in the Vero cells and TMPRSS2-Vero cells:

FIG. 4A schematically illustrates an exemplary experimental scheme of Vero cells and TMPRSS2-Vero cells after incubation with different AIEgens, the enlarged image to the right shows the imaging mechanism of immunofluorescence imaging; and

FIG. 4B-E illustrate confocal laser scanning microscopy (CLSM) images and (graphically, to the right of the images) the relative fluorescence intensities of the Vero cells with (FIG. 4B) PyTPE and (FIG. 4D) EGTP incubation, and TMPRSS2-Vero cells with (FIG. 4C) PyTPE and (FIG. 4E) EGTP incubation, where the EGTP and Alexa 488 are activated in the yellow and green channel when cells express TMPRSS2,
as discussed in further detail in Example 1, below.

FIG. 5A-E illustrate data showing accurate TMPRSS2 inhibitor screening with EGTP and Camostat:

FIG. 5A schematically illustrates an exemplary experimental scheme of TMPRSS2-Vero cells after incubation with Camostat and EGTP; and

FIG. 5B-E illustrate confocal laser scanning microscopy (CLSM) images and (graphically, to the right of the images) the relative fluorescence intensities of TMPRSS2-Vero cells with different concentrations of Camostat and EGTP for cell imaging: (FIG. 5B) 0 mM Camostat, (FIG. 5C) 10 mM Camostat, (FIG. 5D) 100 mM Camostat, and (FIG. 5E) 1000 mM Camostat, and the EGTP is activated in the yellow channel when cells express TMPRSS2 without Camostat inhibition,
as discussed in further detail in Example 1, below.

FIG. 6A-B and FIG. 7 illustrate a protease-responsive and surface-potential-tunable peptide-conjugated AIEgen (EGTP) for TMPRSS2 selective imaging and accurate inhibitor screening in living cells is presented. Combining with mitifunctional peptides and AIEgens, the rational construction, catalytic efficiency, structural and surface-potential change, and intracellular distribution of EGTP are exploited with recombinant TMPRSS2, the MCF-7-GFP cells, TMPRSS2-Vero cells and TMPRSS2 inhibitors, as discussed in further detail in Example 1, below.

FIG. 8A illustrates exemplary synthetic Scheme S1, the synthetic route of PyTPE: the main synthetic route of PyTPE is three steps: (i) Synthesis of Py-I; (ii) Synthesis of Py-N3; (iii) Synthesis of PyTPE, High Resolution Mass Spectrometry (HRMS) (ESI) of PyTPE, as discussed in further detail in Example 2, below;

FIG. 8B illustrates exemplary synthetic Scheme S2, the main synthetic route of EGTP, which is two steps through standard solid phase Fmoc synthesis and a copper-catalyzed azide-alkyne click reaction: (i) Synthesis of EGT, electrospray ionization mass spectrometry (ESI-MS) (ESI+) (ii) Synthesis of EGTP, as discussed in further detail in Example 2, below;

FIG. 8C illustrates exemplary synthetic Scheme S3, the main synthetic route of TGP, which is two steps through standard solid phase Fmoc synthesis and a copper-catalyzed azide-alkyne click reaction: (i) Synthesis of TG, ESI-MS, and (ii) synthesis of TGP (i.e., the peptide QARGG (SEQ ID NO:2) linked by Propargylglycine (Pra) to the dye PyTPE), as discussed in further detail in Example 2, below; and

FIG. 8D illustrates exemplary synthetic Scheme S4, the main synthetic route of EGEP, which is two steps through standard solid phase Fmoc synthesis and a copper-catalyzed azide-alkyne click reaction: (i) Synthesis of EGE, ESI-MS (ESI−) m/z; (ii) synthesis of EGEP, ESI-MS (ESI−), as discussed in further detail in Example 2, below.

FIG. 9 graphically illustrates data from an HPLC of TGP, EGTP, and EGEP under the absorption wavelength of 405 nm, as discussed in further detail in Example 2, below.

FIG. 10A-C graphically illustrate the positive mode (FIG. 10A) and a negative mode electrospray ionization mass spectrometry (ESI-MS) (FIG. 10B), and high-resolution mass spectrometry (HR-MS) results (FIG. 10C) of PyTPE, as discussed in further detail in Example 2, below.

FIG. 11A-B illustrates the positive mode High Resolution Mass Spectrometry (HRMS) results of EGTP with two positive charges, see FIG. 11B (see FIG. 11A for structure EGTP), as discussed in further detail in Example 2, below.

FIG. 12A-B illustrate the negative mode ESI-MS results of TG (FIG. 12A) and TGP (FIG. 12B), as discussed in further detail in Example 2, below.

FIG. 13 illustrates the positive mode HRMS results of TGP with two positive charges, as discussed in further detail in Example 2, below.

FIG. 14 illustrates the negative mode ESI-MS results of compounds TGP after incubation with TMPRSS2 for 1 h showing that TGP can be cleaved by TMPRSS2, as discussed in further detail in Example 2, below.

FIG. 15A-B illustrate the negative mode ESI-MS results of EGE (FIG. 15A) and EGEP (FIG. 15B), as discussed in further detail in Example 2, below.

FIG. 16 illustrates the positive mode HRMS results of EGEP with two positive charges, as discussed in further detail in Example 2, below.

FIG. 17 illustrates the negative mode ESI-MS results of compounds EGEP after incubation with TMPRSS2 for 1 h showing that EGEP cannot be cleaved by TMPRSS2, as discussed in further detail in Example 2, below.

FIG. 18A-C illustrates the (FIG. 18A) The hydrodynamic size of (FIG. 18A) M PyTPE, (FIG. 18B) 20 μM TGP and 20 μM TGP with 100 nM TMPRSS2, and (FIG. 18C) 20 μM EGEP and (FIG. 18C) 20 μM EGEP with 100 nM TMPRSS2 in 20 mM Tris-HCl buffer, as discussed in further detail in Example 2, below.

FIG. 19A-D illustrate Zeta potential values of different concentrations of (FIG. 19A) PyTPE, (FIG. 19B) EGTP, (FIG. 19C) TGP, and (FIG. 19D) EGEP, suggesting good linear change of Zeta potential with increasing concentration of AIEgens, as discussed in further detail in Example 3, below.

FIG. 20A-F illustrate Zeta potential values of different concentrations of EGTP with 100 nM TMPRSS2, suggesting the Zeta potential became more positive after incubation with TMPRSS2:

FIG. 20A illustrates a schematic diagram of Zeta potential changes of EGTP with TMPRSS2; and

FIG. 20B-F illustrates Tris-HCl buffer (FIG. 20B), (FIG. 20C) 1 μM EGTP with/without TMPRSS2, (FIG. 20D) 5 μM EGTP with/without TMPRSS2, (FIG. 20E) 10 μM EGTP with/without TMPRSS2, (FIG. 20F) 20 μM EGTP with/without TMPRSS2,
as discussed in further detail in Example 3, below.

FIG. 21A-E illustrates transmission electron microscope (TEM) images of (FIG. 21A) 20 μM PyTPE, (FIG. 21B) 20 μM TGP, (FIG. 21C) 20 μM TGP with 100 nM TMPRSS2, (FIG. 21D) 20 μM EGEP, and (FIG. 21E) 20 μM EGEP with 100 nM TMPRSS2 in 20 mM Tris-HCl buffer, as discussed in further detail in Example 3, below.

FIG. 22A-E illustrate (FIG. 22A and FIG. 22C) Absorption, and (FIG. 22B, FIG. 22D, and FIG. 22E) fluorescence spectra of different concentration of PyTPE and EGTP showed the good solubility enhancement with the decreased fluorescence intensity of EGTP in 20 mM Tris-HCl buffer, as discussed in further detail in Example 3, below.

FIG. 23A-H graphically illustrate time-dependent fluorescence spectra of different concentration of EGTP (at concentrations as indicated in figures: FIG. 23A EGTP at 1 nM; FIG. 23B EGTP at 5 nM; FIG. 23C EGTP at 10 nM; FIG. 23D EGTP at 20 nM; FIG. 23E EGTP at 30 nM; FIG. 23F EGTP at 40 nM; FIG. 23G EGTP at 50 nM; and FIG. 23H summarizing the data from FIG. 23A-G) incubation with TMPRSS2 at 100 nM in 20 mM Tris-HCl buffer showing that the concentration of EGTP more than 20 μM with TMPRSS2 can display strong fluorescence in 20 mM Tris-HCl buffer, as discussed in further detail in Example 3, below.

FIG. 24 graphically illustrates Time-dependent fluorescence spectra of 20 μM EGTP incubation with different concentrations of TMPRSS2 in 20 mM Tris-HCl buffer showing that high concentrations of TMPRSS2 can produce strong fluorescence of the same concentration of EGTP, as discussed in further detail in Example 3, below.

FIG. 25 illustrates Time-dependent fluorescence spectra of 20 μM EGTP incubation with 20 mM Tris-HCl buffer, DMEM, and DMEM with 10% FBS showing that high concentrations of EGTP can aggregate easily with strong fluorescence in DMEM with 10% FBS, as discussed in further detail in Example 3, below.

FIG. 26A illustrates an exemplary experimental scheme; and

FIG. 26B-D illustrate confocal laser scanning microscopy (CLSM) images and graphically illustrate average fluorescence intensities (RFU, see the graphs to the right of the images) of (FIG. 26B) A549 cells, (FIG. 26C) HeLa cells, (FIG. 26D) MCF-7-GFP cells, and (FIG. 26E) MCF-7-GFP and A549 cells with ALEXA 647™ labeled TMPRSS2 antibodies and 5 μM HOECHST 33258™ for 0.5 hour,
as discussed in further detail in Example 3, below.

FIG. 27A-C illustrates metabolic activity of (FIG. 27A) MCF-7-GFP cells, (FIG. 27B) HeLa cells, and (FIG. 27C) A549 cells were studied with (1 μM, 5 μM, 10 μM, and 20 μM) PyTPE, (1 μM, 5 μM, 10 μM, and 20 μM) EGT, (1 μM, 5 μM, 10 μM, and 20 μM) EGTP, (1 μM, 5 μM, 10 μM, and 20 μM) TGP, (1 μM, 5 μM, 10 μM, and 20 μM) EGEP for 48 h incubation using a fluorescence resazurin assay, as discussed in further detail in Example 3, below.

FIG. 28A illustrates an exemplary experimental scheme; and

FIG. 28B-C illustrate CLSM images and average fluorescence intensities (RFU, see the graphs to the right of the images) of (FIG. 28B) MCF-7-GFP cells and (FIG. 28C) MCF-7-GFP and A549 cells with ALEXA 647™-labeled TMPRSS2 antibodies, 5 μM EGTP for 1 h, and 5 μM HOESCHT 33258™ for 0.5 h; in FIG. 28C the white and red dotted rings denote the MCF-7-GFP cells and A549 cells, respectively, as discussed in further detail in Example 3, below.

FIG. 29A-E illustrate CLSM images and average fluorescence intensities of A549 cells (FIG. 29A), HeLa cells (FIG. 29B), MCF-7-GFP cells (FIG. 29C), and MCF-7-GFP and A549 cells (FIG. 29D) with 10 μM TGP for 1 h, and 5 μM HOESCHT 33258™ for 0.5 h; and in FIG. 29D the white and red dotted rings denote the MCF-7-GFP cells and A549 cells, respectively; and

FIG. 29E graphically illustrates the data from FIG. 29A-E,
as discussed in further detail in Example 3, below.

FIG. 30A-B illustrate CLSM images and average fluorescence intensities of MCF-7-GFP cells, and MCF-7-GFP and A549 cells with 10 μM EGEP for 1 h, and 5 μM HOESCHT 33258™ for 0.5 h; in FIG. 30B the white and red dotted rings denote the MCF-7-GFP cells and A549 cells, respectively; and

FIG. 30C graphically illustrates the data from FIG. 30A-B,
as discussed in further detail in Example 3, below.

FIG. 31A-D illustrate CLSM images and average fluorescence intensities of Vero cells incubated with different concentration of PyTPE (FIG. 31A, 1 μM; FIG. 31B, 5 μM; FIG. 31C, 10 μM; FIG. 31D, 20 μM) for 1 h, then added HOESCHT 33258™ for 0.5 h, and

FIG. 31E graphically illustrates the average fluorescence intensities (RFU) seen in FIG. 31A-D,
as discussed in further detail in Example 4, below.

FIG. 32A-D illustrates CLSM images and average fluorescence intensities of Vero cells incubated with different concentration of EGTP (FIG. 32A, 1 μM; FIG. 32B, 5 μM; FIG. 32C, 10 μM; FIG. 32D, 20 μM) for 1 h, then added HOESCHT 33258™ for 0.5 h, and

FIG. 32E graphically illustrates the average fluorescence intensities (RFU) seen in FIG. 32A-D,
as discussed in further detail in Example 4, below.

FIG. 33A-D illustrate CLSM images and average fluorescence intensities of TMPRSS2-Vero cells incubated with different concentration of PyTPE (FIG. 33A, 1 μM; FIG. 33B, 5 μM; FIG. 33C, 10 μM; FIG. 33D, 20 μM) for 1 h, then added HOESCHT 33258™ for 0.5 h, and

FIG. 33E graphically illustrates the average fluorescence intensities (RFU) seen in FIG. 33A-D,
as discussed in further detail in Example 4, below.

FIG. 34A-D illustrate CLSM images and average fluorescence intensities of TMPRSS2-Vero cells incubated with different concentration of EGT PyTPE (FIG. 34A, 1 μM; FIG. 34B, 5 μM; FIG. 34C, 10 μM; FIG. 34D, 20 μM) P for 1 h, then added HOESCHT 33258™ for 0.5 h, and

FIG. 34E graphically illustrates the average fluorescence intensities (RFU) seen in FIG. 34A-D,
as discussed in further detail in Example 4, below.

FIG. 35A illustrates an exemplary experimental scheme; and

FIG. 35B-C illustrate CLSM images, and average fluorescence intensities (RFU, see the graphs to the right of the images) of TMPRSS2-Vero cells incubated with (FIG. 34B) 100 nM GC376 and (FIG. 34C) 100 nM GRL0617 for 1 h, 10 μM EGTP for 1 h, then added HOESCHT 33258™ for 0.5 h, as discussed in further detail in Example 4, below.

FIG. 36A-B illustrate the structure and function of the exemplary PSGMR: (FIG. 36A) Molecular structure of PSGMR; and (FIG. 36B) PSGMR is used for main protease (M^{pro}) detection and selective inhibition of M^{pro} plasmid transfected or SARS-CoV-2 infected cells, as discussed in further detail in Example 4, below.

FIG. 37A illustrates the peptide sequences (note: (Pra) is Propargylglycine):

| | |
|---|---|
| PMR | (Pra)GSAVLQ/SGFRKMAGRRRRRR |
| | (SEQ ID NO: 16) |
| PSMR | (Pra)KLVFFGSAVLQ/SGFRKMARRRRRR |
| | (SEQ ID NO: 17) |
| PSGMR | (Pra)KLVFFGGGSAVLQ/SGFRKMAGGGRRRRRR |
| | (SEQ ID NO: 18) |

FIG. 37B-I graphically illustrate characteristics of PSGMR and its derivatives: (FIG. 37B) high-performance liquid chromatography (HPLC); (FIG. 37C) absorption; (FIG. 37D) fluorescence spectra of PSGMR, PSMR and PMR showed the good purity and solubility enhancement with the decreased fluorescence intensity; (FIG. 37E) fluorescence spectra and (FIG. 37F) kinetics of PSGMR with M^{pro} and 10 mM M^{pro} GC376 at 590 nm showed the fluorescence increase because of M^{pro}; (FIG. 37G) Probe specificity of PSGMR with 200 nM different proteins including M^{pro}, papain-like protease (PL^{pro}), thrombin (TB), bovine serum albumin (BSA), and hemoglobin (HGB); (FIG. 37H) Hydrodynamic sizes; (FIG. 37I) zeta potential values; and

FIG. 37J illustrates transmission electron microscope (TEM) images of PSGMR (left image) and PSGMR with M^{pro} (right image),
as discussed in further detail in Example 3, below.

FIG. 38A illustrates an exemplary experimental scheme; of different plasmid transfected HEK 293T cells after incubation with PI, and PSGMR, where the enlarged portion (right image) shows the imaging mechanism of FLIPGFP™;

FIG. 38B-G illustrates data validating PSGMR via plasmids and reporter, and illustrate confocal laser scanning microscopy (CLSM) images of the plasmid transfected HEK 293T cells with (FIG. 38B, FIG. 38D, FIG. 38D) Mpro-related FLIPGFP™ reporter plasmid, (FIG. 38C) PR8 plasmid, (FIG. 38D, FIG. 38E, FIG. 38R, FIG. 38G) Mpro plasmid and PSGMR for different incubation time; and

FIG. 38H-J graphically illustrates average fluorescence intensities of GFP (FIG. 38H), PI (FIG. 38I) and PSGMR (FIG. 38J),
as discussed in further detail in Example 3, below.

FIG. 39A illustrates an exemplary experimental scheme of SARS-CoV-2 infected TMPRSS2-Vero cells incubation with PSGMR, anti-SARS-CoV-2 nucleocapsid (Capsid) primary antibody, anti-SARS-CoV-2 Mpro primary antibody, and ALEXAFLUOR 488™-labeled secondary antibody (ALEXA 488™), and the enlarged portion shows the imaging mechanism of ALEXA 488™; and

FIG. 39B-E illustrate selective imaging and inhibition of SARS-CoV-2 infected TMPRSS2-Vero cells with PSGMR and illustrate CLSM images and average fluorescence intensities (RFU, see the graphs to the right of the images) of:

FIG. 39B noninfected; and infected cells (FIG. 39C, FIG. 39D, FIG. 39E); incubation with (FIG. 39B, FIG. 39D, FIG. 39E) PSGMR, (FIG. 39B, FIG. 39C, FIG. 39D) capsid primary antibody/ALEXA 488™, and (FIG. 39E) Mpro primary antibody/ALEXA 488™,
as discussed in further detail in Example 3, below.

FIG. 40A illustrates an exemplary experimental scheme of SARS-CoV-2 infected TMPRSS2-Vero cells with PSGMR and Mpro inhibitor; and

FIG. 40B-F illustrate imaging and inhibition of SARS-CoV-2 infected TMPRSS2-Vero cells with PSGMR and Mpro inhibitor, and illustrate CLSM images of SARS-CoV-2 infected cells with 10 μM PSGMR (FIG. 40B) and 10 μM PMR (FIG. 40C); 10 μM PSGMR and 2 μM GC376 (FIG. 40D); 10 μM PSGMR and 6 μM GC376 (FIG. 40E); 10 μM PSGMR and 12 μM GC376 (FIG. 40F); in (FIG. 40B) and (FIG. 40C), the transverse and vertical dotted line denotes the XZ and YZ plane cutting line of Z-stack images, respectively, and the white boxes in the 2.5 D images are magnified, and red scale bar is 20 μm; and

FIG. 40G-H illustrate mean fluorescence intensity of PI (FIG. 40G) and PSGMR (FIG. 40H) in infected cells incubation with 10 μM PSGMR and different concentrations of GC376,
as discussed in further detail in Example 3, below.

FIG. 41 illustrates the mechanism of PSGMR was used for selective imaging and inhibition of Mpro plasmid-transfected or SARS-CoV-2-infected cells; the amphipathic PSGMR has 13 positive charges and can form loose nanoparticles due to the electrostatic repulsion; after cleavage by Mpro and the absence of cationic polypeptides, the resulting PSG has four positive charges and a C-terminal carboxyl group to decrease the electrostatic repulsion and enhance the molecular interactions; and the electrostatic attraction, decreased hydrophilicity, and increased self-assembly all increase aggregation of PSG to form nanofibers with strong yellow fluorescence.

FIG. 42 illustrates the synthetic route of PyTPE: the main synthetic route of PyTPE is three steps: (i) Synthesis of Py-I; (ii) Synthesis of Py-N3, (iii) Synthesis of PyTPE, as further described in Example 4, below.

FIG. 43A-C illustrate the synthetic route of PSMR in three steps through standard solid phase Fmoc synthesis and copper-catalyzed azide-alkyne click reaction: (FIG. 43A) Synthesis of SMM-P; (FIG. 43B) Synthesis of SMR, and (FIG. 43C) Synthesis of PSMR, as further described in Example 4, below.

FIG. 44A-C illustrate the synthetic route of PSGMR in three steps through standard solid phase Fmoc synthesis and copper-catalyzed azide-alkyne click reaction: (FIG. 44A) Synthesis of SGMR-P; (FIG. 44B) Synthesis of SGMR; and (FIG. 44C) Synthesis of PSGMR, as further described in Example 4, below.

FIG. 45A-C illustrate the synthetic route of PMR in three steps through standard solid phase Fmoc synthesis, thiol-maleimide Michael addition and copper-catalyzed azide-alkyne click reaction: (FIG. 45A) Synthesis of MR-P. (FIG. 45B) Synthesis of MR; and (FIG. 45C) Synthesis of PMR, as further described in Example 4, below.

FIG. 46A-D graphically illustrate HPLC results of different peptides before and after incubation with Mpro showing that some AVLQ (SEQ ID NO: 20) peptide sequence cannot be cleaved by Mpro; (FIG. 46A) CGAVLQDDD (SEQ ID NO:11) was not cleaved by Mpro; (FIG. 46B) AVLQFFVLKC (SEQ ID NO:12) was not cleaved by Mpro; (FIG. 46C) RVRRSAVLQSGFRKMAC (SEQ ID NO:13) was cleaved by Mpro; and (FIG. 46D) CGKLVFFGTSAVLQSGFRGDDD (SEQ ID NO: 14) was cleaved by Mpro, as further described in Example 4, below.

FIG. 47A-D graphically illustrate HPLC and ESI-MS results of compound RVRRSAVLQSGFRKMAC (SEQ ID NO:13) before and after incubation with M^{pro} showing that RVRRSAVLQSGFRKMAC (SEQ ID NO:13) can be cleaved by M^{pro} after AVLQ (SEQ ID NO: 20); (FIG. 47A) ESI-MS results of RVRRSAVLQ (SEQ ID NO: 21); (FIG. 47B) ESI-MS results of SGFRKMAC (SEQ ID NO: 22); (FIG. 47C) HPLC results of RVRRSAVLQSGFRKMAC (SEQ ID NO:13) before and after incubation with M^{pro}; (FIG. 47D) ESI-MS results of RVRRSAVLQSGFRKMAC (SEQ ID NO:13), as further described in Example 4, below.

FIG. 48A-C illustrates HPLC and ESI-MS results of compound CGKLVFFGTSAVLQSGFRGDDD (SEQ ID NO:14) before and after incubation with M^{pro} showing that CGKLVFFGTSAVLQSGFRGDDD (SEQ ID NO: 14) can be cleaved by M^{pro} after AVLQ (SEQ ID NO: 20): (FIG. 48A) HPLC results of CGKLVFFGTSAVLQSGFRGDDD (SEQ ID NO:14) before and after incubation with M^{pro} (FIG. 48B) ESI-MS results of CGKLVFFGTSAVLQ (SEQ ID NO:8); and, (FIG. 48C) ESI-MS results of SGFRGDDD (SEQ ID NO:15), as further described in Example 4, below.

FIG. 49A-F graphically illustrate HPLC results of compounds PSGMR, PMR, and PSMR before and after incubation with M^{pro} showing that PSGMR and PMR not PSMR can be cleaved by M^{pro}; (FIG. 49A and FIG. 49B) PSGMR; (FIG. 49C and FIG. 49D) PMR; (FIG. 49E and FIG. 49F) PSMR, as further described in Example 4, below.

FIG. 50A-I illustrate ESI-MS results of compounds PSGMR, PSMR, and PMR before and after incubation with M^{pro} for 1 h and 24 h showing that PSGMR and PMR but not PSMR can be cleaved by M^{pro}. (FIG. 50A, FIG. 50D, FIG. 50G) PSGMR; (FIG. 50B, FIG. 50E, FIG. 50H) PSMR; (FIG. 50D, FIG. 50F, FIG. 50I) PMR, as further described in Example 4, below.

FIG. 51A-D graphically illustrate time-dependent fluorescence spectra of (FIG. 51A, FIG. 51C) 5 μM and (FIG. 51B, FIG. 51D) 10 μM PSGMR incubation with M^{pro} in 20 mM Tris-HCl buffer and DMEM showing that high concentrations of PSGMR can aggregate easily with strong fluorescence both in 20 mM Tris-HCl buffer and DMEM, as further described in Example 4, below.

FIG. 52A-D graphically illustrate time-dependent fluorescence spectra of (FIG. 52A, FIG. 52C) 5 μM and (FIG. 52B, FIG. 52D) 10 μM PMR incubation with M^{pro} in 20 mM Tris-HCl buffer and DMEM showing that high concentrations of PMR can easily aggregate with strong fluorescence both in 20 mM Tris-HCl buffer and DMEM, as further described in Example 4, below.

FIG. 53 graphically illustrates time-dependent fluorescence spectra of 10 μM PSMR incubation with M^{pro} in 20 mM Tris-HCl buffer showing that PSMR cannot aggregate with weak fluorescence in 20 mM Tris-HCl buffer, as further described in Example 4, below.

FIG. 54A-B illustrate (FIG. 54A) transmission electron microscope (TEM) images and (FIG. 54B) graphically illustrate hydrodynamic size of 20 mM Tris-HCl buffer, as further described in Example 4, below.

FIG. 55A-B illustrate (FIG. 55A) Transmission electron microscope (TEM) images and (FIG. 55B) graphically illustrate hydrodynamic sizes of 1 μM PSGMR incubated with and without M^{pro} in 20 mM Tris-HCl buffer, where the enlarged section (inset) illustrates a clearer image of nanostructures and their aggregation; as further described in Example 4, below.

FIG. 56A-B illustrate (FIG. 56A) Transmission electron microscope (TEM) images and (FIG. 56B) graphically illustrate hydrodynamic sizes of 10 μM PSGMR incubated with and without M^{pro} in 20 mM Tris-HCl buffer, where the enlarged section (inset) illustrates a clearer image of nanostructures and their aggregation, as further described in Example 4, below.

FIG. 57A-B illustrate (FIG. 57A) Transmission electron microscope (TEM) images and (FIG. 57B) graphically illustrate hydrodynamic sizes of 100 μM PSGMR incubated with and without M^{pro} in 20 mM Tris-HCl buffer, where the enlarged section (inset) illustrates a clearer image of nanostructures and their aggregation, as further described in Example 4, below.

FIG. 58A-B illustrate (FIG. 58A) Transmission electron microscope (TEM) images and (FIG. 58B) hydrodynamic sizes of 200 μM PSGMR incubated with and without M^{pro} in 20 mM Tris-HCl buffer, where the enlarged section (inset) illustrates a clearer image of nanostructures and their aggregation, as further described in Example 4, below.

FIG. 59A-B illustrate metabolic activity of HEK 293T cells and plasmid transfected HEK 293T cells, which were studied with (FIG. 59A) (1 μM, 5 μM, 10 μM, 20 μM, and 40 μM) PyTPE, (1 μM, 5 μM, 10 μM, 20 μM, and 40 μM) SGMR, (1 μM, 5 μM, 10 μM, 20 μM, and 40 μM) PSGMR; (FIG. 59B) (1 μM, 5 μM, 10 μM, and 20 μM) PMR (μM^{pro} plasmid), (1 μM, 5 μM, 10 μM, and 20 μM) PSGMR (μM^{pro} plasmid), and (1 μM, 5 μM, 10 μM, and 20 μM) PSGMR (PR8 plasmid) for 48 h incubation using a fluorescence resazurin assay, as further described in Example 4, below.

FIG. 60A illustrates an exemplary experimental scheme;

FIG. 60B-F illustrate Confocal laser scanning microscopy (CLSM) images of HEK 293T cells incubated with HOECHST 33258™, PSGMR, and PI: Cell images of HEK 293T cells incubated with (FIG. 60B) 1 μM, (FIG. 60C) 5 μM, (FIG. 60D) 10 μM, (FIG. 60E) 20 μM, and (FIG. 60F) 40 μM PSGMR, with 5 μM HOECHST 33258™, and 5 μM PI for 3 h, respectively; and

FIG. 60G graphically illustrates average fluorescence intensities of HEK 293T cells incubation with HOECHST 33258™, PSGMR, and PI., as further described in Example 4, below.

FIG. 61A-C illustrates CLSM images and average fluorescence intensities of M^{pro}-related FLIPGFP™ reporter plasmid, and M^{pro} plasmid transfected HEK 293T cells incubated with PSGMR, and PI, showing that the red, yellow and green fluorescence turn-on in these cells: FIG. 61A illustrates the exemplary experimental scheme; FIG. 60B illustrates CLSM images; and FIG. 60C graphically illustrates average fluorescence intensities of PI, PSGMR and GFP in each panel, as further described in Example 4, below.

FIG. 62A-E illustrate CLSM images and average fluorescence intensities of SARS-CoV-2 infected TMPRSS2-Vero cells incubation with HOECHST 33258™, PI, and PSGMR: FIG. 62A illustrates an exemplary experimental scheme, and cell images and average fluorescence intensities (in RFUs, see graphs to the right of the images) of (FIG. 63B, FIG. 63D) of (FIG. 62B, FIG. 62D) noninfected TMPRSS2-Vero cells incubation with 5 μM and 10 μM PSGMR, and (FIG. 62C, FIG. 62E) SARS-CoV-2 infected TMPRSS2-Vero cells incubation with 5 μM and 10 μM PSGMR, as further described in Example 4, below.

FIG. 63A-E illustrate CLSM images and average fluorescence intensities of SARS-CoV-2 infected TMPRSS2-Vero cells incubation with HOECHST 33258™, PI, and PMR: (FIG. 63A) illustrates an exemplary experimental scheme; and cell images and average fluorescence intensities (in RFUs, see graphs to the right of the images) of (FIG. 63B, FIG. 63D) noninfected TMPRSS2-Vero cells incubation with 5 μM and 10 μM PMR, and (FIG. 63C, FIG. 63E) SARS-CoV-2 infected TMPRSS2-Vero cells incubation with 5 μM and 10 μM PMR, as further described in Example 4, below.

FIG. 64A-C illustrate CLSM images and average fluorescence intensities of SARS-CoV-2 infected TMPRSS2-Vero cells incubation with HOECHST 33258™ PI, PSGMR, anti-SARS-CoV-2 nucleocapsid (Capsid) primary antibody, anti-SARS-CoV-2 M^{pro} primary antibody, and ALEXAFLUOR 488™-labeled secondary antibody (ALEXA 488™): FIG. 64A illustrates an exemplary experimental scheme; and cell images and average fluorescence intensities (in RFUs, see graphs to the right of the images) of SARS-CoV-2 infected TMPRSS2-Vero cells incubated with 5 μM PSGMR are illustrated (FIG. 64B) anti-SARS-CoV-2 Capsid primary antibody/ALEXA 488™, and (FIG. 64C) anti-SARS-CoV-2 M^{pro} primary antibody/ALEXA 488™, as further described in Example 4, below.

FIG. 65A-E illustrate ARS-CoV-2 infected TMPRSS2-Vero cells incubation with HOECHST 33258™, PI, PSGMR, and PMR:

FIG. 65A illustrates an exemplary experimental scheme;

FIG. 65B illustrates noninfected cells incubated with PSGMR; FIG. 65C illustreates SARS-CoV-2 infected cells incubation with PSGMR;

FIG. 65D illustrates SARS-CoV-2 infected cells incubation with PMR; and

FIG. 65E graphically illustrates the average fluorescence intensities of TMPRSS2-Vero cells incubated with Hoechst 33258, PI, PSGMR, and PMR, as further described in Example 4, below.

FIG. 66A-B illustrate images corresponding to the three-dimensional map of SARS-CoV-2 infected TMPRSS2-Vero cells incubated with 5 μM of (FIG. 66A) PSGMR and (FIG. 66B) PMR for 1 h, as further described in Example 4, below.

Like reference symbols in the various drawings indicate like elements.

### DETAILED DESCRIPTION

In alternative embodiments, provided is a protease-responsive and surface-potential-tunable peptide-conjugated AIEgens (EGTP) for TMPRSS2 selective imaging and accurate inhibitor screening, where EGTP comprises four segments: the first is a polyglutamic acid (Glu, E for short in EGTP) that increases the solubility, blocks the positive charges and cell-penetrating ability of PyTPE; the second comprises a spacer trimylglycine (GGG, G) designed to enhance probe flexibility and reduce steric hindrance for TMPRSS2-substrate interactions; the third component second comprises a TMPRSS2-responsive peptide (QAR, T), which can be cleaved by TMPRSS2 after QAR sequence; and the fourth second comprises a positive charged AIEgens (PyTPE, P).

Example 1, below, describes development of a TMPRSS2-responsive surface-potential-tunable peptide-conjugated probe (EGTP) with aggregation-induced emission (AIE) characteristic for TMPRSS2 selective imaging and accurate inhibitor screening. The amphiphilic EGTP was constructed with tunable surface potential and responsive efficiency with TMPRSS2 and its inhibitor. By rational construction of AIE luminogen (AIEgen) with modular peptides, we verified that the cleavage of EGTP yielded gradual aggregation with bright fluorescence in TMPRSS2 high-expression cells. This strategy has value for the selective detection of cancer cells and the SARS-CoV-2-host cells as well as accurate inhibitor screening.

In alternative embodiments, provided are main protease (Mpro)-responsive and modular-peptide-conjugated probes for the selective imaging and inhibition of SARS-CoV-2 infected cells via enzyme-instructed self-assembly and aggregation-induced emission. We exploited the potential advantages of EISA and the AIE effect for selective detection and treatment of the virus infected cells. When combined with SARS-CoV-2 replication characteristics, a Mpro-responsive modular peptide with conjugated AIEgens named “PSGMR” offers selective imaging and inhibition of the Mpro plasmid transfected HEK 293T cells and SARS-CoV-2 infected TMPRSS2-Vero cells.

In alternative embodiments, PSGMR (the Mpro-responsive modular (self-assembling) peptide with conjugated AIEgens) five segments comprises:
first is an AIEgen (PyTPE, P for short in PSGMR). PyTPE has bright yellow fluorescence, excellent biocompatibility, and good photostability;
second, the self-assembling peptide (KLVFF (SEQ ID NO:5), S) is a B-sheet-forming peptide derived from A-amyloid protein. It can spontaneously self-assemble into amyloid fibrils through π-π stacking, hydrogen bonding, and hydrophobic interactions;
third, the spacer trimylglycine (GGG, G) can enhance flexibility and reduce steric hindrance for Mpro-substrate interactions;
fourth component is the Mpro-responsive peptide (SAVLQ/SGFRKMA (SEQ ID NO:6), M), and
the fifth is a positive hexamolyarginine (RRRRRR (SEQ ID NO:7), R) that increases both the solubility and cell-penetrating ability of PSGMR.

These five components were covalently coupled through a Fmoc-based solid-phase peptide synthesis and a copper-catalyzed azide-alkyne click reaction. In the absence of Mpro, PSGMR is an amphiphilic molecule that is highly water-soluble with limited fluorescence. It can form loose nanoparticles due to the positive hexamolyarginine residues on the surface and hydrophobic core of PyTPE. After being cleaved by Mpro, however, the hydrophilic hexamolyarginine is separated from PSG, and the self-assembling peptides with one negative charge were exposed to the nanoparticle surface. The increasing self-assembly and electrostatic attraction as well as the decreasing hydrophilicity led to PSG aggregation and nanofibers with strong yellow fluorescence. Finally, the nanofibers can selectively inhibit the growth of SARS-CoV-2-infected cells and prevent virus replication.

As described in Example 3, severe acute respiratory syndrome coronavirus 2 (SARS-CoV-2) is a serious threat to human health without effective treat-ment. There is an urgent need for both real-time tracking and precise treatment of the SARS-CoV-2 infected cells to mitigate and ultimately prevent viral transmission. However, selective and responsive triggering and tracking of the therapeutic pro-cess in infected cells remains challenging. Here, we reported a series of main protease (Mpro)-responsive and modular-peptide-conjugated probes for the selective imaging and inhibition of SARS-CoV-2 infected cells via enzyme-instructed self-assembly (EISA) and aggregation-induced emission (AIE). The amphiphilic probe PSGMR was constructed with tunable structure and function and was validated with recombinant proteins, cells transfected with Mpro plasmid, and cells infected by SARS-CoV-2 in the presence and absence of Mpro inhibitors. By combining AIE luminogen (AIEgen) with modular peptides and Mpro, we verified, for the first time, that the cleavage of PSGMR by Mpro yielded nanofibers with bright fluorescence and enhanced cytotoxicity to the infected cells. This strategy provides for the selective detection and treatment of infected cells.

### Products of Manufacture and Kits

Provided are products of manufacture and kits comprising synthetic peptides as provided herein, for practicing methods as provided herein; and optionally, products of manufacture and kits can further comprise instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Protease-Responsive Surface-Potential-Tunable Peptide-Conjugated AIEgens for TMPRSS2 Selective Imaging and Accurate Inhibitor Screening

This example describes making and using protease-responsive and surface-potential-tunable peptide-conjugated AIEgens (EGTP) for TMPRSS2 selective imaging and accurate inhibitor screening as provided herein.

Transmembrane protease serine 2 (TMPRSS2) is an extracellular protease to activate both the spike protein of coronaviruses for cell entry and oncogenic signaling pathways for tumor progression. TMPRSS2 inhibition not only can effectively reduce cancer invasion and metastasis as well as partially prevent the entry of SARS-CoV-2 into host cells. There is an urgent need for both real-time tracking of TMPRSS2 expression and precise screening of TMPRSS2 inhibitors to cure cancer and ultimately prevent viral transmission. Here, we reported a TMPRSS2-responsive surface-potential-tunable peptide-conjugated probe (EGTP) with aggregation-induced emission (AIE) characteristic for TMPRSS2 selective imaging and accurate inhibitor screening. The amphiphilic EGTP was constructed with tunable surface potential and responsive efficiency with TMPRSS2 and its inhibitor. By rational construction of AIE luminogen (AIEgen) with modular peptides, we verified that the cleavage of EGTP yielded gradual aggregation with bright fluorescence in TMPRSS2 high-expression cells. This strategy provides for the selective detection of cancer cells and the SARS-CoV-2-host cells as well as accurate inhibitor screening.

To reduce non-specific aggregation of positive charged AIEgens inside cells, and make sure cleavage of probe by extracellular proteases before entering cells, the biomolecule-conjugated AIEgens could achieve these proteases selective imaging of targeted cells with protease-responsive peptides, polyanionic peptides, and positive charged AIEgens.

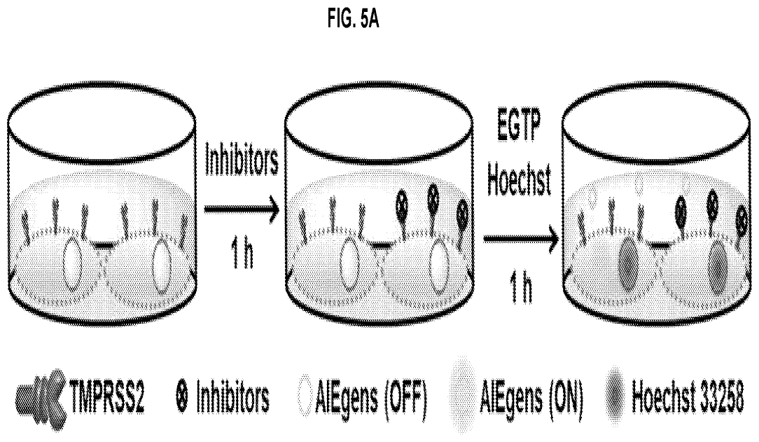

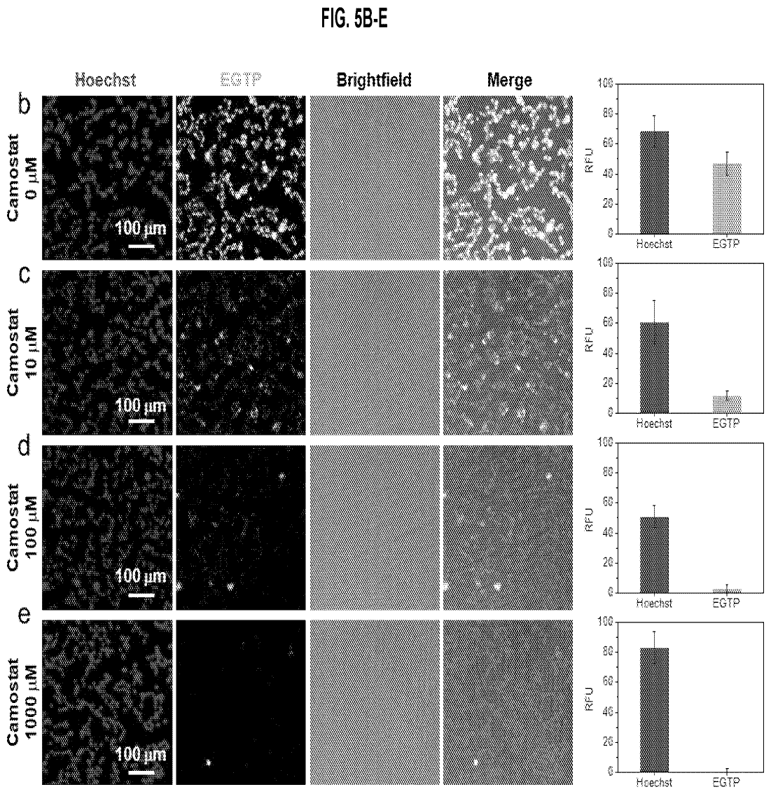

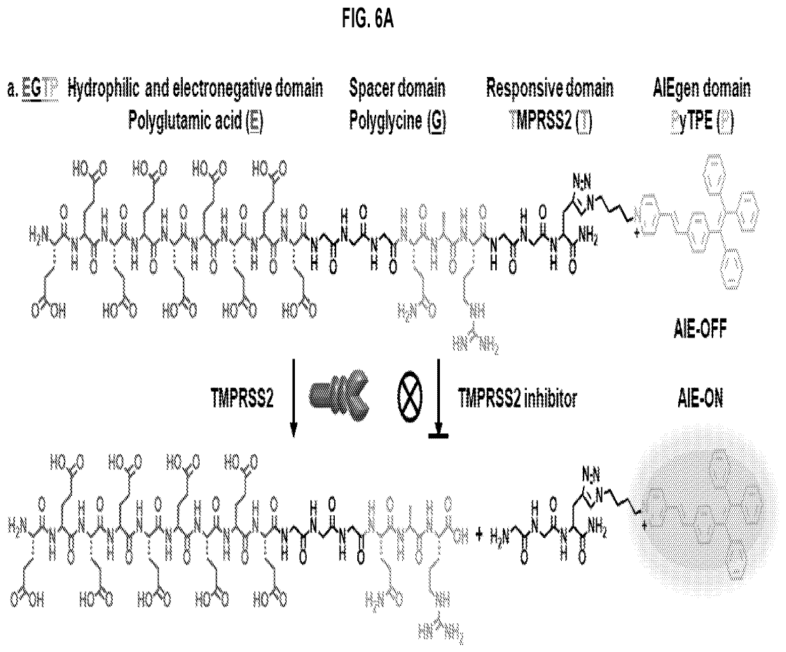

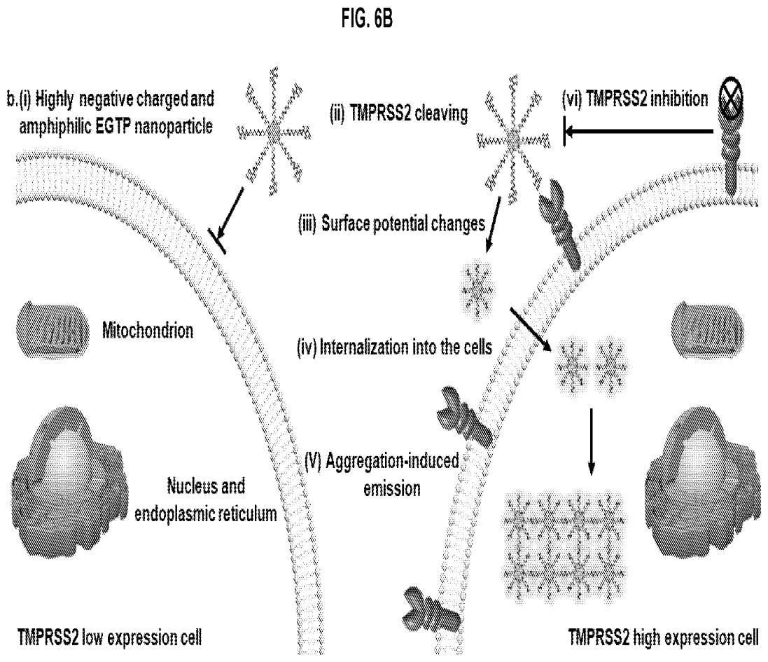

Herein, we developed a protease-responsive and surface-potential-tunable peptide-conjugated AIEgens (EGTP) for TMPRSS2 selective imaging and accurate inhibitor screening (Scheme 1, as illustrated in FIG. 6). EGTP consists of four segments: The first is a polyglutamic acid (Glu, E for short in EGTP) that increases the solubility, blocks the positive charges and cell-penetrating ability of PyTPE.⁵⁸⁻⁵⁹ Second, the spacer trimylglycine (GGG, G) is designed to enhance probe flexibility and reduce steric hindrance for TMPRSS2-substrate interactions.⁶⁰⁻⁶¹ The third component is the TMPRSS2-responsive peptide (QAR, T), which can be cleaved by TMPRSS2 after QAR sequence.^{10-11, 29} The fourth is a positive charged AIEgens (PyTPE, P).^{47, 53-55} These four components were covalently coupled through Fmoc-based solid-phase peptide synthesis and copper-catalyzed azide-alkyne click reaction. In the absence of TMPRSS2, EGTP as a highly negative charged and amphiphilic molecule can form loose nanoparticles with limited fluorescence (Scheme 1a). After being cleaved by TMPRSS2, the hydrophilic polyglutamic acid is separated from PyTPE part. The decreasing hydrophilicity led to the PyTPE part gradual aggregation with strong fluorescence. Once TMPRSS2 is suppressed by its inhibitors, no fluorescence could be observed. Thus, EGTP can only be cleaved to release the PyTPE part, across the cell membrane, and aggregate with enhanced fluorescence in the TMPRSS2 high expression cells rather than the TMPRSS2 low expression cells or incubation together with its inhibitor (Scheme 1b). This theranostic probe will provide a controllable avenue for TMPRSS2 selective imaging and accurate inhibitor screening in the cells.

FIG. 6, Scheme 1. Structure and function of EGTP. (a) Molecular structure changes of EGTP with TMPRSS2 and its inhibitor. (b) EGTP is used for TMPRSS2-responsive imaging and accurate inhibitor screening. i) The highly negative charged amphiphilic EGTP cannot cross the cell membrane of the TMPRSS2 low expression cells; ii) EGTP can be cleaved in the TMPRSS2 high expression cells; iii) After removal of the negative charged polyglutamic acid, the surface potential of PyTPE part changes; iv) The positive charged PyTPE part can internalizate into the cells; v) Large numbers of PyTPE part can aggregate with enhanced fluorescence; vi) Once TMPRSS2 is suppressed by its inhibitors, no fluorescence could be observed.

### Results and Discussion

### Design, Synthesis, and Characterization of EGTP and its Derivatives.

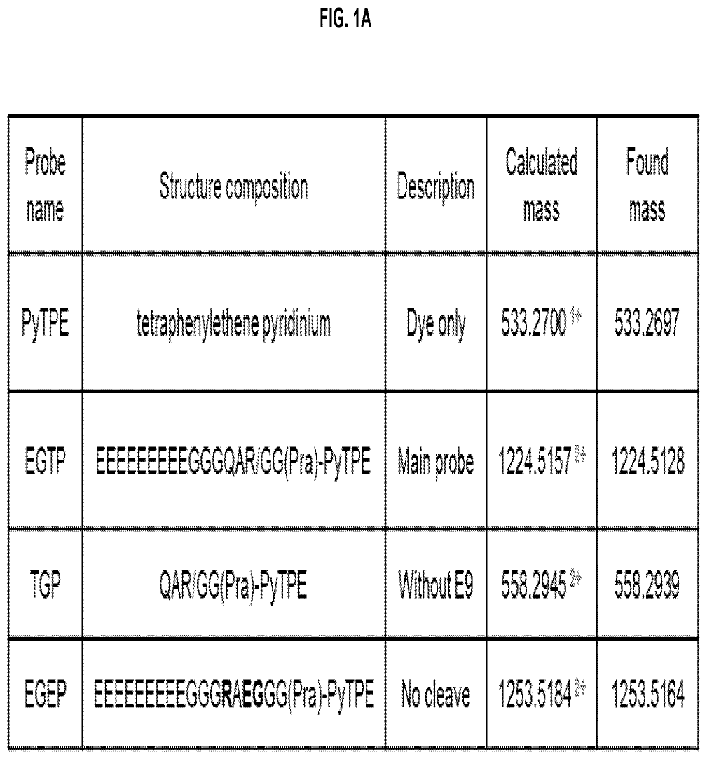

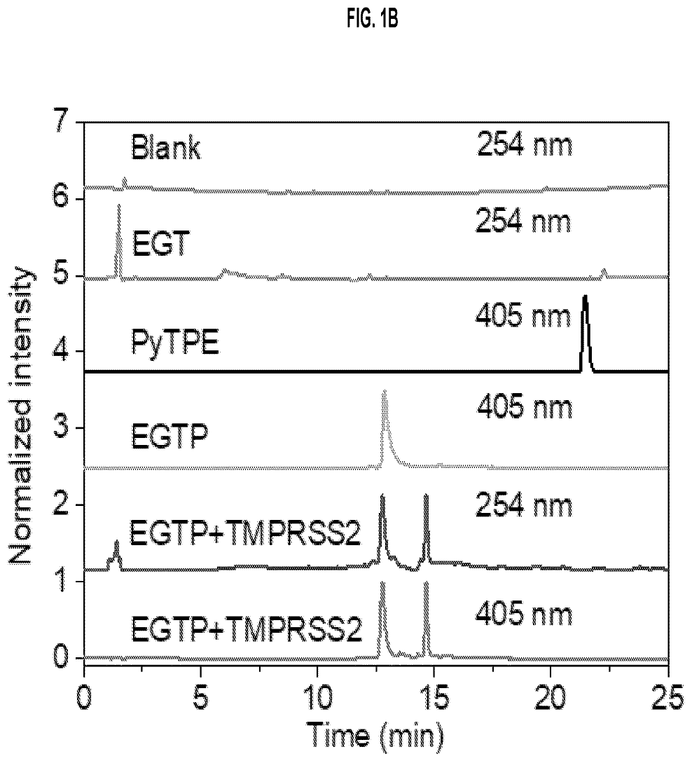

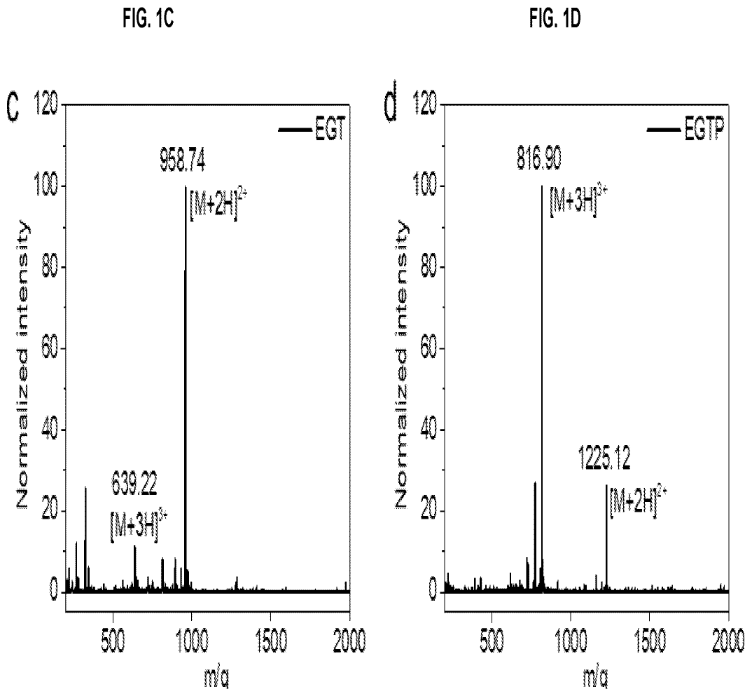

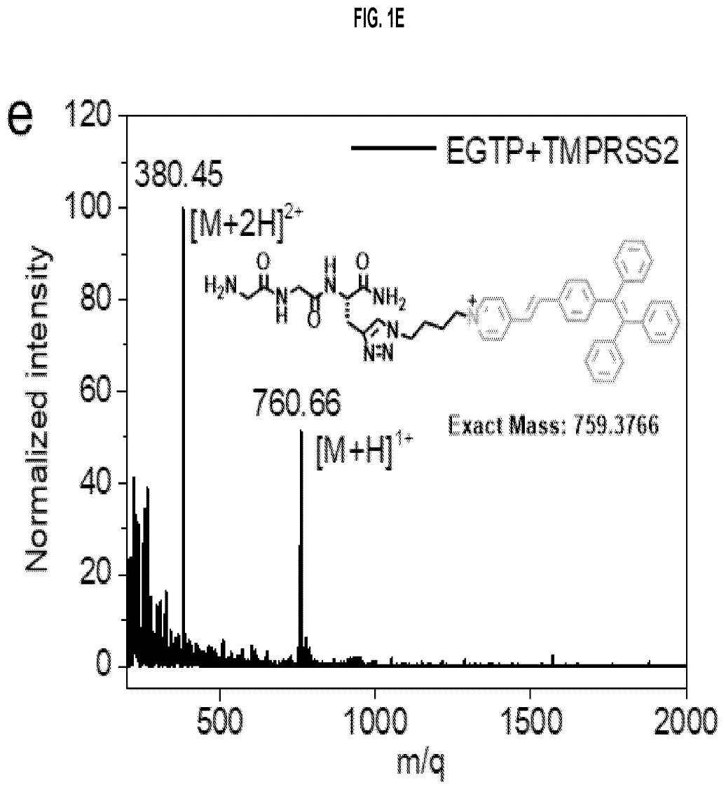

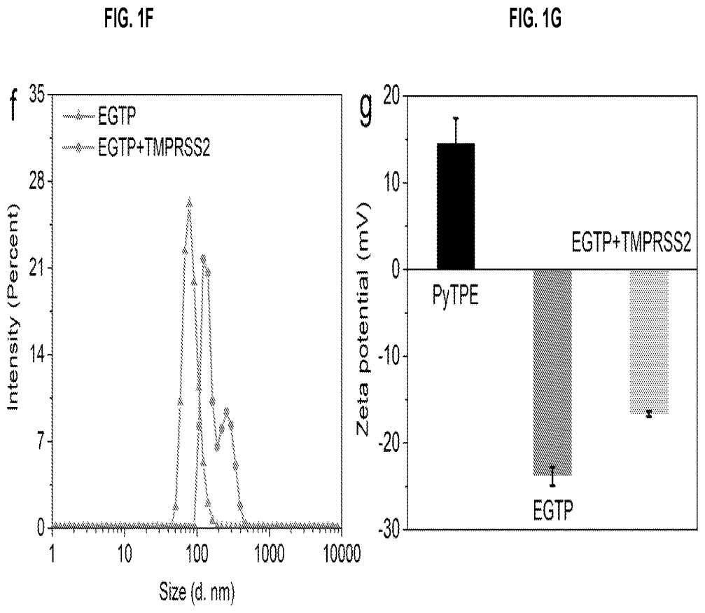

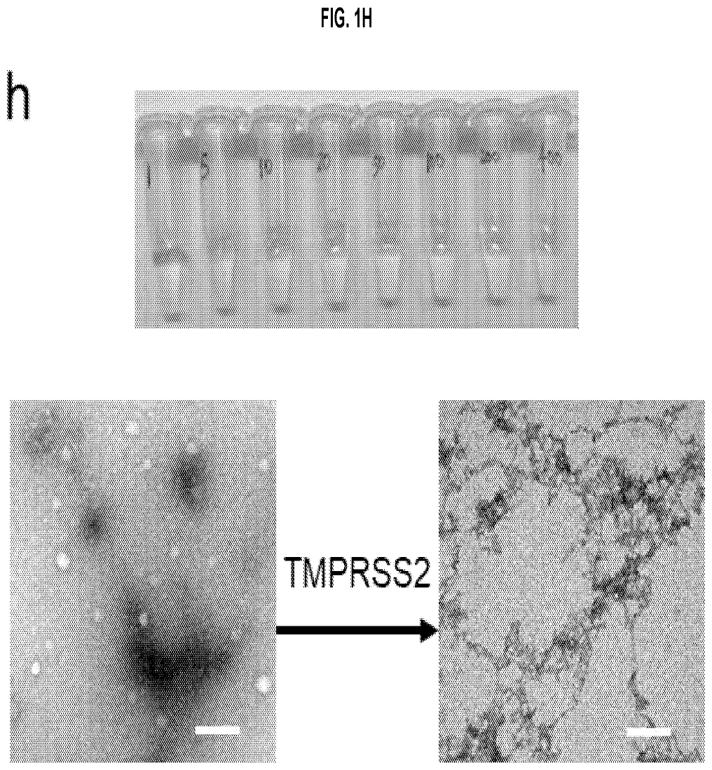

Surface-potential-tunable peptide-conjugated AIEgens (EGTP) were designed based on aggregation-induced emission (AIE) effect and activatable cell-penetrating via an TMPRSS2 trigger (FIG. 1a). The polyglutamic acid was placed at the N-terminal. Importanly, QAR sequence as a substrate for TMPRSS2 cleavage can change the ratio of hydrophobicity and hydrophilicity of EGTP in the middle. Trimylglycine was added at both ends of the TMPRSS2 substrate to leave space for enzyme and substrate binding. Propargylglycine (Pra) was used as a linker at the N-terminal to react with PyTPE (TetraPhenylEthene Pyridinium). All the domains were coupled via Fmoc-based solid-phase peptide synthesis and a copper-catalyzed click reaction. EGTP After TMPRSS2 cleavage, EGTP was divided into the hydrophobic GG(Pra)-PyTPE and the hydrophilic EEEEEEEEEGGGQAR (SEQ ID NO:4). Two control probes without polyglutamic acid (TGP) and TMPRSS2-responsive peptide (EGEP) were synthesized to verify the TMPRSS2 accessibility. All the probes were synthesized according to previous reports (Schemes S1-S4), characterized by high performance liquid chromatography (HPLC, FIG. 1b and S1), electrospray ionization mass spectrometry (ESI-MS, FIGS. 1c, 1d, 1e, S2, S4, and S7), and high-resolution mass spectra (HRMS, Figures S3, S5, and S8) to confirm their purity (at least 95%) and chemical structures. Taking EGTP for example, FIG. 1d showed a strong peak at 1225.12 attributed to the [M+2H]²⁺ ion of EGTP (calculated, 1224.51286); a strong peak at 816.90 attributed to the [M+3H]³⁺ ion of EGTP (calculated, 816.6815). The mass data of TGP and EGEP also matched well with the calculated data. These data indicated that EGTP, TGP, and EGEP were synthesized successfully.

FIG. 1. Structural characterization of EGTP and its derivatives. (a) Brief description of PyTPE, EGTP, TGP, and EGEP. (b) High-performance liquid chromatography (HPLC) results of EGT, PyTPE, EGTP, and EGTP incubation with TMPRSS2 under the 254 nm or 405 nm. (c-e) Electrospray ionization mass spectrometry (ESI-MS) results of EGT, EGTP, and EGTP incubation with TMPRSS2. (f) Hydrodynamic sizes, (g) zeta potential values, (h) photographs and transmission electron microscope (TEM) images of PyTPE and EGTP without and with TMPRSS2. In panel a, the red slash represents the TMPRSS2 cleavage position, the red superscript represents the number of charge in the probes. In panel h, vials contained 1, 5, 10, 20, 50, 100, 200, and 400 mM EGTP solutions. 20 μM of PyTPE, EGTP, TGP, and EGEP were dissolved in Tris-HCl buffer with 1% DMSO.

Responsiveness to enzyme in solutions. We first evaluated whether the probes can be specifically cleaved by recombinant TMPRSS2 as predicted. HPLC and ESI-MS analysis also confirmed that EGTP and TGP but not EGEP can be cleaved by TMPRSS2 between R and G after incubation for 1 h at 37° C. in 20 mM Tris-HCl buffer (pH 8.0) (FIGS. 1b, 1e, S6 and S9). Dynamic light scattering (DLS) tests and transmission electron microscopy (TEM) of EGTP, TGP, and EGEP were performed to determine the change of particle size, surface potential distribution, and particle morphology after incubation with TMPRSS2 (Figures S10 and S11). For 20 mM EGTP, the average hydrodynamic size increased from 79 nm to 352 nm (FIG. 1f), and the mean zeta potential value increased from −23.8 mV to −16.6 mV (FIG. 1g and S12), suggesting nanoparticle aggregation and a reduction of polyglutamic acid on the particle surface. Their morphology changes with TMPRSS2 incubation was confirmed by TEM (FIG. 1h and S13). These data proved that EGTP and TGP but not EGEP is responsive to TMPRSS2 leading to aggregation state and surface potential changes.

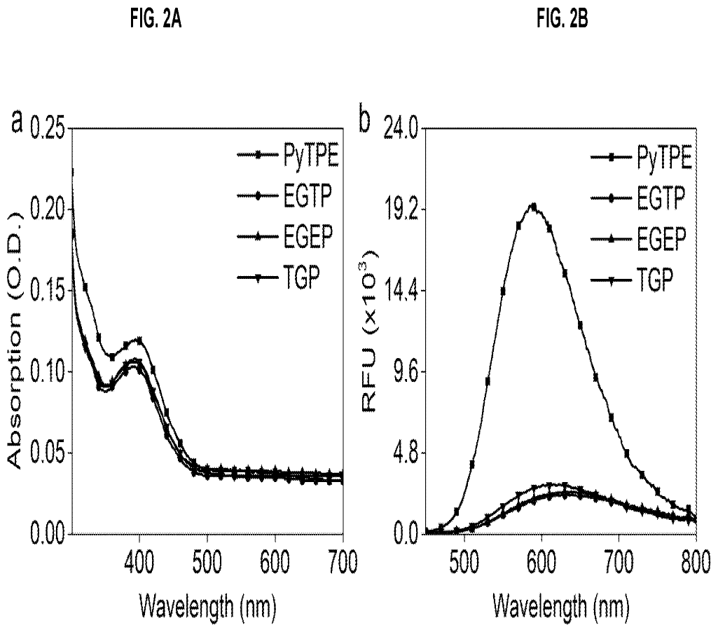

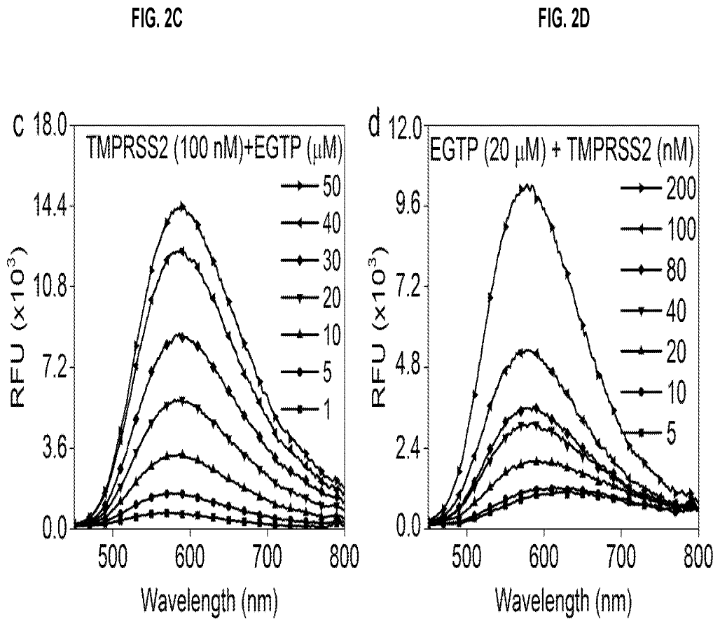

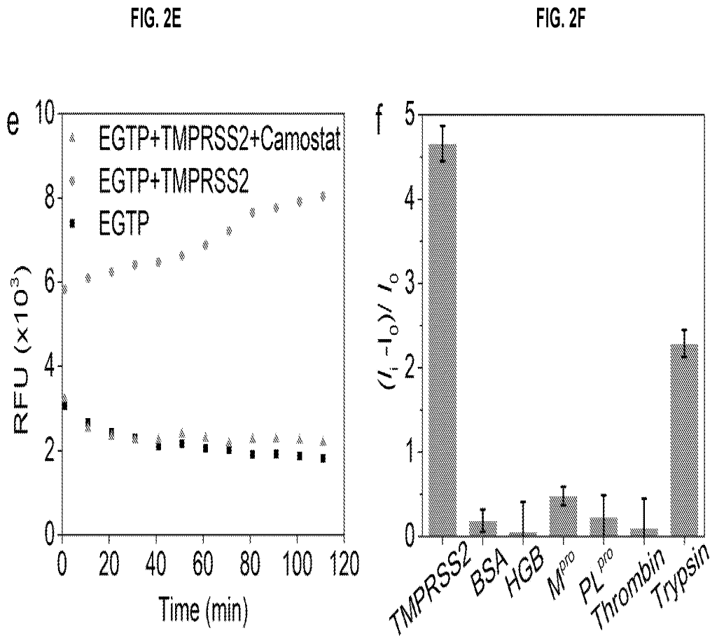

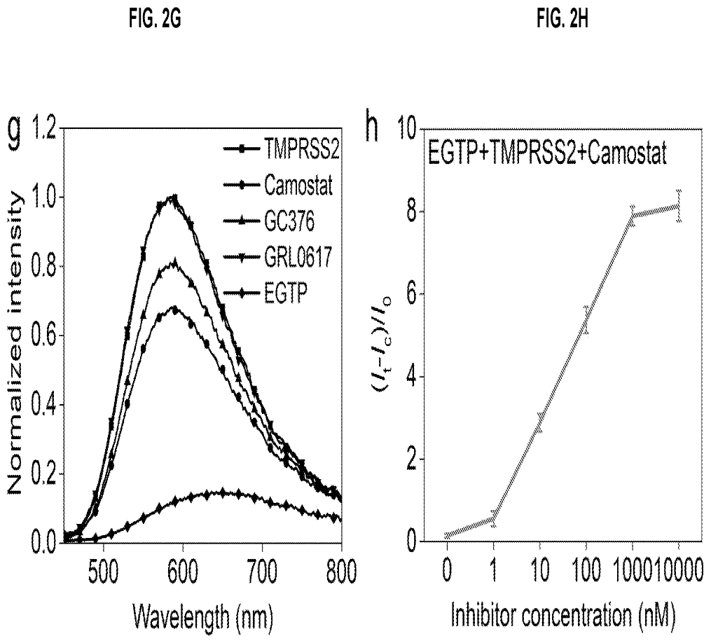

FIG. 2. Photophysical properties of EGTP and its derivatives. (a) absorption, and (b) fluorescence spectra of EGTP, TGP and EGEP showed the solubility enhancement with the decreased fluorescence intensity. (c, d) Fluorescence spectra and (e) kinetics of EGTP with different concentration of TMPRSS2 and 100 nM camostat (a serine protease inhibitor) at 590 nm showed the fluorescence increase because of TMPRSS2. (f) Probe specificity of EGTP with 200 nM different proteins including TMPRSS2, bovine serum albumin (BSA), hemoglobin (HGB), main protease (M^{pro}), papain-like protease (PL^{pro}), thrombin (TB), and trypsin. (g, h) Impact of protease inhibitors as studied with EGTP, TMPRSS2, camostat, GC376, and GRL0617. In panel h, I₀ is the fluorescence intensity of EGTP. It is the fluorescence intensity of EGTP after incubation with TMPRSS2. I_{c} is the fluorescence intensity of EGTP after incubation TMPRSS2 and different concentrations of camostat. 20 μM of PyTPE, EGTP, TGP, and EGEP were dissolved in Tris-HCl buffer with 1% DMSO.

We then explored the spectral properties of PyTPE, EGTP, TGP, and EGEP. They showed absorption spectral profiles at 320-490 nm in the Tris-HCl buffer with 1% DMSO (FIG. 2a, and S14); 405 nm was chosen as optimal excitation wavelength. The fluorescence spectral profiles of PyTPE, EGTP, TGP, and EGEP were 500-750 nm, and decreased after being modified with hydrophilic peptides. (FIG. 2b). Particularly, the fluorescence intensity of TGP after incubation with TMPRSS2 was stronger than that of EGTP and EGEP because the lack of polyglutamic acid. The fluorescence changes of EGTP were monitored upon incubation with TMPRSS2: 20 μM offered the significant fluorescence enhancement and was used for subsequent experiments (Figure S15). To validate the enzyme digestion efficiency, different concentration of EGTP was incubated with different concentrations of TMPRSS2 (FIGS. 2c, 2d, and S16). The fluorescence intensity of EGTP enhanced with increasing EGTP and TMPRSS2 concentration. While the fluorescence intensity of EGTP enhanced not as much as PyTPE at the same concentration. This is because of some hydrophilic residues (G and Pra) still linked with PyTPE after cleavage. Subsequent kinetic studies were performed by incubating EGTP with 100 nM TMPRSS2 and 100 nM camostat mesylate (camostat) over time (FIG. 2e).^{23, 32, 62} The fluorescence intensity of EGTP at 590 nm obviously increased with TMPRSS2 incubation for 120 min, and decreased without TMPRSS2 and with Camostat due to the photobleaching. EGTP was treated under identical conditions with several commercial proteins and different mediums to investigate probe specificity and stability: papain-like protease, thrombin, bovine serum albumin (BSA), hemoglobin, and the Dulbecco's Modified Eagle Medium (DMEM) with 10% fetal bovine serum (FBS) (FIGS. 2f and S17). The fluorescence intensity of EGTP was clearly enhanced selectively with TMPRSS2. While Trypsin can cause some fluorescence enhancement due to its cleavage after Arginine (R) of the peptide sequence. And Camostat can result in significantly reduced fluorescence of EGTP with TMPRSS2 incubation (FIG. 2g). The decreased fluorescence intensity is linear correlated with the concentrations of Camostat (FIG. 2h). The results demonstrated that EGTP can detect recombinant TMPRSS2 and TMPRSS2 inhibitors in buffer or cell culture medium.

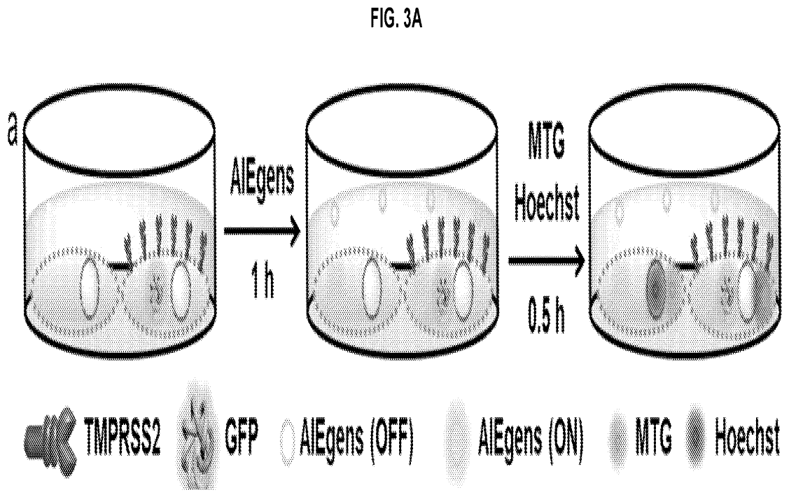

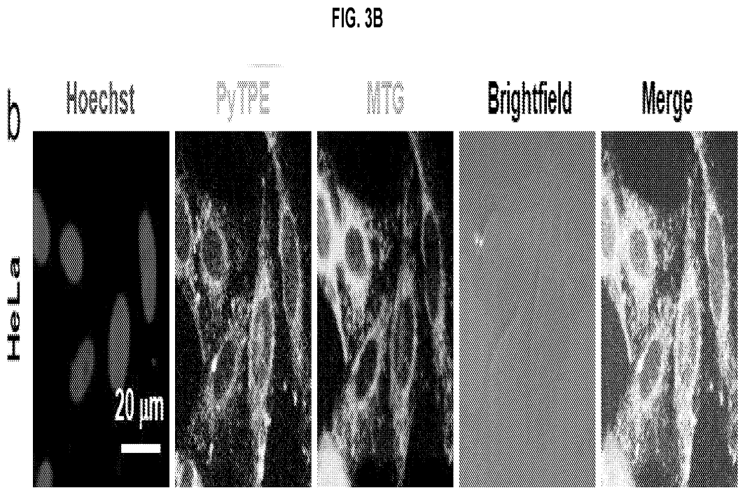

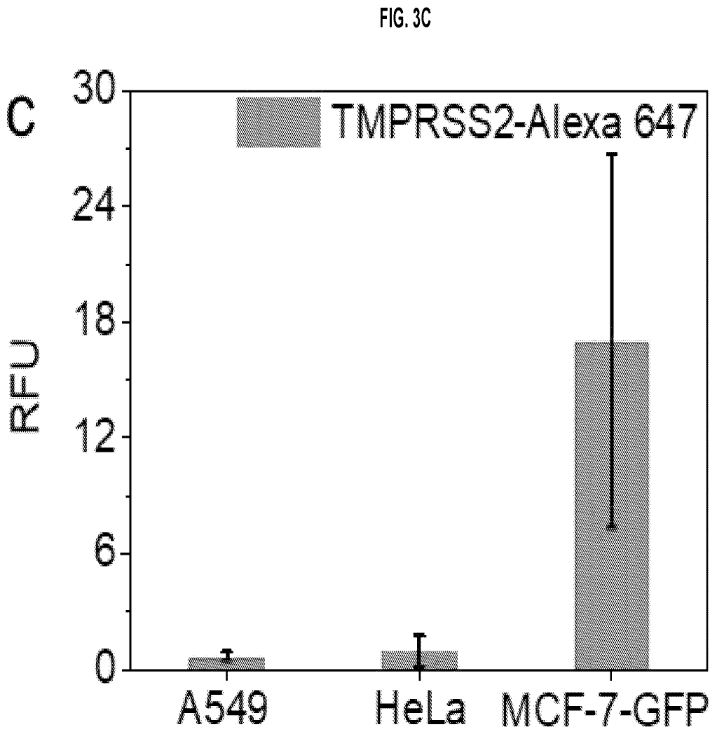

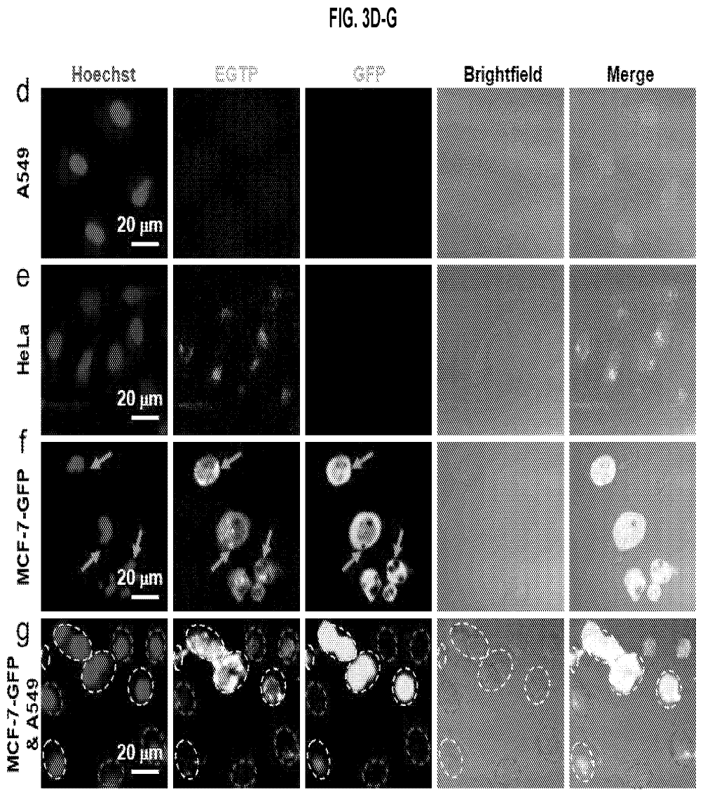

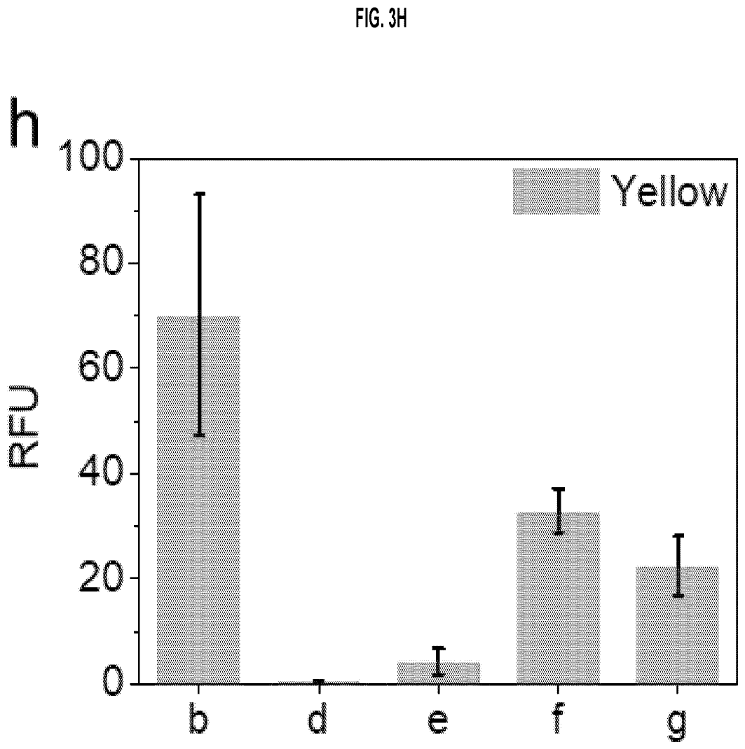

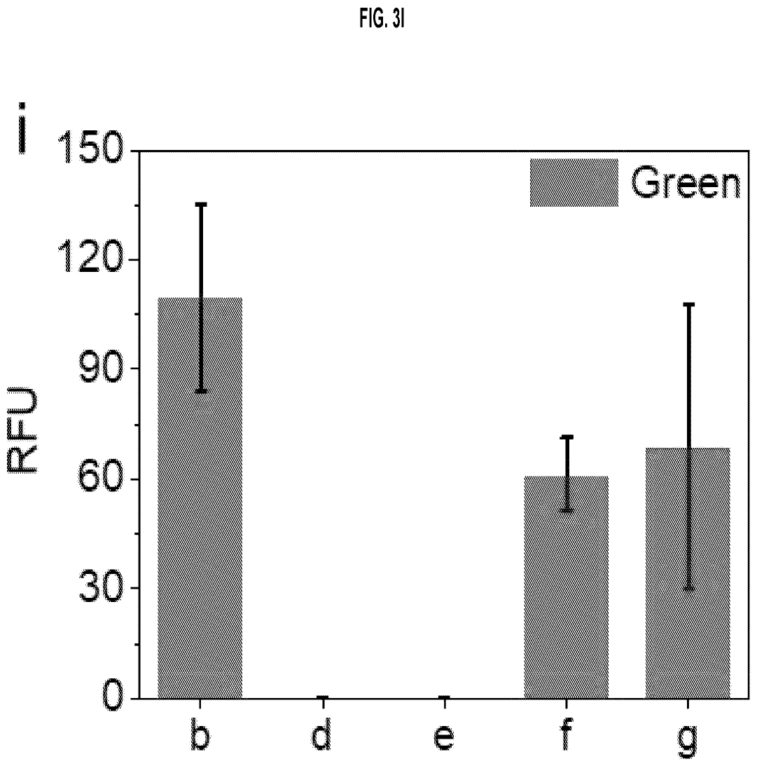

TMPRSS2 selective imaging in cancer cells with EGTP. The TMPRSS2 selective imaging capability of EGTP was tested via the three different cell lines. A549 cells and HeLa cells as TMPRSS2 low expression cell lines, and MCF-7-GFP cells (stable expression of GFP in order to distinguish from other cell lines) as TMPRSS2 high expression cell line were co-cultured with PyTPE, EGTP, TGP, EGTP, Hoechst 33258 (nucleus staining), Mito tracker green (MTG), and Alexa Fluor 647 (Alexa 647) labeled TMPRSS2 antibody for confocal laser scanning microscopy (CLSM) observation (FIG. 3a). First, we confirmed that the yellow fluorescence of PyTPE overlapped well with the green fluorescence of MTG in the HeLa cells due to the hydrophobic effect and electrostatic interactions of positively charged pyridinium units and hydrophobic alkyl chain from PyTPE (FIG. 3b). We then used immunofluorescence imaging to validate the TMPRSS2 relative expression among these three cell lines (FIGS. 3c and S18), which was similar to the previously reported data. In addition, to examine the cytotoxicity and optimized concentration of each probes for cell imaging, different concentrations between 1 μM and 20 μM of PyTPE, EGT, EGTP, TGP, and EGEP were incubated with A549 cells, HeLa cells, and MCF-7-GFP cells for 48 h under standard cell culture conditions (Figure S19). EGT and EGEP showed negligible toxicity to these cells with almost 100% cell viability at the tested concentrations. While high concentration of PyTPE (greater than (>) 5 μM), EGTP (>10 μM), and TGP (>10 μM) can cause significant cytotoxicity. According to the CLSM images of MCF-7-GFP cells, versus the fluorescence of 5 μM EGTP incubation, the stronger yellow fluorescence of EGTP appeared at higher concentrations (10 μM) (Figure S20). Therefore, 10 μM EGTP was used for TMPRSS2 selective imaging for cell imaging experiments.

Under the same staining condition, A549 cells showed almost no yellow fluorescence (FIG. 3d). Only weak yellow fluorescence was displayed in the HeLa cells (FIG. 3e). And strong green and yellow fluorescence was both observed in the MCF-7-GFP cells (FIG. 3f). Especially, in the MCF-7-GFP and A549 co-cultured cells, only MCF-7-GFP cells (white dotted ring) produced strong green and yellow fluorescence signal rather than A549 cells (red dotted ring), thus indicating that TMPRSS2 can distinguish TMPRSS2 highly expressed cell lines (FIGS. 3g, 3h and 3i). While A549 cells, HeLa cells, and MCF-7-GFP cells all showed strong yellow fluorescence with TGP incubation due to the lack of polyglutamic acid and nonspecific internalization by positive charged units (Figure. S21). And few yellow fluorescence observed in the MCF-7-GFP cells and MCF-7-GFP and A549 co-cultured cells with EGEP incubation because of no cleavage of polyglutamic acid (Figure S22). These data verified that EGTP rather than TGP and EGEP can detect TMPRSS2 expression among different cell lines.

FIG. 3. TMPRSS2 selective imaging in cancer cells with EGTP. (a) The experimental scheme of TMPRSS2 slelective imaging between TMPRSS2 low and high expression cells incubation with EGTP. Confocal laser scanning microscopy (CLSM) images of (b) HeLa cells with PyTPE and MTG, and (d) A549 cells, (e) HeLa cells, (f) MCF-7-GFP cells, and (g) MCF-7-GFP & A549 co-cultured cells with EGTP. The average fluorescence intensities of (c) A549 cells, HeLa cells, and MCF-7-GFP cells with Alexa 647 labeled TMPRSS2 antibodies, (h) yellow fluorescence, and (e) green fluorescence in panels b, d, e, f, and g. The MCF-7-FGP cells is activated in the green channel. The EGTP is activated in the yellow channel when cells express TMPRSS2. The pink arrows show clear fluorescence only from EGTP. In panel g, the white and red dotted rings denote the MCF-7-GFP cells and A549 cells, respectively.

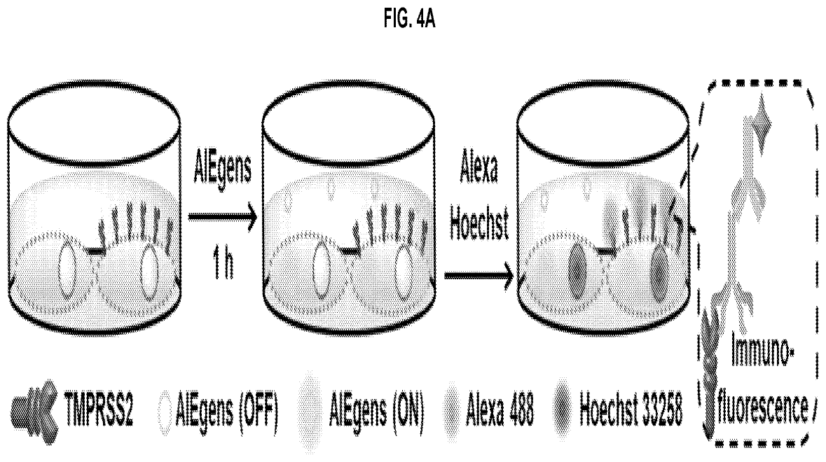

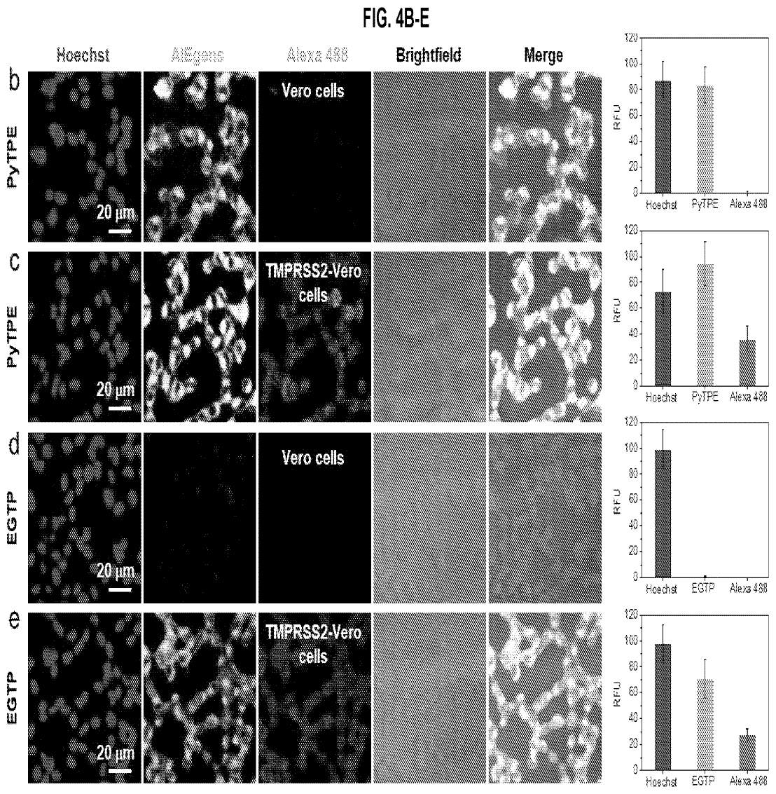

FIG. 4. Validation of EGTP in the Vero cells and TMPRSS2-Vero cells. (a) The experimental scheme of Vero cells and TMPRSS2-Vero cells after incubation with different AIEgens. The enlarged portion shows the imaging mechanism of immunofluorescence imaging. Confocal laser scanning microscopy (CLSM) images and the relative fluorescence intensities of the Vero cells with (b) PyTPE and (d) EGTP incubation, and TMPRSS2-Vero cells with (c) PyTPE and (e) EGTP incubation. The EGTP and Alexa 488 are activated in the yellow and green channel when cells express TMPRSS2.

TMPRSS2 selective imaging and inhibition of Vero cells and TMPRSS2-Vero cells with EGTP and Camostat. To further screen TMPRSS2 inhibitors with EGTP, Vero cells and TMPRSS2-Vero cells as robust cell models were chosen for cell imaging (FIG. 4a). Vero cells and TMPRSS2-Vero cells were incubated with different concentrations (1 mM, 5 mM, 10 mM, and 20 mM) of PyTPE and EGTP (Figures S23, S24, S25, and S26). The expression of TMPRSS2 in Vero cells and TMPRSS2-Vero cells was confirmed by Alexa Fluor 488 (Alexa 488) labeled TMPRSS2 antibodies (FIGS. 4b and 4c). The yellow fluorescence of PyTPE enhanced with increasing concentrations both in the Vero cells and TMPRSS2-Vero cells. While the obvious yellow fluorescence of EGTP was only observed in the TMPRSS2-Vero cells with more than 10 mM EGTP incubation (FIGS. 4d and 4e). After validating that EGTP could selectively image TMPRSS2 in the TMPRSS2-Vero cells, we then tested whether EGTP could use for accurate TMPRSS2 inhibitor screening (FIG. 5a). Thus, TMPRSS2-Vero cells were incubated with different concentrations (0 mM, 10 mM, 100 mM, and 1000 mM) of Camostat before adding the EGTP, and HOECHST 33258™ (FIGS. 5b, 5c, 5d, and 5e). The yellow fluorescence of EGTP gradually decreased with the increaing concentrations of Camostat incubation. While there was no significant effect on the fluorescence intensity of EGTP with GC376 and GRL0617 incubation (Figure S27). These data confirmed that EGTP can be used for TMPRSS2 inhibitors accurate screening in living cells.

FIG. 5. Accurate TMPRSS2 inhibitor screening with EGTP and Camostat. (a) The experimental scheme of TMPRSS2-Vero cells after incubation with Camostat and EGTP. Confocal laser scanning microscopy (CLSM) images and the relative fluorescence intensities of TMPRSS2-Vero cells with different concentrations of Camostat and EGTP for cell imaging. (b) 0 mM Camostat, (c) 10 mM Camostat, (d) 100 mM Camostat, and (e) 1000 mM Camostat. The EGTP is activated in the yellow channel when cells express TMPRSS2 without Camostat inhibition.

FIG. 7: A protease-responsive and surface-potential-tunable peptide-conjugated AIEgen (EGTP) for TMPRSS2 selective imaging and accurate inhibitor screening in living cells is presented. Combining with mitifunctional peptides and AIEgens, the rational construction, catalytic efficiency, structural and surface-potential change, and intracellular distribution of EGTP are exploited with recombinant TMPRSS2, the MCF-7-GFP cells, TMPRSS2-Vero cells and TMPRSS2 inhibitors.

### Conclusions

In summary, this work exploited a TMPRSS2-responsive and surface-potential-tunable peptide-conjugated probe (EGTP) with mitifunctional peptides and AIEgen for selective imaging of TMPRSS2 in the MCF-7-GFP cells and TMPRSS2-Vero cells. We utilized the positive charged PyTPE to realize both cell internalization and cell imaging, the negative charged polyglutamic acid to block the internalization, and the TMPRSS2-responsive sequence to regulate the internalization and aggregation of PyTPE. Importantly, the delivery process and AIE effect of EGTP were controlled by the rational composition of the modular-peptides. Versus control probes without negative charged polyglutamic acid (TGP) and TMPRSS2-responsive sequence (EGEP), EGTP can be effectively cleaved by TMPRSS2 and form aggregates with bright yellow fluorescence. We verified the TMPRSS2 detection and accurate inhibitor screening property of EGTP with three different cancer cell lines, Vero cells, TMPRSS2-Vero cells, and Camostat. This strategy for protease-responsive and surface-potential-tunable peptide-conjugated probes provides for effective membrane protein imaging and is an accurate inhibitor screening agent for precise diagnosis and treatment of cancer and SARS-CoV-2.

### Materials and Methods

Chemistry methods and characterization. The synthesis protocols and details are provided in the supplementary information.

Enzymatic assay with EGTP, TGP, and EGEP. The stock solution of PyTPE, EGTP, TGP, and EGEP in 20 mM Tris-HCl buffer (pH=8.0) was diluted with TMPRSS2 assay buffer (20 mM Tris-HCl buffer (pH 8.0) with 150 mM NaCl, 1 mM DTT, and 5% glycerol) to make 1, 5, 10, and 20 μM working solutions. Recombinant TMPRSS2 was added into the working solution and then diluted to a total of 100 μl with the same Tris-HCl buffer. The reaction mixture was incubated at 37° C. for 1 h for UV-Vis absorption and fluorescence measurement. The solution was excited at 405 nm, and the emission was collected from 430 nm to 800 nm.

Cell culture and plasmid transfection. All the cells were cultured in Dulbecco's Modified Eagle Medium (DMEM) with 10% fetal calf serum (FBS) and 1% penicillin streptomycin (PS, 10000 IU penicillin and 1000 μg/mL streptomycin, multicell) in a cell culture plates at 37° C. in a humidified atmosphere containing 5% CO₂.

Incubation living cells with probes. For confocal laser scanning microscopy imaging, A549 cells, HeLa cells, MCF-7-GFP cells, Vero cells, and TMPRSS2-Vero cells were seeded into cell culture dishes at a density of 2.0×10⁵ in growth medium (DMEM supplemented with 10% FBS, 200 mL). After overnight incubation, the cells were washed with phosphate-buffered saline (PBS, pH 7.4) three times. A solution of the indicated probe in PBS was then added, and the cells were incubated in a 5% CO₂ atmosphere at 37° C. for further use. Hoechst 33258 and MTG (or ALEXA FLUOR 488™ or ALEXA FLUOR 647™) were subsequently added for probe incubation. The supernatant was then discarded, and the cells were washed gently twice with PBS and fixed with 2% paraformaldehyde (PFA) at room temperature for 20 min prior to optical imaging.

Immunofluorescence. Fixed cells were washed with PBS and then with PBS including 1% BSA and 0.1% TRITON X-100™. Cells were incubated with primary antibody against TMPRSS2 (1:100, abcam) in PBS including 1% BSA and 0.1% TRITON X-100™ overnight at 4° C. Cells were washed and incubated with ALEXA 488™ or ALEXA 647™ goat anti-rabbit secondary antibody (abcam) in PBS including 1% BSA for 1 h at room temperature followed by 3×PBS washes.

Confocal laser scanning microscopy. The fluorescence signals of cells were detected using a LSM880™ confocal laser scanning microscope (Zeiss), equipped with a 63/1.42 numerical aperture oil-immersion objective lens. A 405-nm laser was chosen for the excitation of Hoechst 33258™, the emission was collected at 420-460 nm. A 405 nm laser was chosen for the excitation of AIEgens and the emission was collected at 550-620 nm. A 488 nm laser was chosen for the excitation of GFP (or MTG), the emission was collected at 500-530 nm. A 488 nm laser was chosen for the excitation of ALEXAFLUOR 488™, the emission was collected at 500-550 nm. All fluorescence images were analyzed with an IMAGE™ software (Zeiss).

Cytotoxicity assay. The cytotoxic potential of PyTPE, EGT, EGTP, TGP, and EGEP were assessed using the A549 cells, HeLa cells, and MCF-7 cells for 48 h incubation in quadruplicate in a 96-well plate. The fluorescence of Resazurin solution at 690 nm using an excitation wavelength of 560 nm was recorded by a SYNERGY H1™ microplate reader (BioTek) with standard operation procedures.

### Example 2: Protease-Responsive Surface-Potential-Tunable Peptide-Conjugated AIEgens for TMPRSS2 Selective Imaging and Accurate Inhibitor Screening

This example describes making and using protease-responsive and surface-potential-tunable peptide-conjugated AIEgens (EGTP) for TMPRSS2 selective imaging and accurate inhibitor screening as provided herein.

Rink-amide resin (particle size 100 to 150 mesh; loading 0.67 mmol/g), Fmoc-protected L-amino acids, and O-benzotriazole-N, N, N′, N′-tetramethyl-uronium-hexafluorophosphate (HBTU) were purchased from AAPPTec, LLC. (Kentucky, USA). The 4-(1,2,2-triphenylvinyl) benzaldehyde, 1,4-dibromobutane, 4-methylpyrdine, HOECHST 33258™, and propidium iodide (PI) were purchased from Combi-Blocks, Inc. (San Diego, USA). Tris-(hydroxymethyl) aminomethane (Tris-base), tris-(hydroxymethyl) aminomethane hydrochlorideand (Tris-HCl), sodium azide, sodium ascorbate, copper (I) iodide, N, N-diisopropylethylamine (DIPEA), thrombin, hemoglobin, albumin bovine serum, lipopolysaccharide, and resazurin based in vitro toxicology assay kit were purchased from Sigma-Aldrich (Missouri, USA). All other reagents were obtained from commercial sources and used without further purification. Deionized water (18.2 MΩ·cm) used in all experiments was purified with a Milli-Q Academic water purification system (Millipore Corp., Billerica, MA, USA).

Peptides were synthesized using an AAPPTEC ECLIPSE™ system through standard solid phase Fmoc syntheses on rink-amide resin. Electrospray ionization mass spectrometry (ESI-MS) data was acquired by using a MICROMASS QUATTRO ULTIMA™ mass spectrometer (Waters Corp.). High resolution mass spectra (HRMS) were recorded on a MICROTOF II™ mass spectrometer system (Bruker). High performance liquid chromatography (HPLC) was performed by using a LC-40™ HPLC system (Shimadzu) under the wavelength of 195 nm, 254 nm, and 405 nm. The sample was dissolved in water or acetonitrile, applied on a ZORBAX 300 BS™ C18 column (5 um, 9.4×250 mm) (Agilent), and eluted at 1.5 mL/min with a 40 min gradient from 10% to 95% solvent B where solvent A is water (0.05% TFA solution) and solvent B is acetonitrile (0.05% TFA solution). All products were purified by HPLC to reach a purity of 90%. UV-Vis absorption and fluorescence spectra were performed on a SYNERGY H1™ microplate reader (BioTek). Confocal laser scanning microscopy images were obtained on a LSM880™ confocal laser scanning microscope (Zeiss). Dynamic light scattering (DLS) measurements were performed on a NANO-ZS90 ZETASIZER™ (Malvern) to determine hydrodynamic sizes and zeta potential values of the nanosystems studied. Transmission electron microscopy (TEM) images were acquired using an FEI TECNAI F20™ instrument at an operation voltage of 200 kV. Flow cytometric analysis was carried out under a flow cytometer BD LSR™ (Fortessa).

### Synthesis and Characterization

(1) PyTPE synthesis: 1, 2 PyTPE was synthesized according to our previous reports and the procedures in the literatures.

(2) Crude peptide synthesis: 100 mg rink amide resin was chosen in solid-phase peptide synthesis. 2×4 mL 20% piperidine in DMF, 0.1 M amino acid in 3 mL DMF (5 equivalents), 0.1 M HBTU in 3 mL DMF (5 equivalents), and 0.2 M DIPEA in 3 mL DMF (10 equivalents) were used for each coupling cycle under the protection of nitrogen. After the completion of solid phase synthesis, peptides were cleaved of the resins by treating with cleavage cocktail containing 2.5% thioanisole, 3% TIPS, 2.5% water, and 92% TFA for 3 hours followed by filtration to remove resin. Peptides were precipitated and centrifuged by addition of ice-cold diethyl ether to the filtrates. The residue was dissolved in 5 mL MeCN/H2O (v/v=1:1) solution and lyophilized. Crude peptides were purified using semi-preparative HPLC.

(3) Coupling with PyTPE: 1, 2 PyTPE (5.0 mg, 7.1 □mol), sodium ascorbate (0.7 mg, 3.6 μmol), copper (I) iodide (0.7 mg, 3.6 μmol), and the purified peptide (3.6 μmol) were dissolved in DMSO/H2O (v/v=1:1) solution, and stirred at 50° C. for 24 h under protection of nitrogen. The crude probes were purified using semi-preparative HPLC.

### Synthesis of PyTPE.

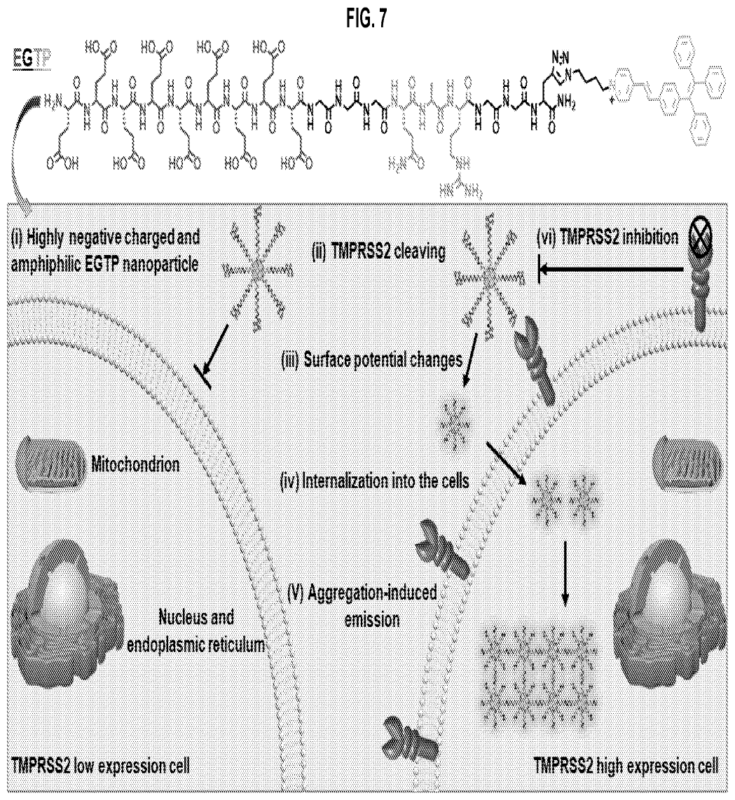

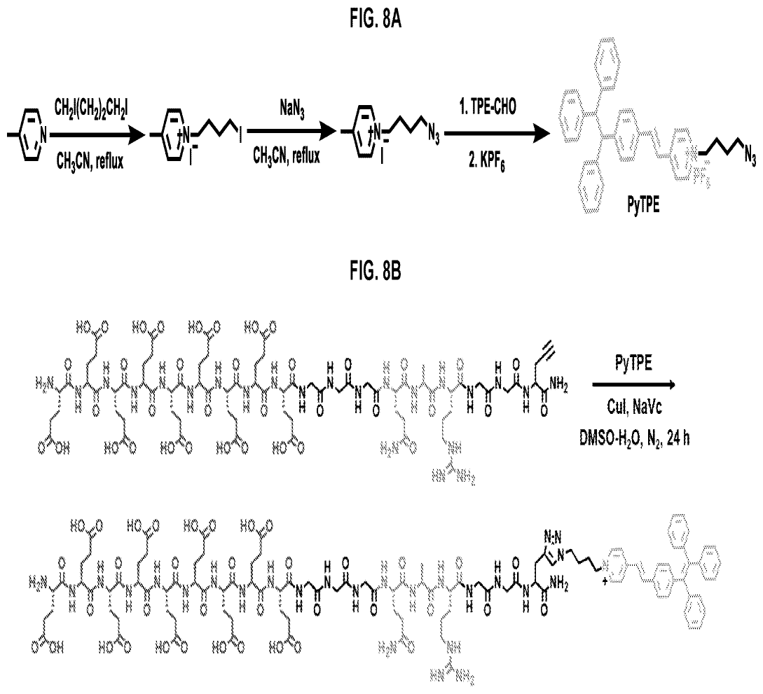

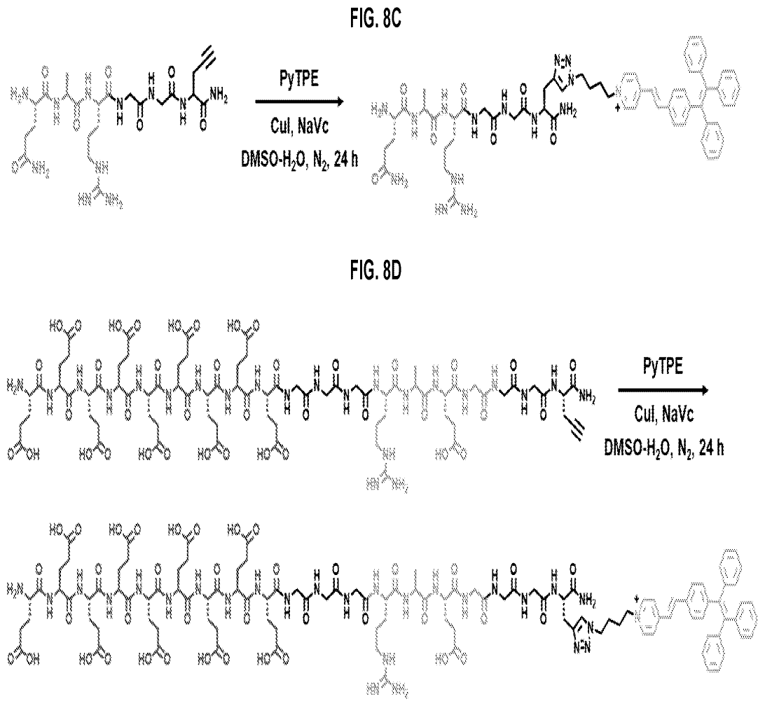

FIG. 8A, Scheme S1. The synthetic route of PyTPE. The main synthetic route of PyTPE is three steps: (a) Synthesis of Py-I. (b) Synthesis of Py-N3, (c) Synthesis of PyTPE, HRMS (ESI) of PyTPE m/z: [M]+ calculated (calcd.) for 533.2700; found, 533.2697.

### Synthesis of EGTP

FIG. 8B, Scheme S2. The synthetic route of EGTP. The main synthetic route of EGTP is two steps through standard solid phase Fmoc synthesis and a copper-catalyzed azide-alkyne click reaction: (a) Synthesis of EGT, ESI-MS (ESI+) m/z: [M+2H]2+/2 calcd. for 957.8820; found, 958.74. [M+3H]3+/3 calcd. for 638.9239; found, 639.22. (b) Synthesis of EGTP, ESI-MS (ESI+) m/z: [M+2H]2+/2 calcd. for 1224.5169; found, 1225.12. [M+3H]3+/3 calcd. for 816.6805; found, 816.90.

### Synthesis of TGP

FIG. 8C, Scheme S3. The synthetic route of TGP. The main synthetic route of TGP is two steps through standard solid phase Fmoc synthesis and a copper-catalyzed azide-alkyne click reaction. (a) Synthesis of TG, ESI-MS (ESI−) m/z: [M−H]1− calcd. for 580.2956; found, 580.47. [M+TFA-H]1− calcd. for 694.2884; found, 694.51. [M+2TFA-H]1− calcd. for 808.2813; found, 808.58. (b) Synthesis of TGP, ESI-MS (ESI−) m/z: [M+TFA-H]1− calcd. for 1227.5584; found, 1228.62. [M+2TFA-H]1-calcd. for 1341.5513; found, 1341.44. [M+3TFA-H]1− calcd. for 1455.5441; found, 1455.50.

### Synthesis of EGEP

### FIG. 8D, Scheme S4. The Synthetic Route of EGEP.

The main synthetic route of EGEP is two steps through standard solid phase Fmoc synthesis and a copper-catalyzed azide-alkyne click reaction. (a) Synthesis of EGE, ESI-MS (ESI−) m/z: [M-H]1− calcd. for 1970.7488; found, 1971.41. [M−2H]2−/2 calcd. for 985.3744; found, 985.61. (b) Synthesis of EGEP, ESI-MS (ESI−) m/z: [M−2H]2−/2 calcd. for 1251.5055; found, 1251.63.

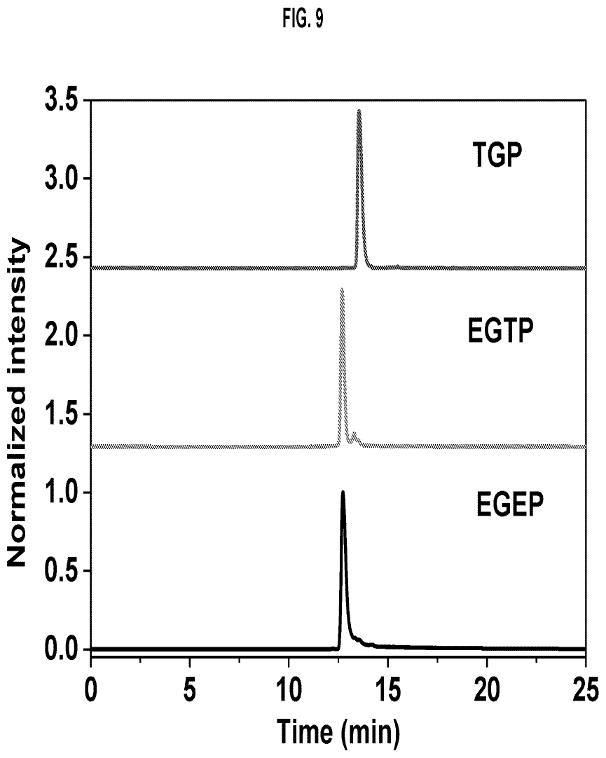

FIG. 9 illustrates HPLC results of TGP, EGTP, and EGEP under the absorption wavelength of 405 nm.

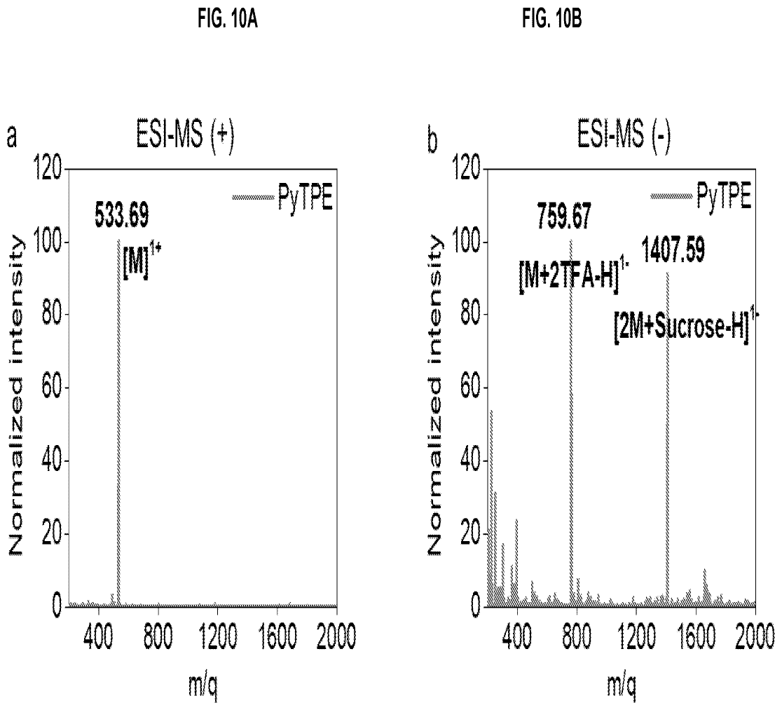

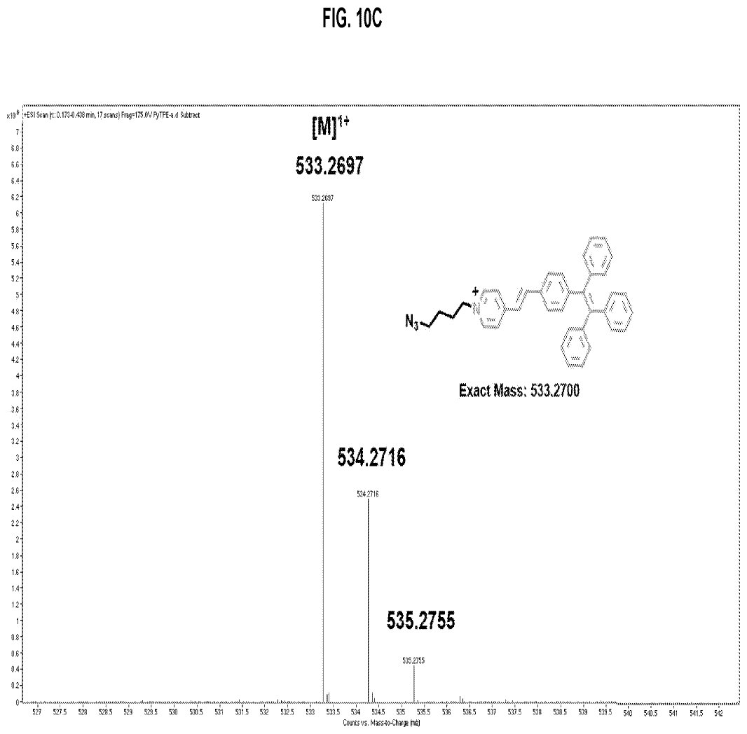

FIG. 10 illustrates (a) The positive mode and (b) negative mode electrospray ionization mass spectrometry (ESI-MS) and (c) high-resolution mass spectrometry (HR-MS) results of PyTPE. [TFA]=114.02, [Sucrose]=342.30.

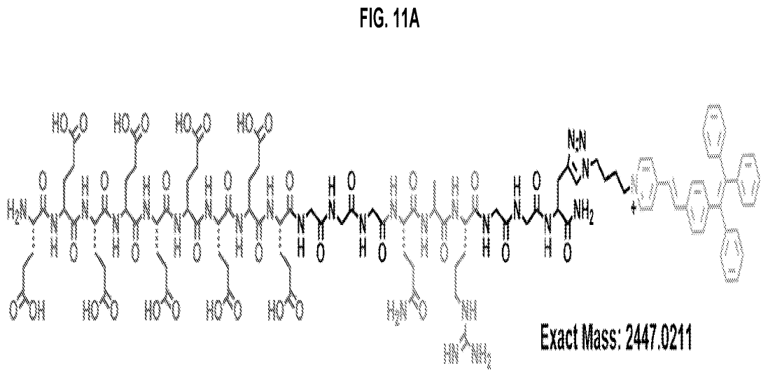

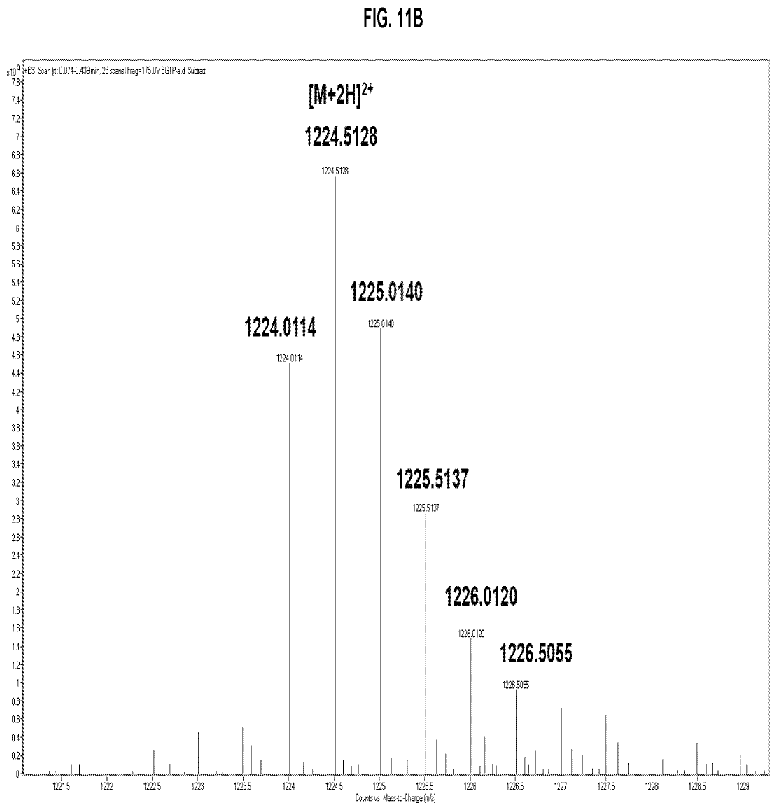

FIG. 11 illustrates the positive mode HRMS results of EGTP with two positive charges.

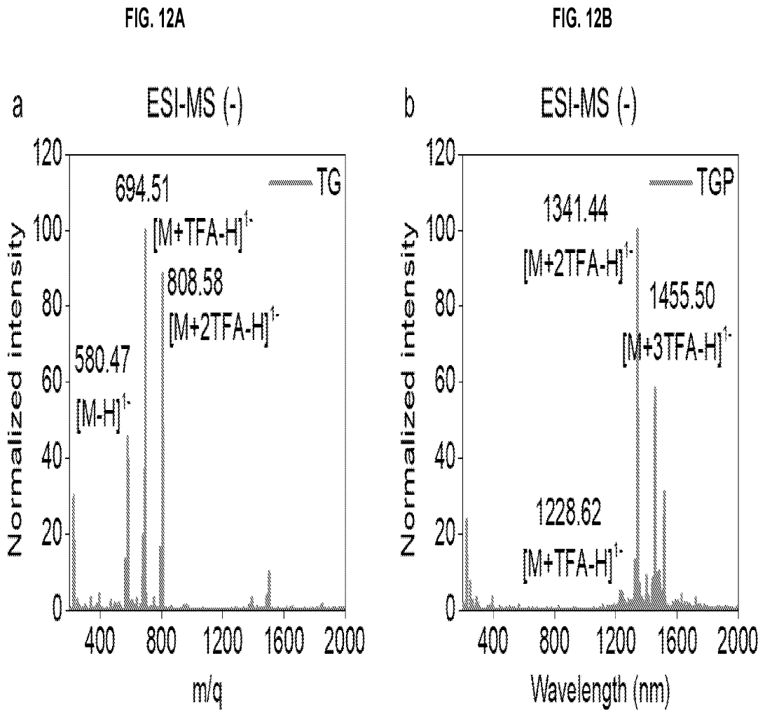

FIG. 12 illustrates the negative mode ESI-MS results of TG and TGP.

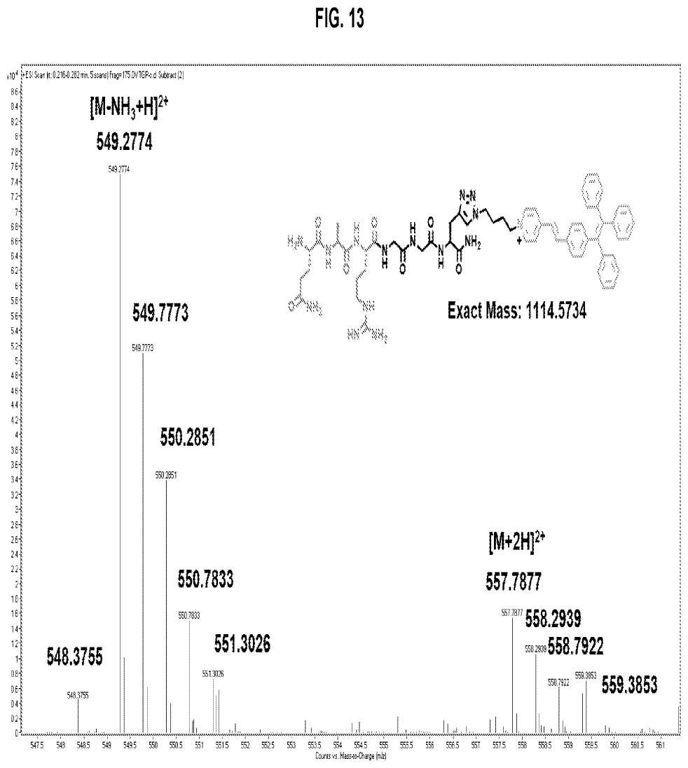

FIG. 13 illustrates the positive mode HRMS results of TGP with two positive charges.

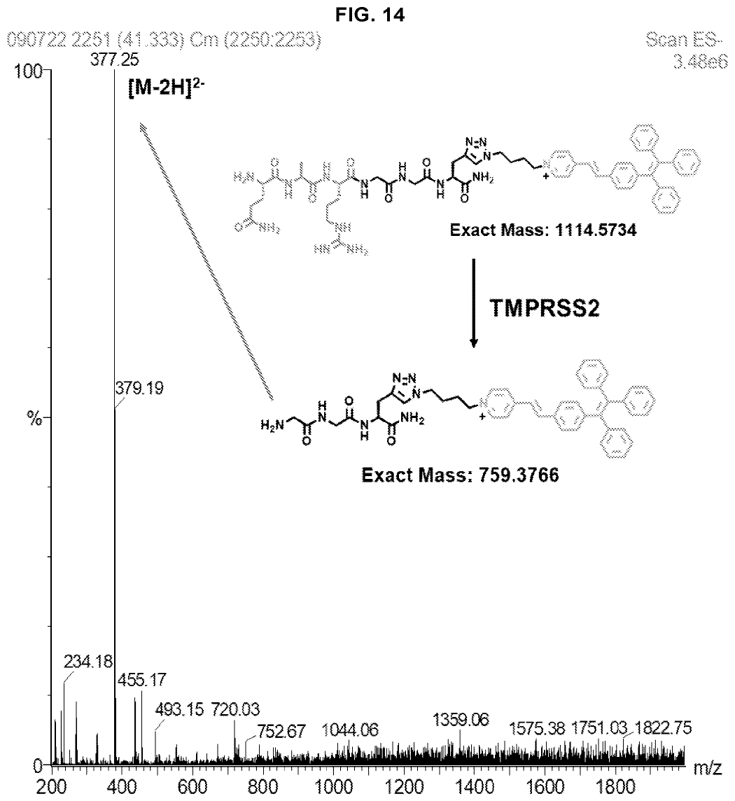

FIG. 14 illustrates the negative mode ESI-MS results of compounds TGP after incubation with TMPRSS2 for 1 h showing that TGP can be cleaved by TMPRSS2.

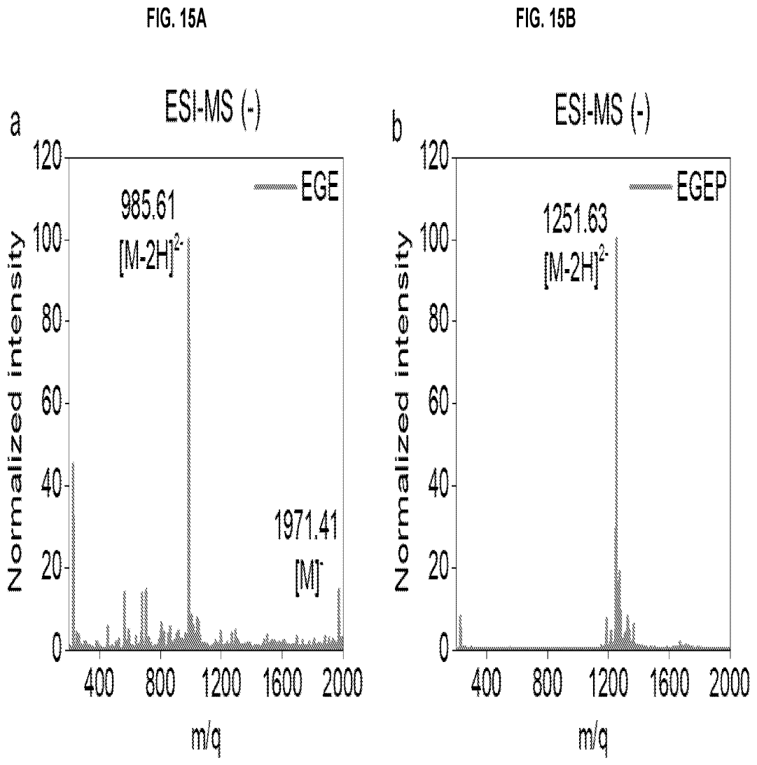

FIG. 15 illustrates the negative mode ESI-MS results of EGE and EGEP.

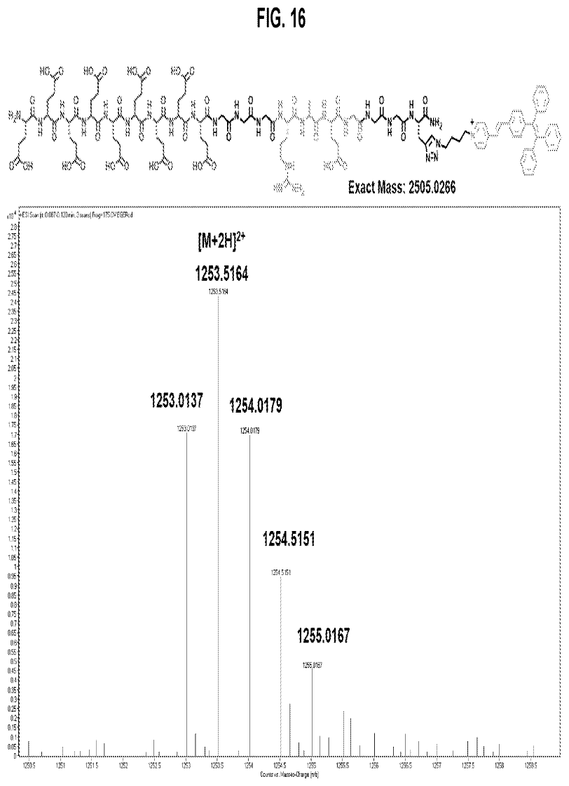

FIG. 16 illustrates the positive mode HRMS results of EGEP with two positive charges.

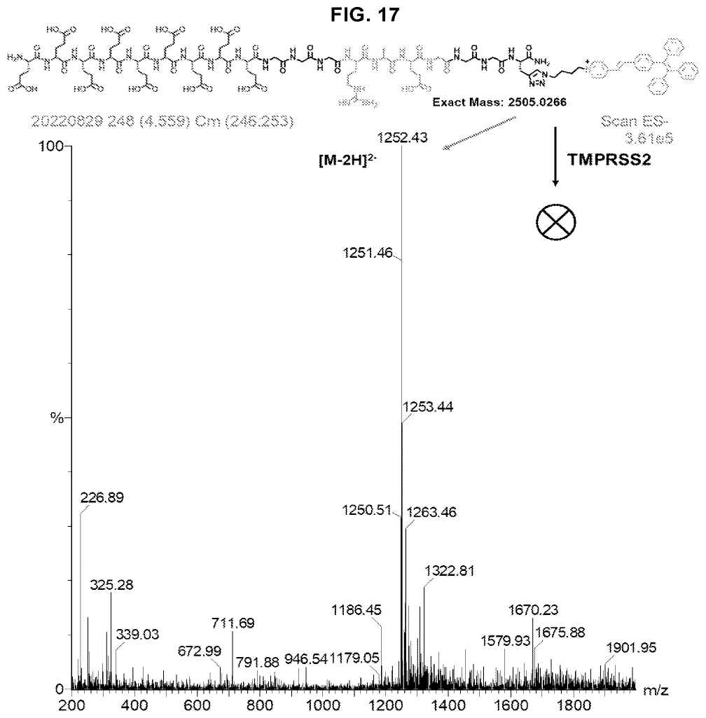

FIG. 17 illustrates the negative mode ESI-MS results of compounds EGEP after incubation with TMPRSS2 for 1 h showing that EGEP cannot be cleaved by TMPRSS2.

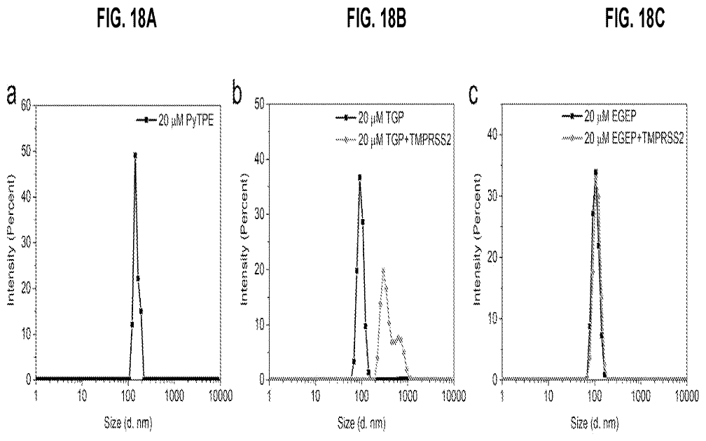

FIG. 18 illustrates the (a) The hydrodynamic size of (a) 20 μM PyTPE, (b) 20 μM TGP and 20 μM TGP with 100 nM TMPRSS2, and (c) 20 μM EGEP and (e) 20 μM EGEP with 100 nM TMPRSS2 in 20 mM Tris-HCl buffer.

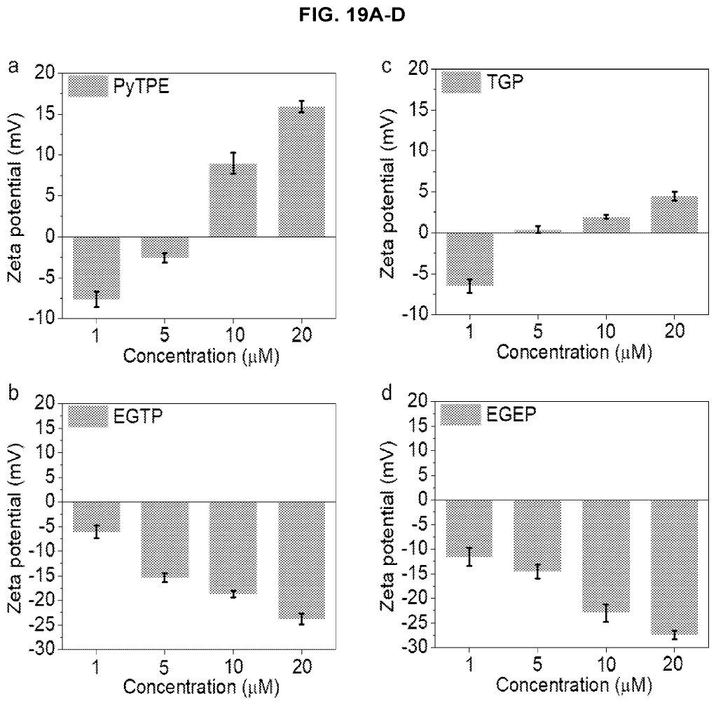

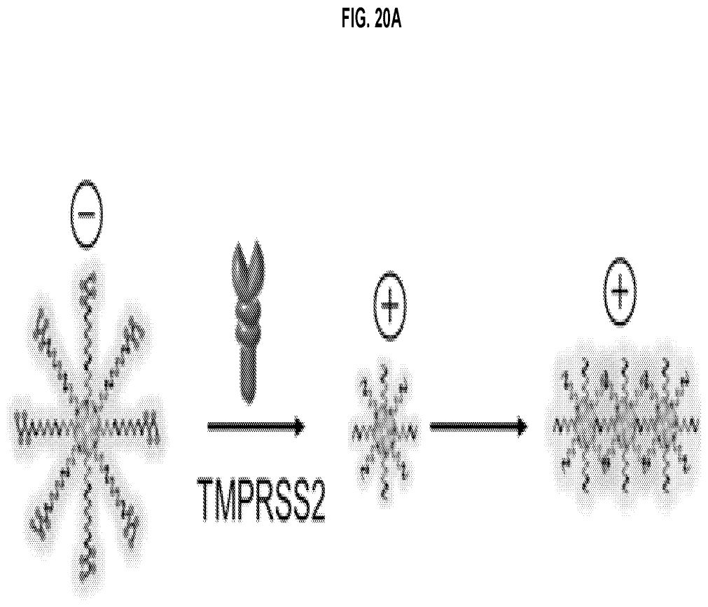

FIG. 19 illustrates Zeta potential values of different concentrations of (a) PyTPE, (b) EGTP, (c) TGP, and (d) EGEP, suggesting good linear change of Zeta potential with increasing concentration of AIEgens.

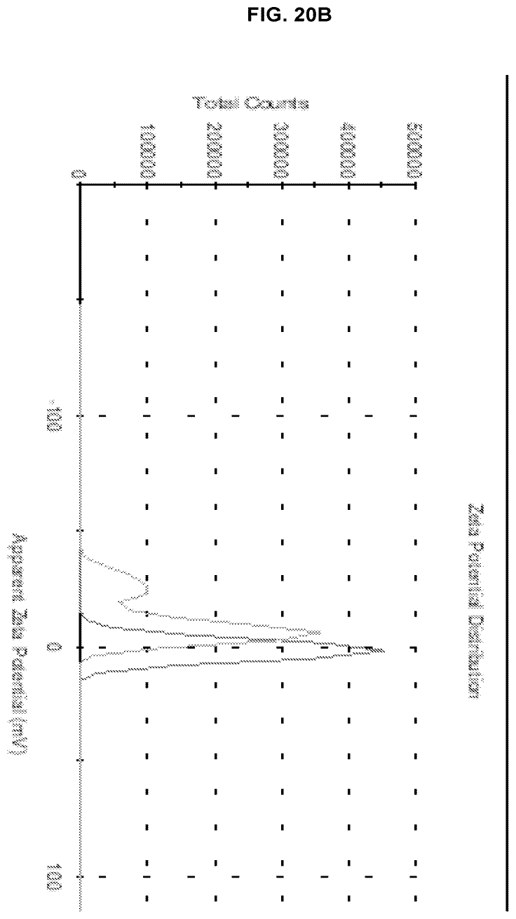

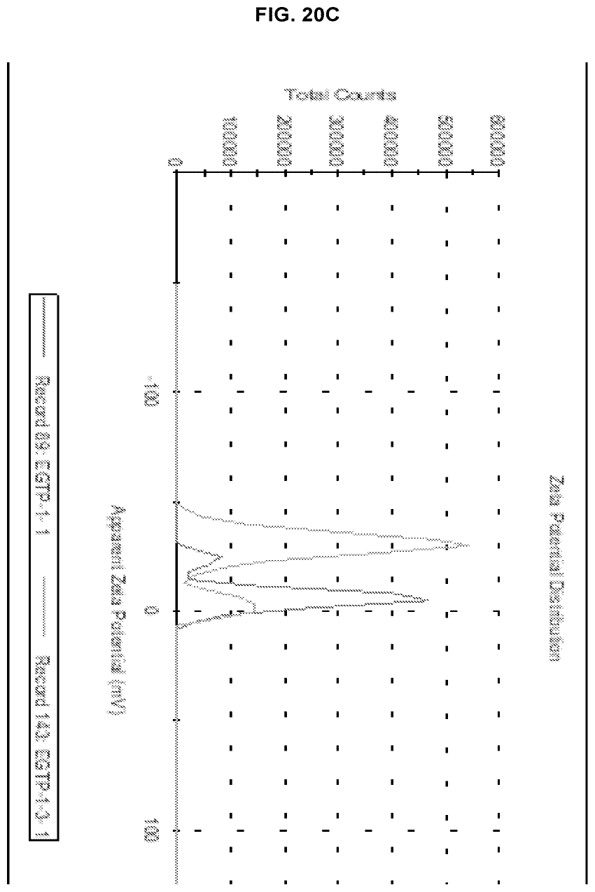

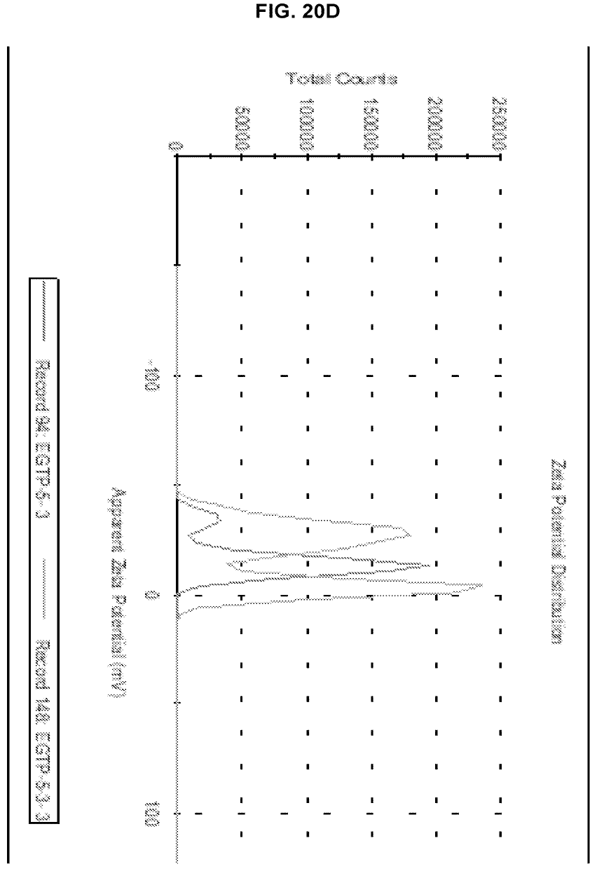

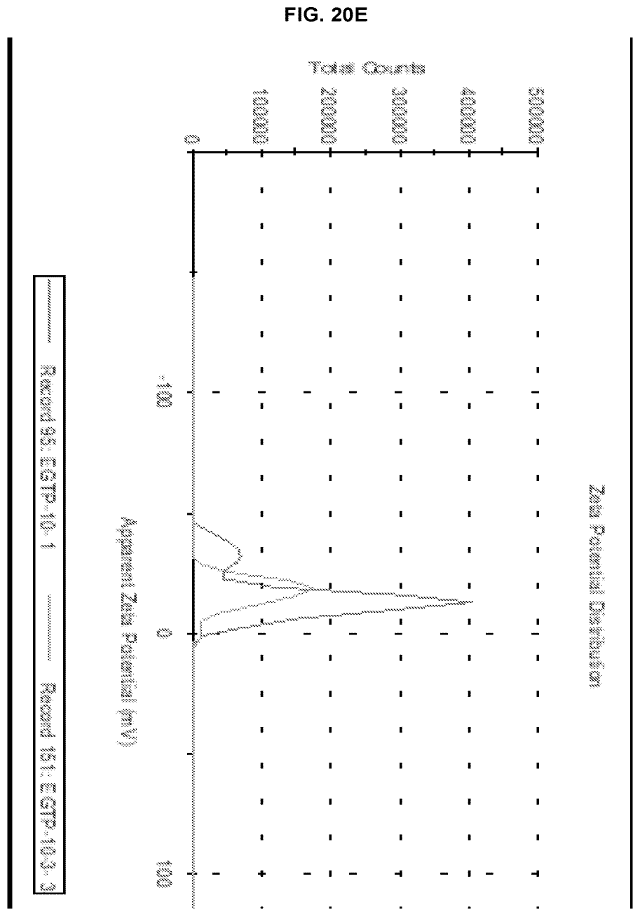

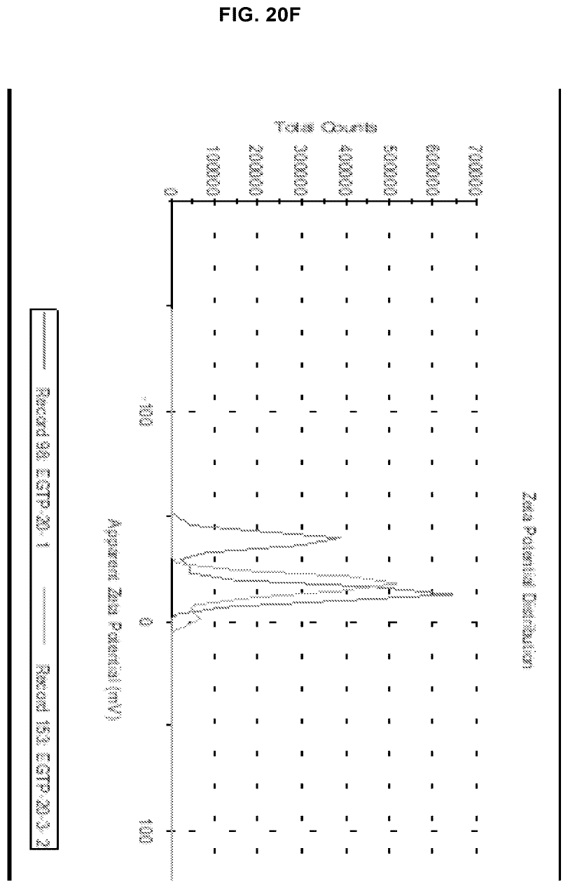

FIG. 20 illustrates Zeta potential values of different concentrations of EGTP with 100 nM TMPRSS2, suggesting the Zeta potential became more positive after incubation with TMPRSS2. (a) Schematic diagram of Zeta potential changes of EGTP with TMPRSS2, (b) Tris-HCl buffer, (c) 1 μM EGTP with/without TMPRSS2, (d) 5 μM EGTP with/without TMPRSS2, (e) 10 μM EGTP with/without TMPRSS2, (f) 20 μM EGTP with/without TMPRSS2.

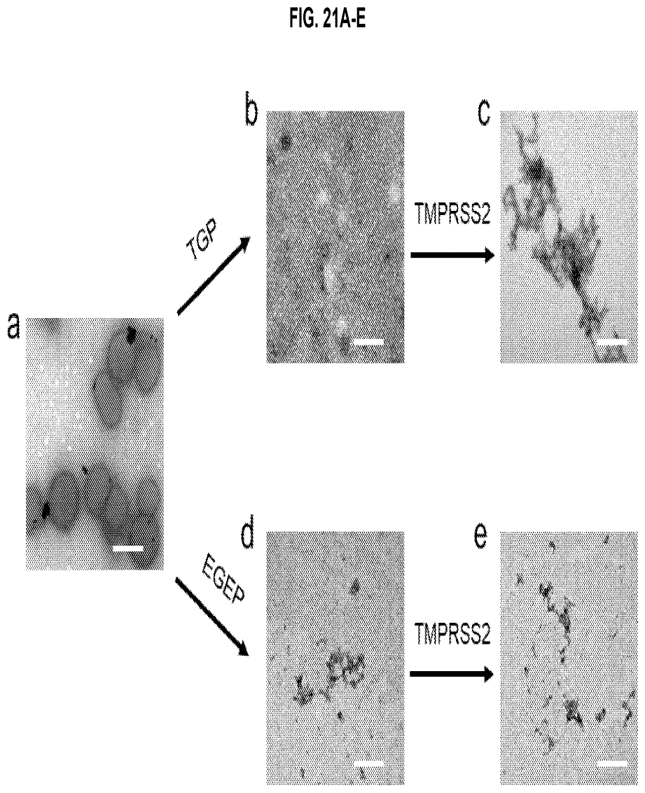

FIG. 21 illustrates transmission electron microscope (TEM) images of (a) 20 μM PyTPE, (b) 20 μM TGP, (c) 20 μM TGP with 100 nM TMPRSS2, (d) 20 μM EGEP, and (e) 20 μM EGEP with 100 nM TMPRSS2 in 20 mM Tris-HCl buffer.

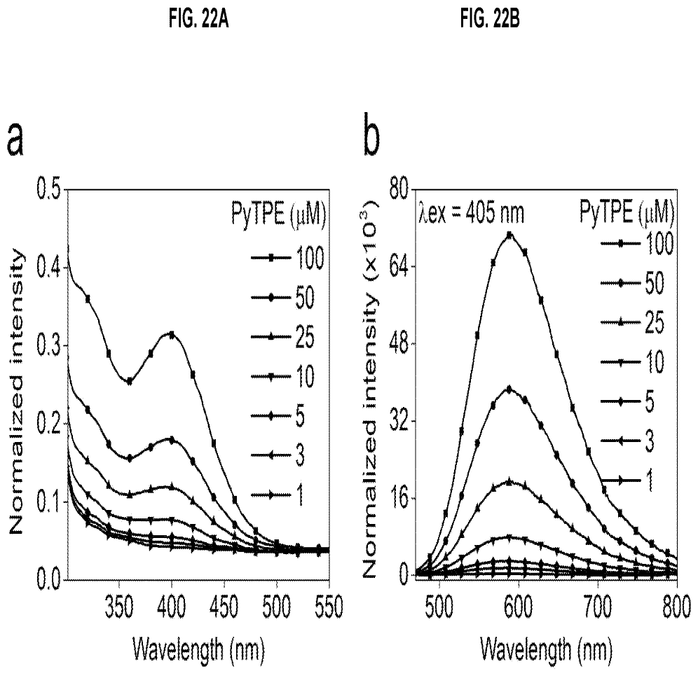

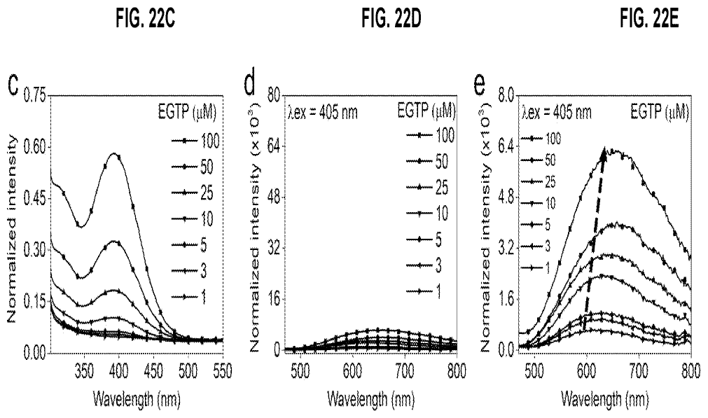

FIG. 22 illustrates (a and c) Absorption, and (b, d, and e) fluorescence spectra of different concentration of PyTPE and EGTP showed the good solubility enhancement with the decreased fluorescence intensity of EGTP in 20 mM Tris-HCl buffer.

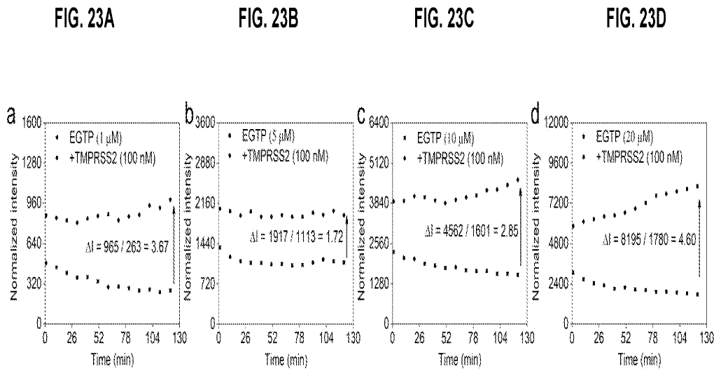

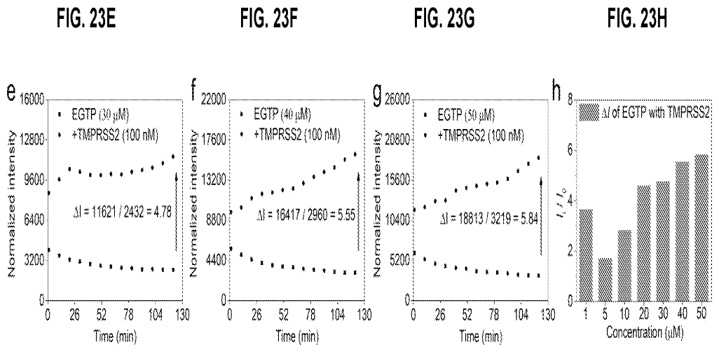

FIG. 23 illustrates Time-dependent fluorescence spectra of different concentration of EGTP incubation with TMPRSS2 in 20 mM Tris-HCl buffer showing that the concentration of EGTP more than 20 μM with TMPRSS2 can display strong fluorescence in 20 mM Tris-HCl buffer.

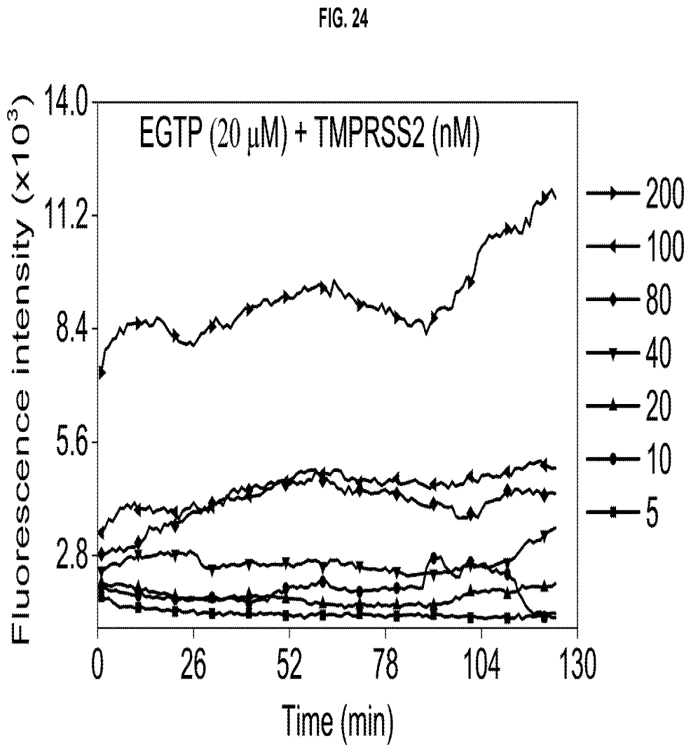

FIG. 24 illustrates Time-dependent fluorescence spectra of 20 μM EGTP incubation with different concentrations of TMPRSS2 in 20 mM Tris-HCl buffer showing that high concentrations of TMPRSS2 can produce strong fluorescence of the same concentration of EGTP.

FIG. 25 illustrates Time-dependent fluorescence spectra of 20 μM EGTP incubation with 20 mM Tris-HCl buffer, DMEM, and DMEM with 10% FBS showing that high concentrations of EGTP can aggregate easily with strong fluorescence in DMEM with 10% FBS.

FIG. 26 illustrates an exemplary experimental scheme, Confocal laser scanning microscopy (CLSM) images and average fluorescence intensities of (b) A549 cells, (c) HeLa cells, (d) MCF-7-GFP cells, and (e) MCF-7-GFP & A549 cells with Alexa 647 labeled TMPRSS2 antibodies and 5 μM Hoechst 33258 for 0.5 h.

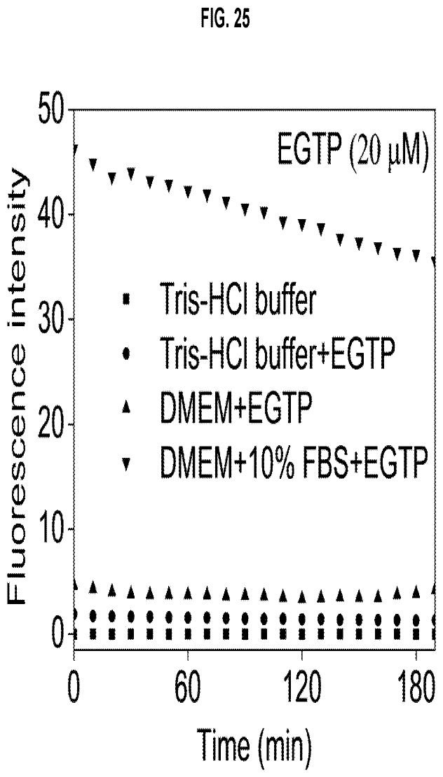

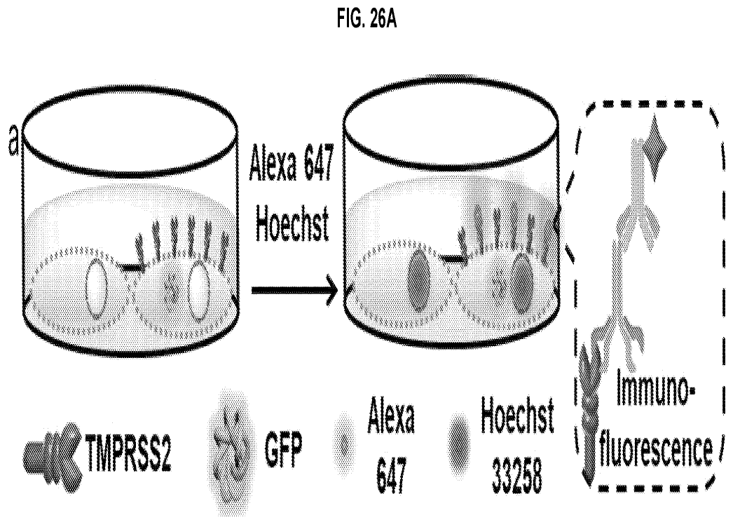

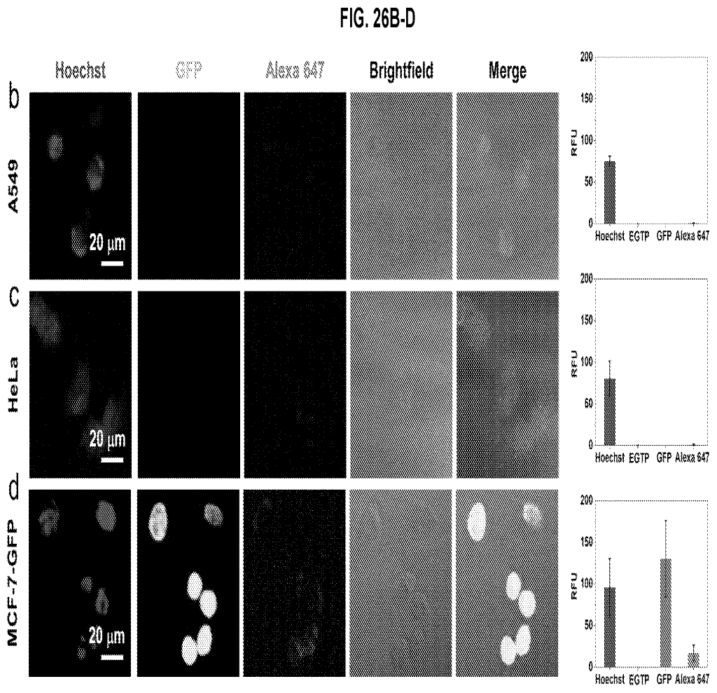

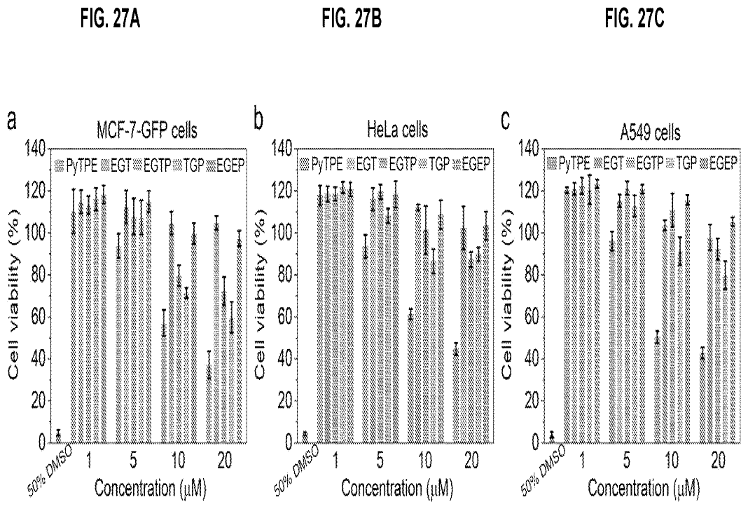

FIG. 27 illustrates metabolic activity of (a) MCF-7-GFP cells, (b) HeLa cells, and (c) A549 cells were studied with (1 μM, 5 μM, 10 μM, and 20 μM) PyTPE, (1 μM, 5 M, 10 μM, and 20 μM) EGT, (1 μM, 5 μM, 10 μM, and 20 μM) EGTP, (1 μM, 5 M, 10 μM, and 20 μM) TGP, (1 μM, 5 μM, 10 μM, and 20 μM) EGEP for 48 h incubation using a fluorescence resazurin assay. The results show that the higher concentration of 10 μM of PyTPE is obviously cytotoxic.

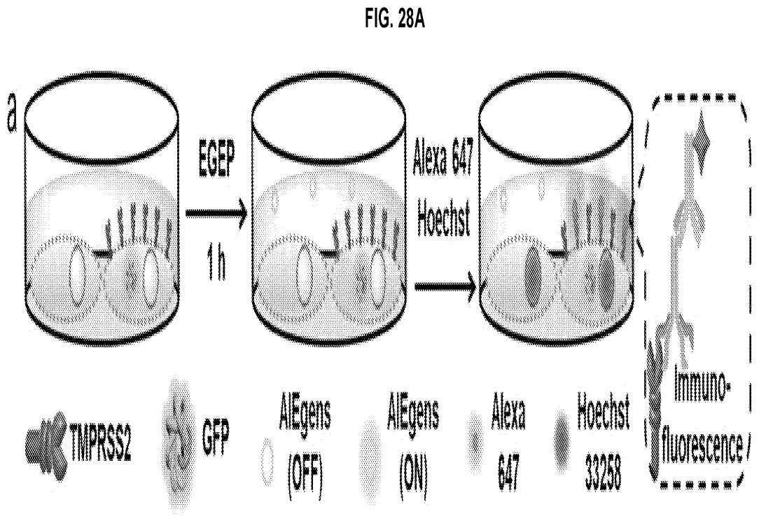

FIG. 28 illustrates (a) The experimental scheme, CLSM images and average fluorescence intensities of (b) MCF-7-GFP cells and (c) MCF-7-GFP & A549 cells with Alexa 647 labeled TMPRSS2 antibodies, 5 μM EGTP for 1 h, and 5 μM Hoechst 33258 for 0.5 h. In panel c, the white and red dotted rings denote the MCF-7-GFP cells and A549 cells, respectively.

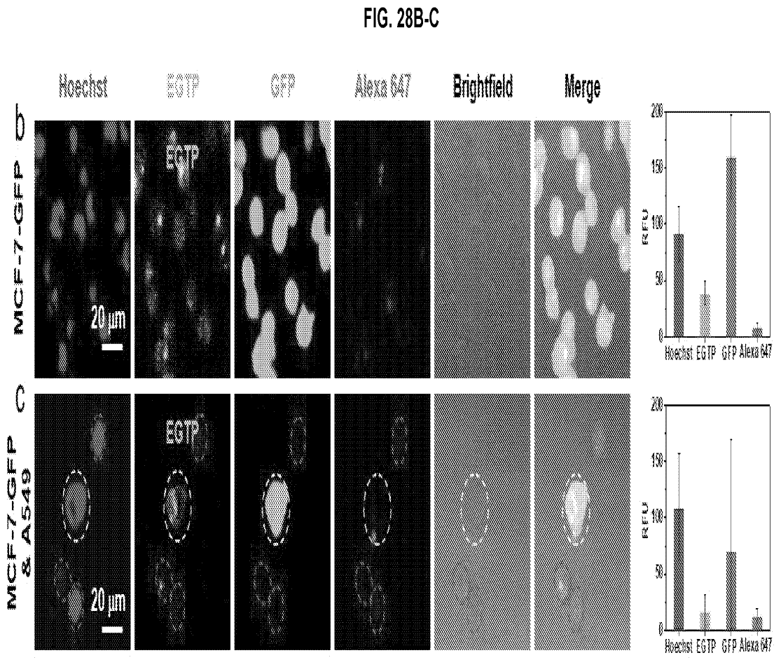

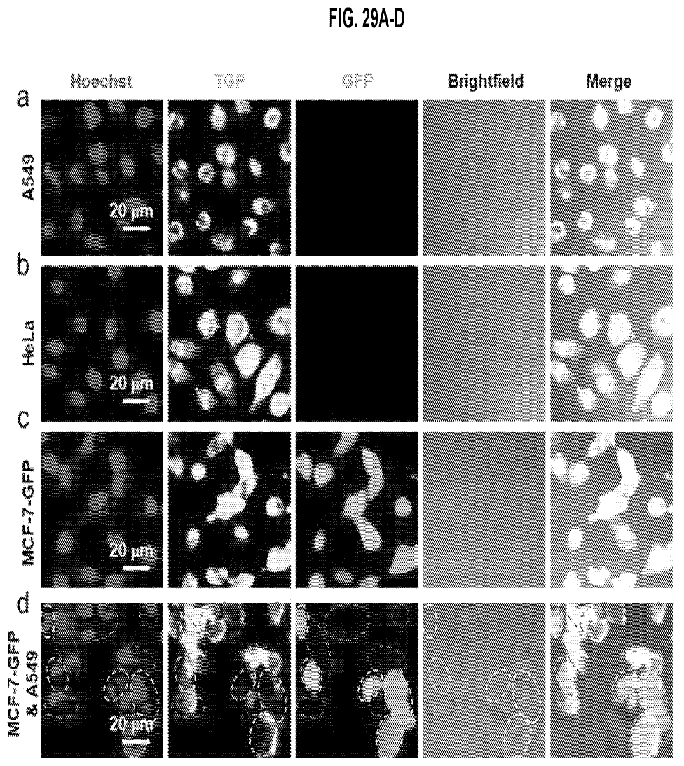

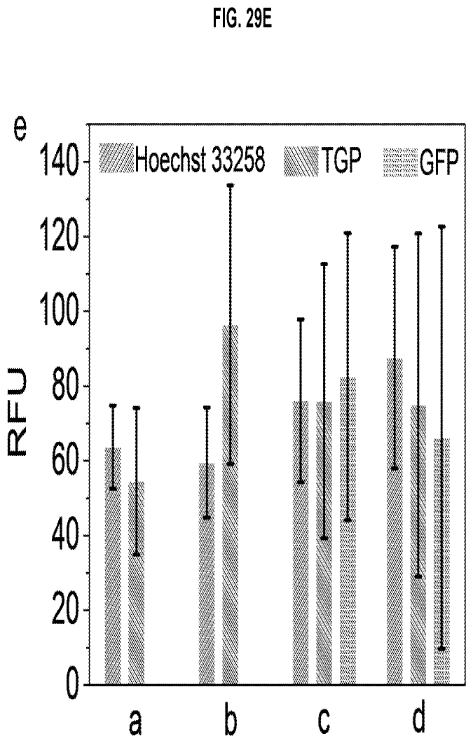

FIG. 29 illustrates CLSM images and average fluorescence intensities of A549 cells, HeLa cells, MCF-7-GFP cells, and MCF-7-GFP & A549 cells with 10 μM TGP for 1 h, and 5 μM Hoechst 33258 for 0.5 h. In panel d, the white and red dotted rings denote the MCF-7-GFP cells and A549 cells, respectively.

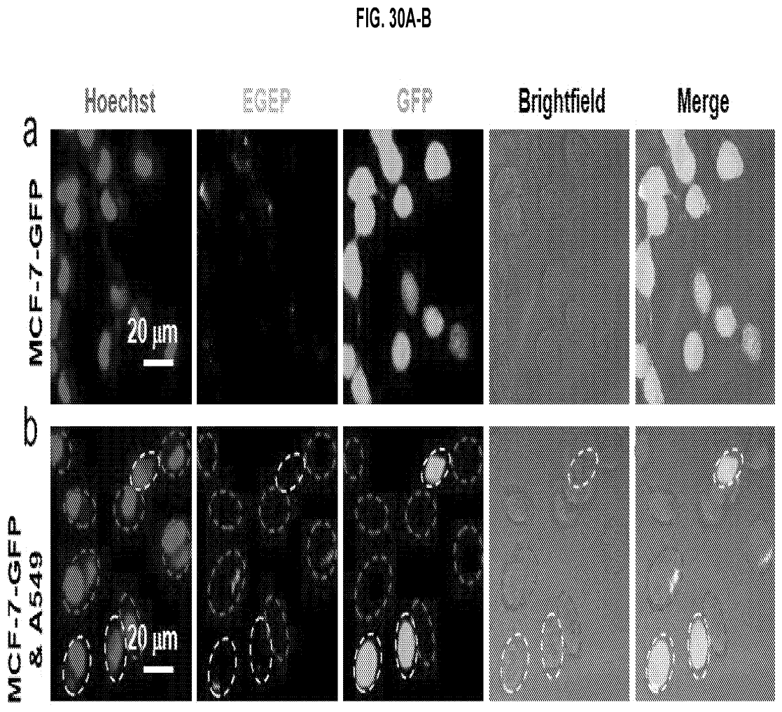

FIG. 30 illustrates CLSM images and average fluorescence intensities of MCF-7-GFP cells and MCF-7-GFP & A549 cells with 10 μM EGEP for 1 h, and 5 μM Hoechst 33258 for 0.5 h. In panel b, the white and red dotted rings denote the MCF-7-GFP cells and A549 cells, respectively.

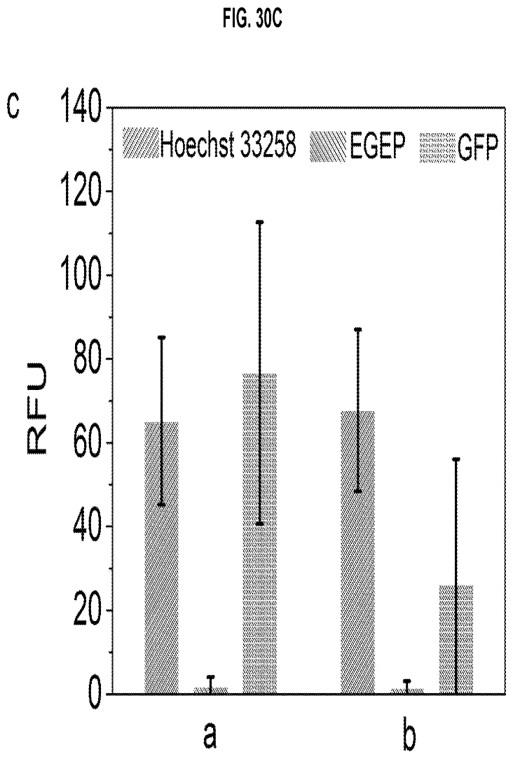

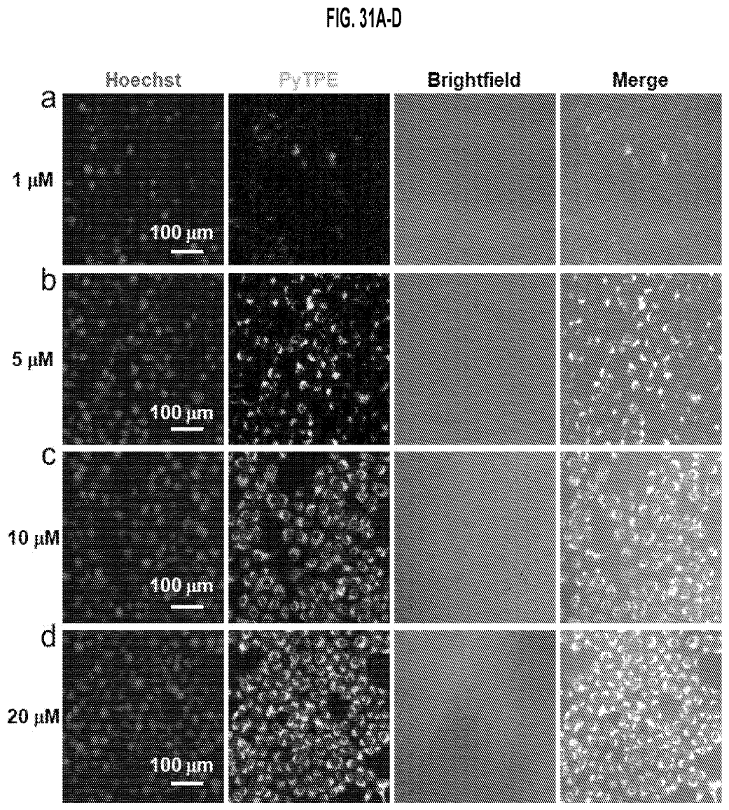

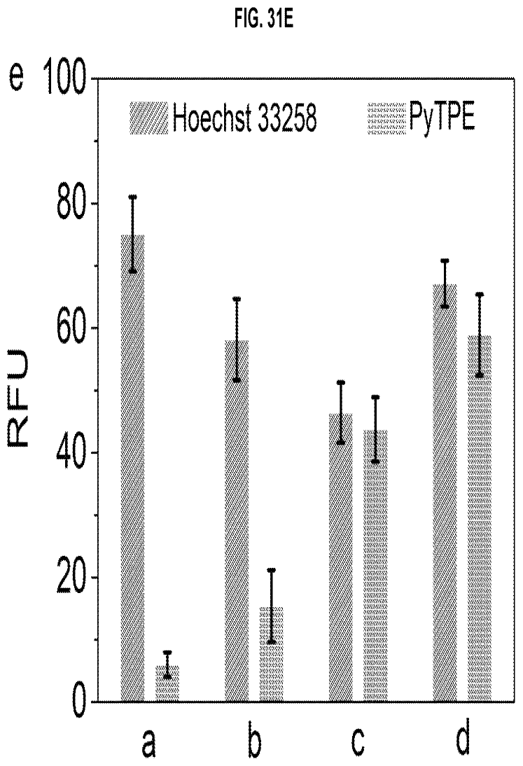

FIG. 31 illustrates CLSM images and average fluorescence intensities of Vero cells incubated with different concentration of PyTPE for 1 h, then added Hoechst 33258 for 0.5 h.

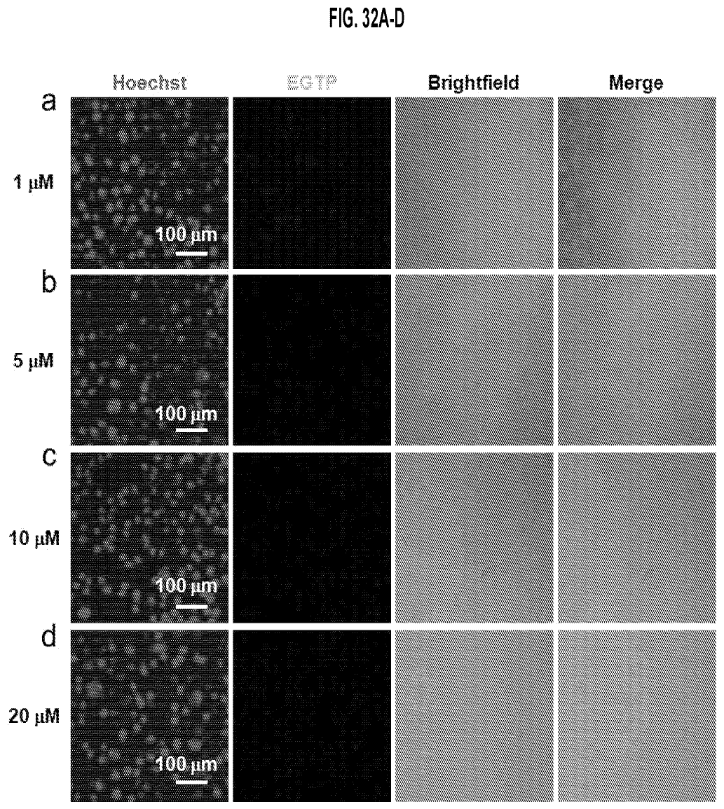

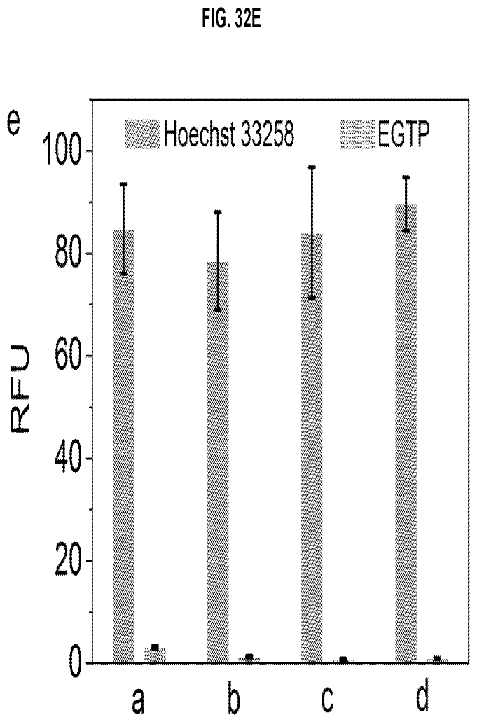

FIG. 32 illustrates CLSM images and average fluorescence intensities of Vero cells incubated with different concentration of EGTP for 1 h, then added Hoechst 33258 for 0.5 h.

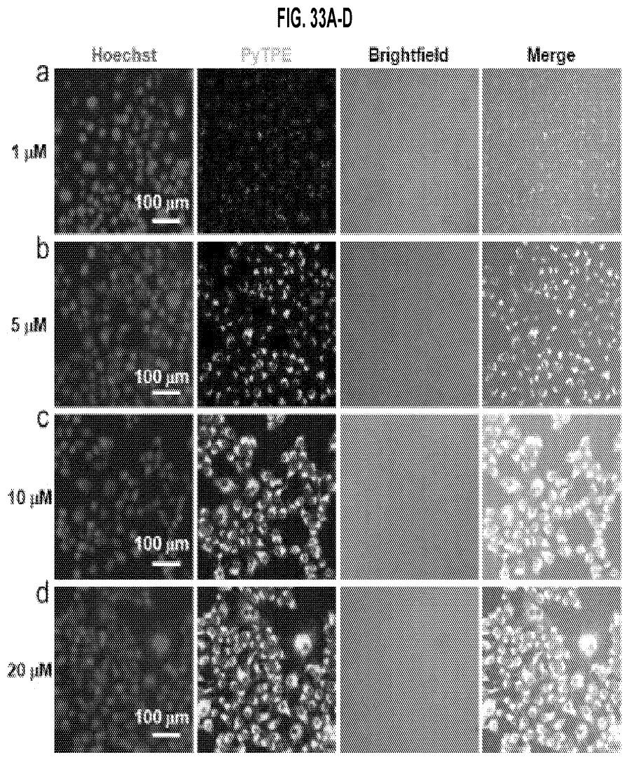

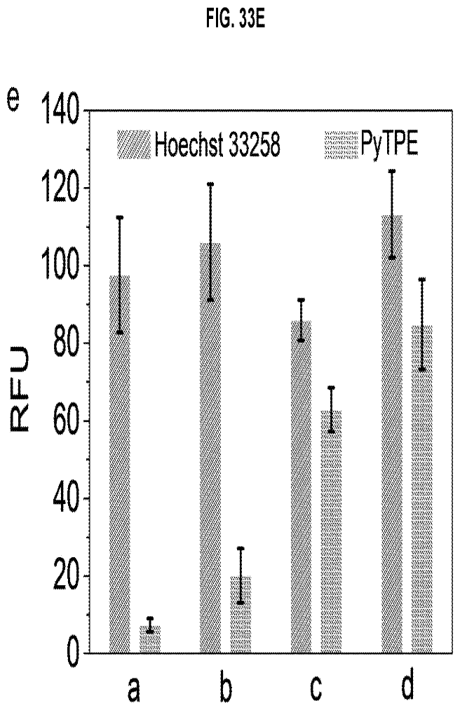

FIG. 33 illustrates CLSM images and average fluorescence intensities of TMPRSS2-Vero cells incubated with different concentration of PyTPE for 1 h, then added Hoechst 33258 for 0.5 h.

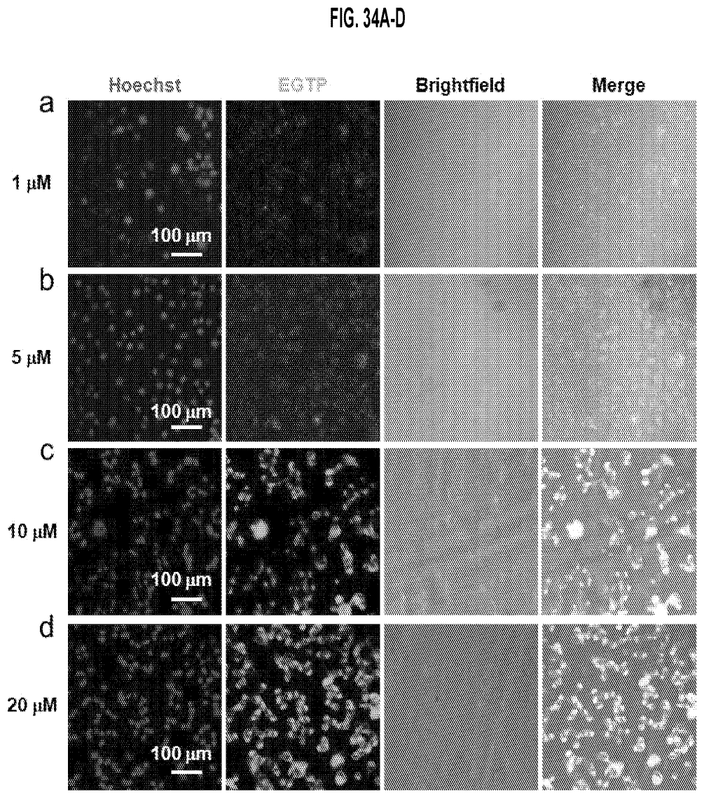

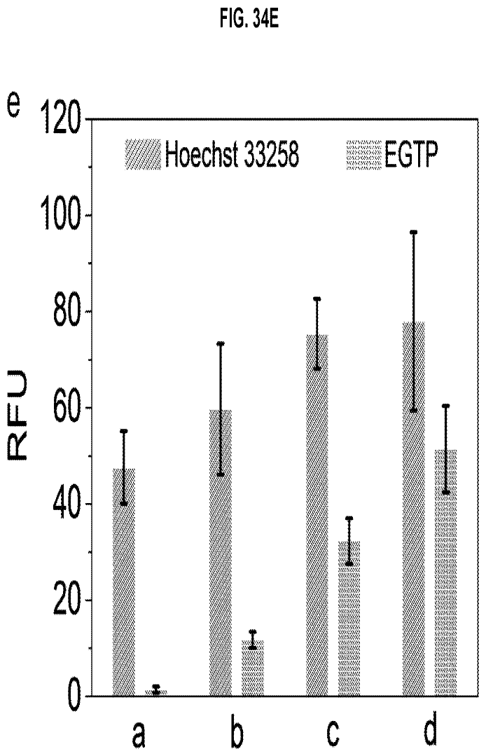

FIG. 34 illustrates CLSM images and average fluorescence intensities of TMPRSS2-Vero cells incubated with different concentration of EGTP for 1 h, then added Hoechst 33258 for 0.5 h.

FIG. 34 illustrates (a) The experimental scheme, CLSM images, and average fluorescence intensities of TMPRSS2-Vero cells incubated with (b) 100 nM GC376 and (c) 100 nM GRL0617 for 1 h, 10 μM EGTP for 1 h, then added Hoechst 33258 for 0.5 h.

### Example 3: Tunable Protease-Responsive Modular-Peptide-Conjugated AIEgens for Selective Imaging and Inhibition of SARS-CoV-2 Infected Cells

This example describes making and using synthetic peptides and polypeptides to treat or prevent viral infections.

Here, we report Mpro-responsive modular-peptide-conjugated AIEgens (PSGMR) for selective imaging and inhibition of SARS-CoV-2-infected cells. PSGMR consists of five segments (Scheme. 1a): The first is an AIEgen (PyTPE, P for short in PSGMR). PyTPE has bright yellow fluorescence, excellent biocompatibility, and good photostability.^{44, 46} Second, the self-assembling peptide (KLVFF (SEQ ID NO:5), S) is a β-sheet-forming peptide derived from β-amyloid (Aβ) protein. It can spontaneously self-assemble into amyloid fibrils through π-π stacking, hydrogen bonding, and hydrophobic interactions.47-49 Third, the spacer trimylglycine (GGG, G) can enhance flexibility and reduce steric hindrance for Mpro-substrate interactions.⁵⁰⁻⁵¹ The fourth component is the Mpro-responsive peptide (SAVLQ/SGFRKMA (SEQ ID NO:6), M),⁵²⁻⁵³ and the fifth is a positive hexamolyarginine (RRRRRR (SEQ ID NO:7), R) that increases both the solubility and cell-penetrating ability of PSGMR.⁵⁴⁻⁵⁷ These five components were covalently coupled through a Fmoc-based solid-phase peptide synthesis and a copper-catalyzed azide-alkyne click reaction. In the absence of Mpro, PSGMR is an amphiphilic molecule that is highly water-soluble with limited fluorescence. It can form loose nanoparticles due to the positive hexamolyarginine residues on the surface and hydrophobic core of PyTPE. After being cleaved by Mpro, however, the hydrophilic hexamolyarginine is separated from PSG, and the self-assembling peptides with one negative charge were exposed to the nanoparticle surface (Schemes 1b and S1). The increasing self-assembly and electrostatic attraction as well as the decreasing hydrophilicity led to PSG aggregation and nanofibers with strong yellow fluorescence. Finally, the nanofibers can selectively inhibit the growth of SARS-CoV-2-infected cells and prevent virus replication. This theranostic probe will provide an advanced avenue for selective imaging and inhibition of SARS-CoV-2 infected cells.

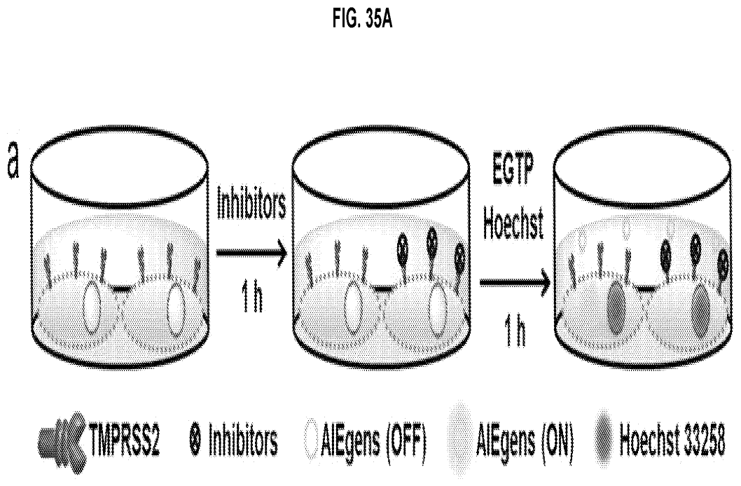

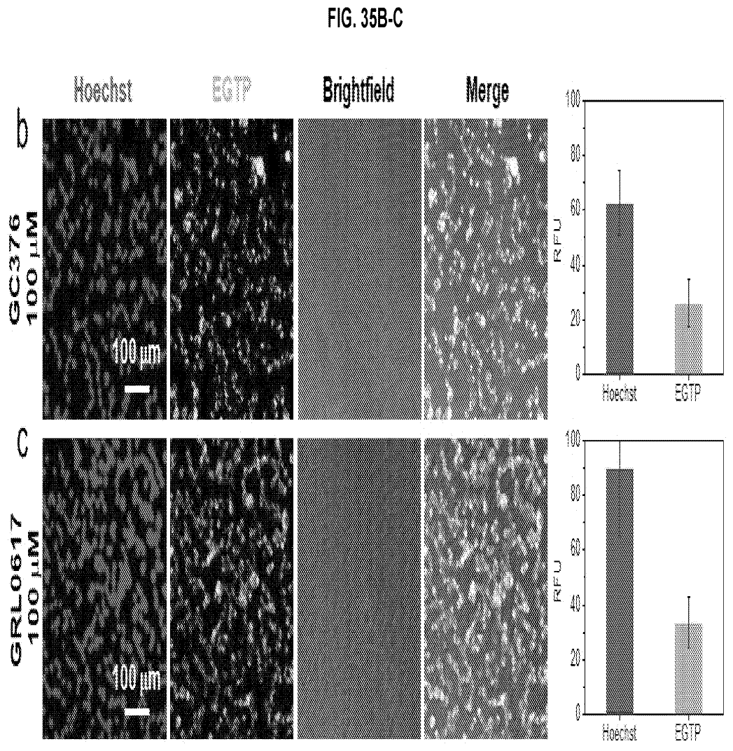

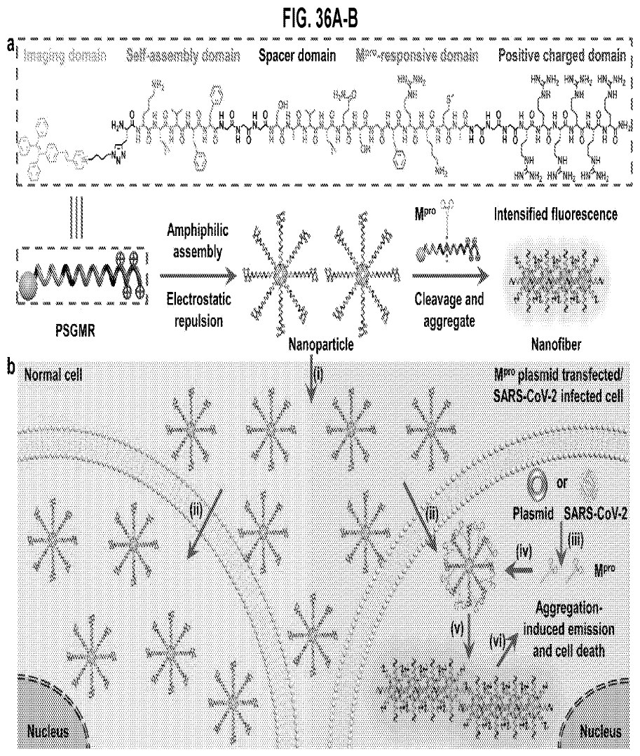

FIG. 36, or scheme 1 of Example 3, illustrates the structure and function of the exemplary PSGMR: (a) Molecular structure of PSGMR. (b) PSGMR is used for main protease (M^{pro}) detection and selective inhibition of M^{pro} plasmid transfected or SARS-CoV-2 infected cells.

### Results

Design, synthesis and characterization of PSGMR and its derivatives. PSGMR was designed based on the requirements of EISA and AIE effect via an Mpro trigger (FIG. 1a). PyTPE is a typical AIEgen used for cell imaging. KLVFF (SEQ ID NO:5) was chosen for its toxicity when aggregated into insoluble fibrils.48-49, 58-59 SAVLQ/SGFRKMA (SEQ ID NO:6) is a substrate for Mpro cleavage and can regulate the ratio of hydrophobicity and hydrophilicity and surface potential. This sequence has an area of high enzyme-digestion efficiency located in the center of PSGMR. After Mpro cleavage, SAVLQ/SGFRKMA (SEQ ID NO:6) was divided into the hydrophobic SAVLQ (SEQ ID NO:9) and hydrophilic SGFRKMA (SEQ ID NO:10). For proper arrangement of these segments, the hydrophobic PyTPE and KLVFF (SEQ ID NO:5) were located at the N-terminal while the hydrophilic hexamolyarginine part was placed at the C-terminal. Trimylglycine was added at both ends of the Mpro substrate to leave enough space for enzyme and substrate binding. All peptide domains were covalently linked via Fmoc-based solid-phase peptide synthesis. Propargylglycine (Pra) was used as a linker to couple with az-ide-functionalized PyTPE under mild conditions via a cop-per-catalyzed click reaction. Two control probes without spacer and self-assembling peptide (PSMR and PMR) were designed and synthesized to verify the Mpro accessibility and self-assembly.

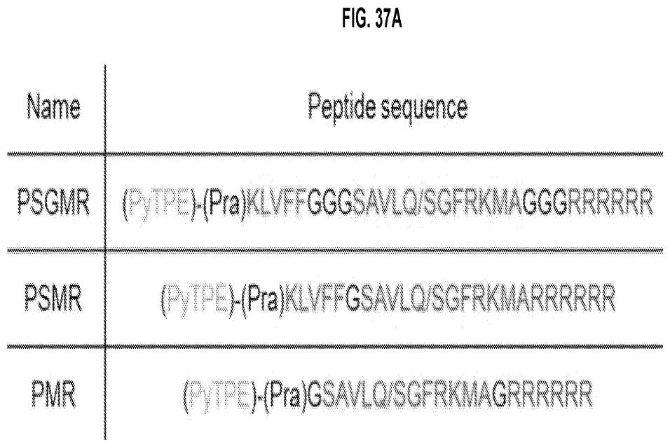

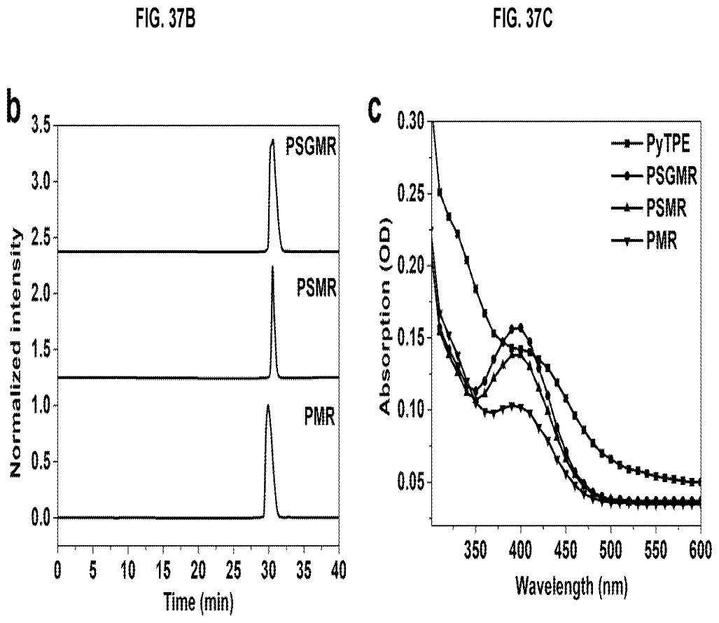

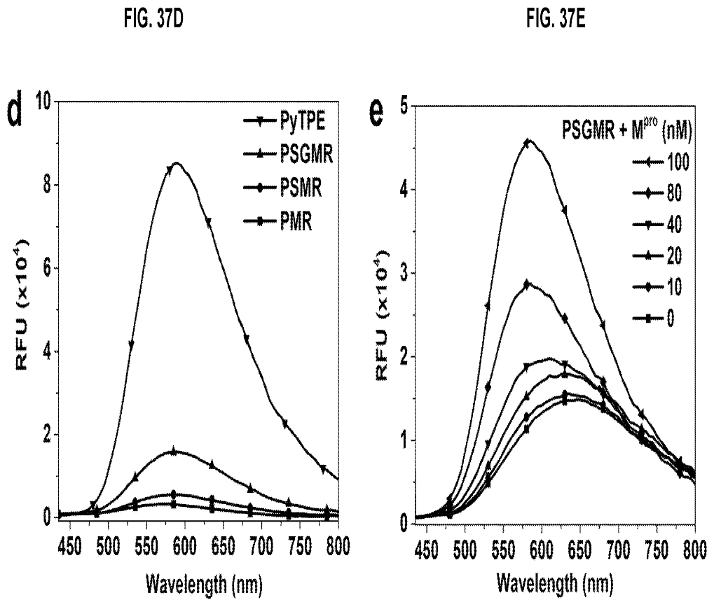

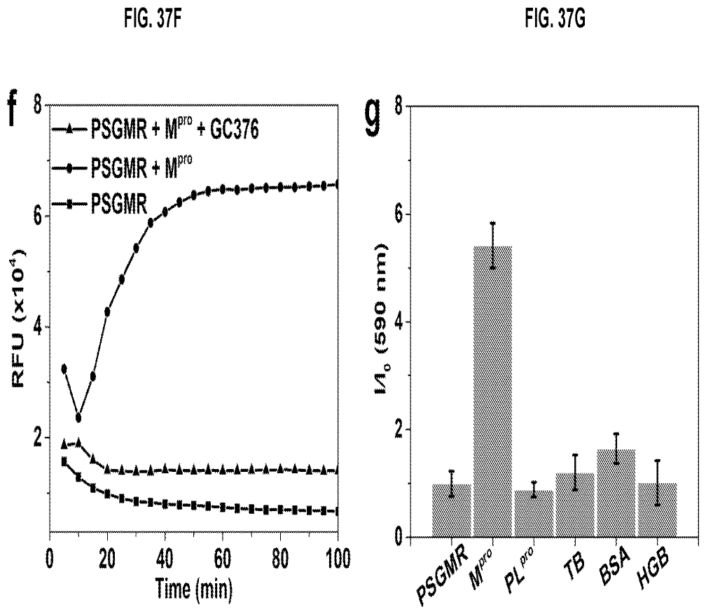

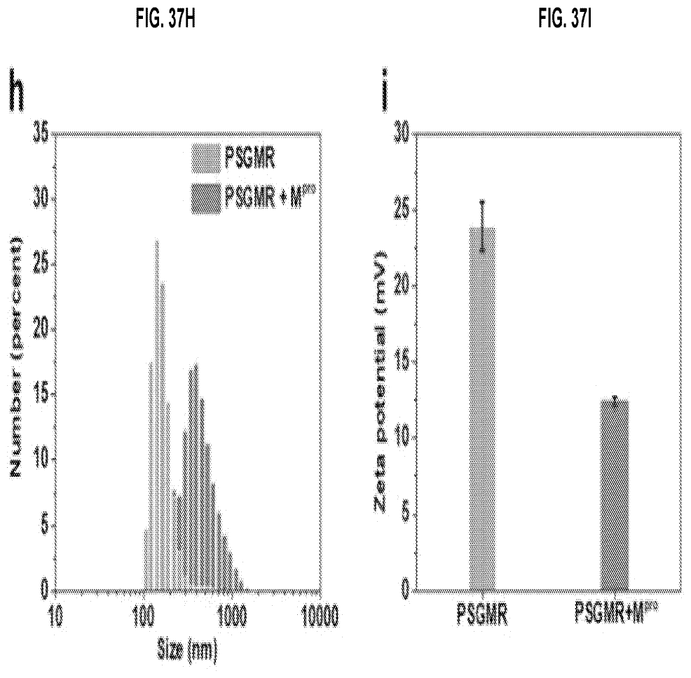

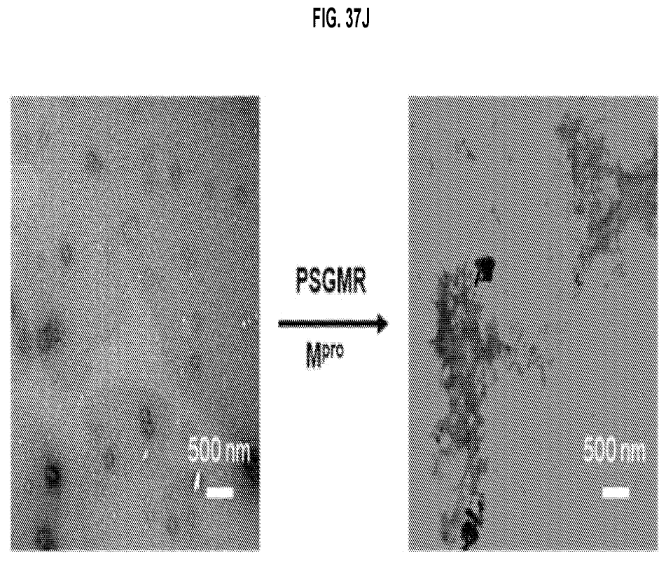

FIG. 37, or FIG. 1 of Example 3, illustrates characteristics of PSGMR and its derivatives. (a) Molecular composition, (b) high-performance liquid chromatography (HPLC), (c) absorption, and (d) fluorescence spectra of PSGMR, PSMR and PMR showed the good purity and solubility enhancement with the decreased fluorescence intensity. (e) Fluorescence spectra and (f) kinetics of PSGMR with M^{pro} and 10 mM M^{pro} GC376 at 590 nm showed the fluorescence increase because of M^{pro}. (g) Probe specificity of PSGMR with 200 nM different proteins including M^{pro}, papain-like protease (PL^{pro}), thrombin (TB), bovine serum albumin (BSA), and hemoglobin (HGB). (h) Hydrodynamic sizes, (i) zeta potential values, and (j) transmission electron microscope (TEM) images of PSGMR with M^{pro}. 10 mM probes were dissolved in Tris-HCl buffer with 1% DMSO with lₑₓ=405 nm. Scale bar=500 nm.

These products were synthesized according to previous reports with minor improvements (Schemes S2-S5, Table S1). The probes were characterized by high performance liquid chromatography (HPLC) and electrospray ionization mass spectrometry (ESI-MS) to confirm their purity (at least 95%) and chemical structures (FIG. 1b, S1-S9 in the Supporting Information). We also tested the high-resolution mass spectra (HRMS) to verify the accuracy of multiple charge peaks of PSMR in the ESI-MS (Figures S3 and S5). Figure S7 shows a strong peak at 768.2742 at-tributed to the [M+5H]5+ ion of PSGMR (calculated, 767.8326); a strong peak at 640.3345 attributed to the [M+6H]6+ ion of PSGMR (calculated, 640.0285); a strong peak at 549.0886, attributed to the [M+7H]7+ ion of PSGMR (calculated, 548.7398); and a strong peak at 480.9327 at-tributed to the [M+8H]8+ ion of PSGMR (calculated, 480.2733). The mass data of PSMR and PMR matched nicely with their calculated data. These data indicate that PSGMR, PSMR, and PMR were synthesized successfully.

Response property in solutions. We first evaluated whether these probes can be specifically cleaved by recombinant Mpro as predicted. We found that CGAVLQDDD (SEQ ID NO: 11) and AVLQFFVLKC (SEQ ID NO:12) were not cleaved by Mpro (Figure S10), but RVRRSAVLQSGFRKMAC (SEQ ID NO:13) and CGKLVFFGTSAVLQSGFRGDDD (SEQ ID NO:14) were cleaved by Mpro between Q and S (Figures S11-S12). HPLC and ESI-MS analysis also confirmed that PSGMR and PMR but not PSMR can be cleaved by Mpro between Q and S after incubation for 1 h at 37° C. in 20 mM Tri-HCl buffer (pH 8.0) (Figures S13 and S14). With 24 h of incubation, PSGMR was fully cleaved by Mpro while PSMR remained intact. These data suggest that the hydrophobic peptide or PyTPE prevent Mpro from binding to the substrate. The trimylglycine spacer between KLVFF (SEQ ID NO: 5) and the substrate improves the digestion efficiency of Mpro for this substrate. We then explored the spectral properties of PyTPE, PSGMR, PSMR, and PMR. They showed absorption spectral profiles at 350-450 nm in the Tris-HCl buffer with 1% DMSO at room temperature (FIG. 1c); 405 nm was chosen as optimal excitation wavelength. The fluorescence spectral profiles of PyTPE, PSGMR, PSMR, and PMR were 500-750 nm (FIG. 1d). The fluorescence intensity of PSGMR, PSMR, and PMR de-creased after being modified with hydrophilic peptides. The fluorescence changes of 5 μM and 10 μM PSGMR and PMR incubation were monitored upon incubation with Mpro in different media: 10 μM offered the significant fluorescence enhancement and was used for subsequent experiments (Figures S15 and S16). Particularly, the fluorescence in-tensity of PMR after incubation with Mpro was weaker than that of PSGMR because of the lack of self-assembling peptide.

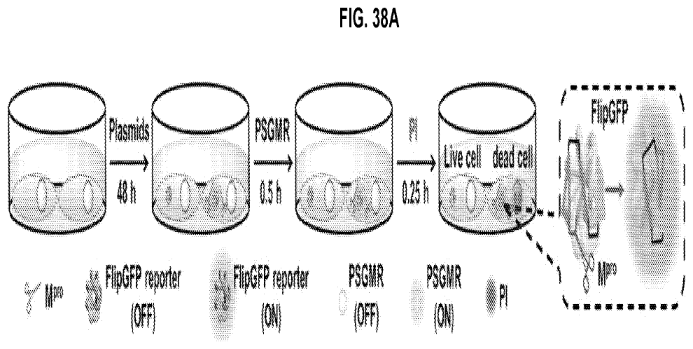

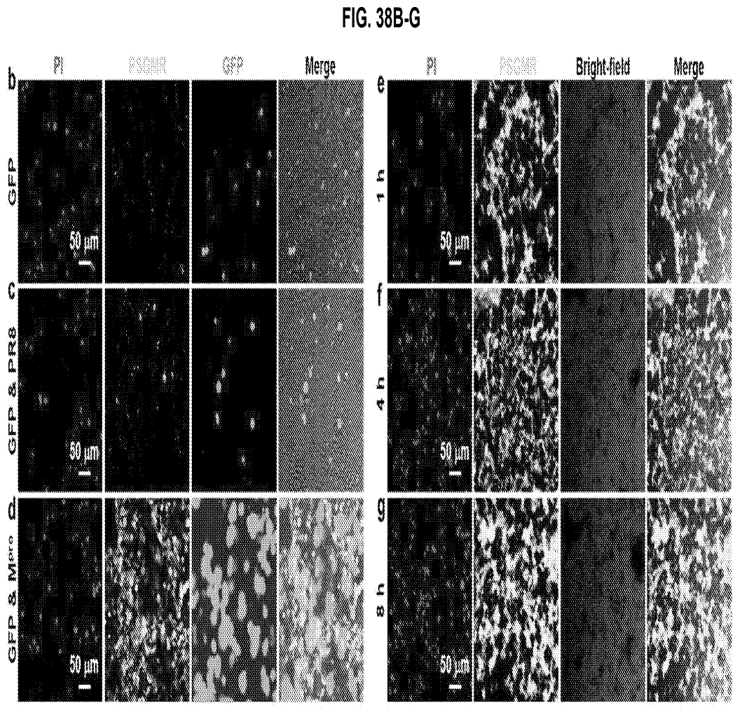

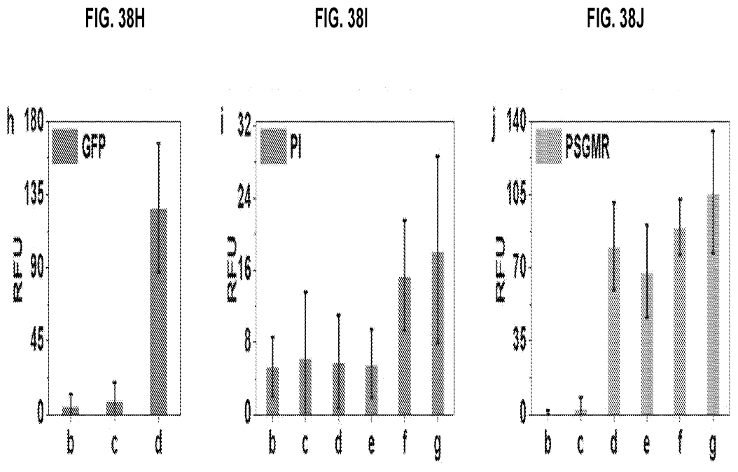

FIG. 38, or FIG. 2 of Example 3, illustrates data validating PSGMR via plasmids and reporter. (a) The experimental scheme of different plasmid transfected HEK 293T cells after incubation with PI, and PSGMR. The enlarged portion shows the imaging mechanism of FlipGFP. (b-g) Confocal laser scanning microscopy (CLSM) images of the plasmid transfected HEK 293T cells with (b, c, d) Mpro-related FLIPGFP™ reporter plasmid, (c) PR8 plasmid, (d, e, f, g) Mpro plasmid and PSGMR for different incubation time. (h-j) Average fluorescence intensities of GFP, PI and PSGMR in each panel. The FLIPGFP™ and PSGMR are respectively activated in the green and yellow channel when cells are transfected with the Mpro plasmid. The PI is activated in the red channel when cells are dead.

To validate the enzyme digestion efficiency, PSGMR was incubated with different concentrations of Mpro. The fluorescence intensity of PSGMR gradually enhanced with in-creasing Mpro concentration (FIG. 1e). Subsequent kinet-ic studies incubated PSGMR with 200 nM Mpro and 10 μM Mpro inhibitor GC375 over time.18-19 In the absence of GC376, the fluorescence intensity of PSGMR at 590 nm remarkably increased with time and plateaued within 20 min with Mpro incubation (Figure if). No increase in fluorescence was detected in the presence of the GC376, thus showing that the fluorescence increase was due to Mpro-mediated peptide cleavage. Under the same conditions, the fluorescence intensity of PSMR decreased upon incubation with Mpro (Figure S17) suggesting no cleavage of PSMR by Mpro. PSGMR was also treated under identical conditions with several commercial proteins to investigate probe specificity: papain-like protease, thrombin, bovine serum albumin, and hemoglobin. The fluorescence intensity of PSGMR was clearly enhanced selectively with Mpro (FIG. 1g). Dynamic light scattering (DLS) tests and transmission electron microscopy (TEM) of PSGMR were performed to de-termine the change of particle size and surface potential distribution after incubation with Mpro. The average hydro-dynamic size of PSGMR increased from 142 nm to 396 nm (FIG. 1h), and the mean zeta potential value decreased from 23.97 mV to 12.46 mV (FIG. 1i). These data suggest nanoparticle aggregation and a reduction of arginines on the nanoparticle surface. This aggregation was confirmed by TEM (FIG. 1j). The 20 mM Tri-HCl buffer can lead to a uniform nanoparticle (˜78 nm) (Figure S18), and β-sheet structure of PSGMR was observed after incubation with Mpro or at high concentrations (Figures S19-S22). These data illustrated that PSGMR is responsive to Mpro leading to nanoscale structural changes.

Imaging and inhibition of Mpro plasmid-transfected HEK 293T cells with PSGMR and reporter. To image Mpro in living cells, HEK 293T cells were transfected with an Mpro plasmid, influenza virus protein (PR8) plasmid, and Mpro-related FLIPGFP™ reporter plasmid to produce proteins of interest.18 The transfected cells were further incubated with PSGMR, HOECHST 33258™ (nucleus staining of all cells), and propidium iodide (PI, nucleus staining of dead cells) for confocal laser scanning microscopy (CLSM) observation. To examine the cytotoxicity and optimized concentration of probes for cell imaging, different concentrations between 1 and 40 μM of PyTPE, SGMR, PSGMR, and PMR were incubated with HEK 293T cells for 48 h under standard cell culture conditions (Figure S23). High concentration of PyTPE (20 μM) and PSGMR (40 μM) caused significant cytotoxicity. The cell viability decreased in the Mpro plasmid-transfected cells after incubation with PSGMR. According to the CLSM images of cells for 3 h incubation, blue fluorescence of HOECHST 33258™ was observed in the nucleus, and yellow fluorescence of PSGMR gradually appeared at higher concentrations, thus leading to strong background signals (Figure S24). Cells with a high concentration of PSGMR had red fluorescence from PI, thus indicating that high concentrations of PSGMR could cause significant cytotoxicity. There-fore, to avoid non-specific cleavage by proteolytic enzymes in the complex cellular microenvironment, 5 μM of probe was used for Mpro imaging for 30 min incubation; 10 μM probes for cell inhibition experiments.

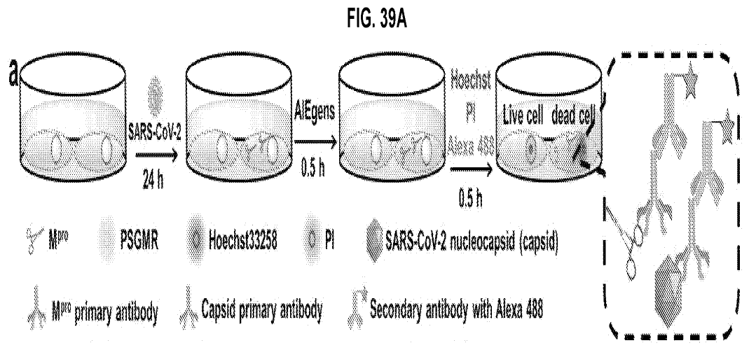

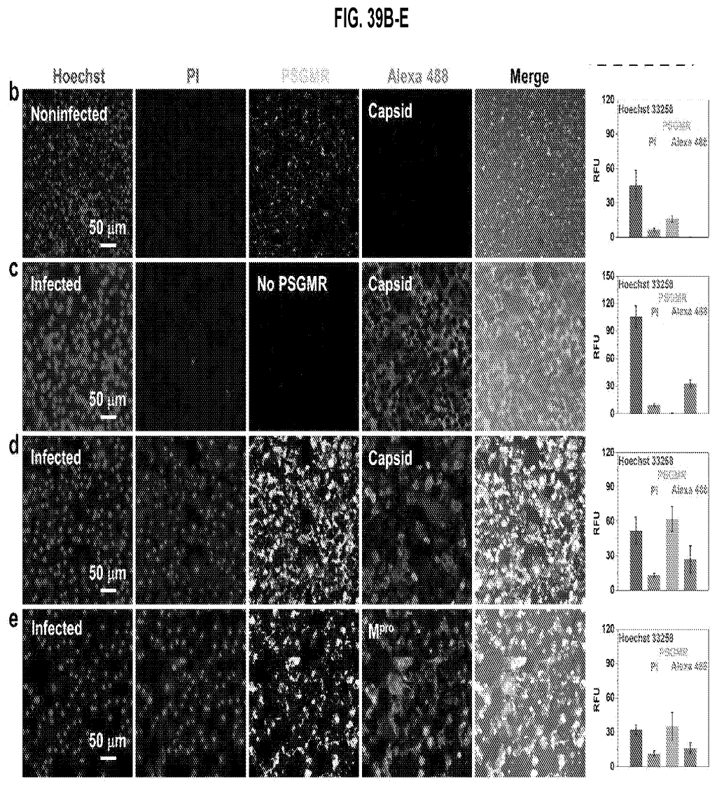

FIG. 39, or FIG. 3 or Example 3, illustrates selective imaging and inhibition of SARS-CoV-2 infected TMPRSS2-Vero cells with PSGMR. (a) The experimental scheme of SARS-CoV-2 infected TMPRSS2-Vero cells incubation with PSGMR, anti-SARS-CoV-2 nucleocapsid (Capsid) primary antibody, anti-SARS-CoV-2 Mpro primary antibody, and ALEXAFLUOR 488™-labeled secondary antibody (ALEXA 488™). The enlarged portion shows the imaging mechanism of ALEXA 488™. (b-e) CLSM images and average fluorescence intensities of (b) noninfected and (c, d, e) infected cells incubation with (b, d, e) PSGMR, (b, c, d) capsid primary antibody/ALEXA 488™, and (e) Mpro primary antibody/ALEXA 488™. The PI is activated in the red channel when cells are dead. The PSGMR and ALEXA 488™ are respectively activated in the yellow and cyan channel when cells are infected by SARS-CoV-2. Scale bar=50 μm.

To assess Mpro expression in plasmid transfected HEK 293T cells, a Mpro-related FLIPGFP™ reporter plasmid was co-transfected into Mpro plasmid or PR8 plasmid-transfected cells (FIG. 2a). The green fluorescence of the FLIPGFP™ reporter only activates after cleavage by Mpro. These plas-mid transfected cells were then incubated with 5 μM or 10 μM PSGMR, and 5 μM PI. Only weak green and yellow fluo-rescence was observed in the FLIPGFP™ reporter plasmid-transfected cells and PR8 plasmid & FlipGFP reporter plasmid co-transfected cells (FIGS. 2b and 2c). Notably, the transfection agent was toxic to cells and led to the red fluorescence.60-61 Strong green, yellow and red fluorescence was observed in the cells that were co-transfected with Mpro plasmid and FLIPGFP™ reporter plasmid (FIGS. 2d and S25). Only co-transfection of Mpro and FLIPGFP™ plasmids produced overlap between PSGMR, FLIPGFP™, and PI signal, thus indicating that Mpro was functional in these cells and more cells were dead. To validate the cell inhibitory effect of probes, 10 μM PSGMR was incubated with Mpro plasmid transfected cells for 1 h, 4 h, and 8 h (FIGS. 2e, 2f, and 2g). The green, red and yellow fluorescence intensities were markedly enhanced with the longer incubation time (FIGS. 2h, 2i, and 2j). These data proved that PSGMR can be used for selective imaging and inhibition of Mpro plasmid transfected cells based on the concentration and incubation time.

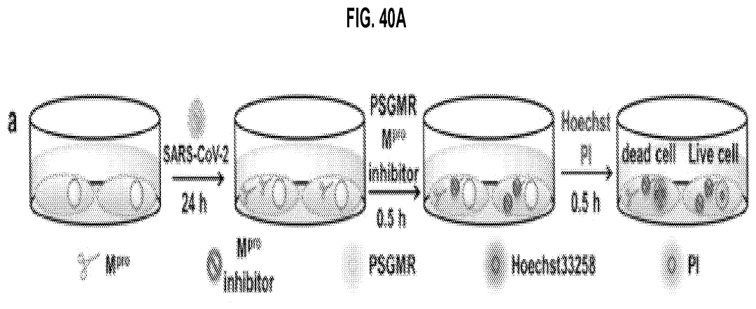

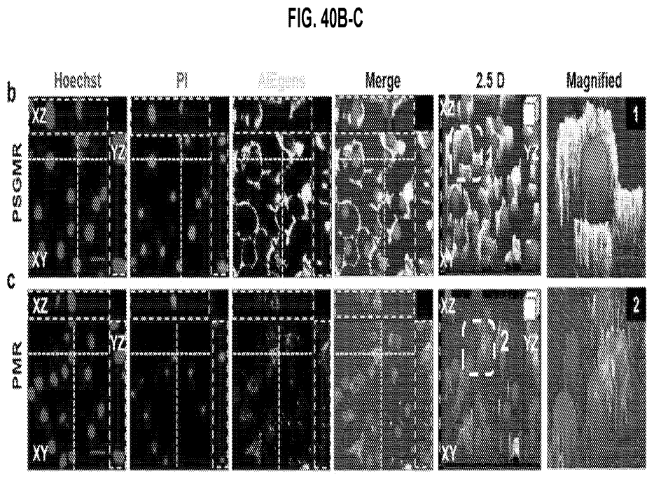

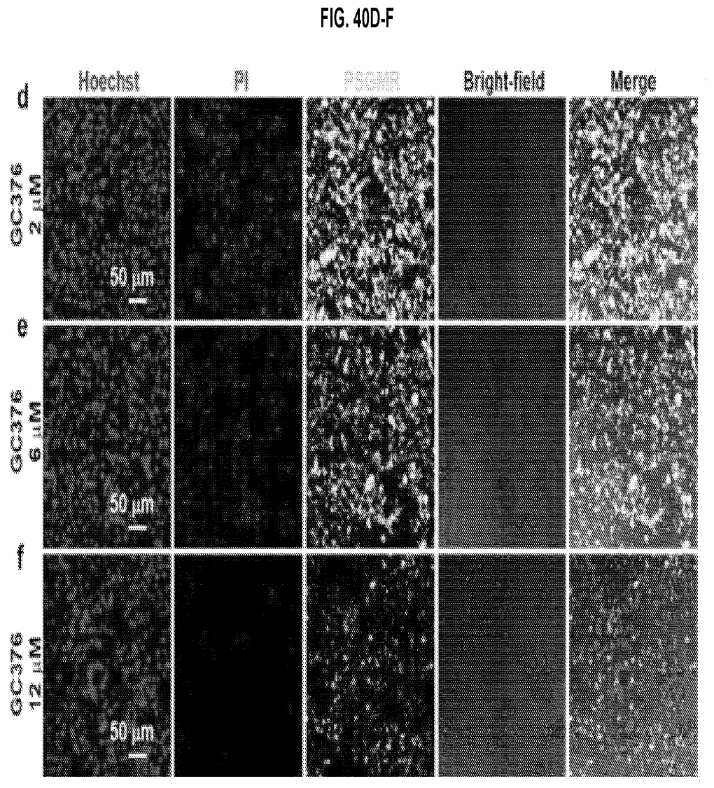

FIG. 40, or FIG. 4 of Example 3, illustrates Imaging and inhibition of SARS-CoV-2 infected TMPRSS2-Vero cells with PSGMR and Mpro inhibitor. (a) The experimental scheme of SARS-CoV-2 infected TMPRSS2-Vero cells with PSGMR and Mpro inhibitor. (b-f) CLSM images of SARS-CoV-2 infected cells with 10 μM PSGMR and 10 μM PMR. 10 μM PSGMR and 2 μM GC376, 10 μM PSGMR and 6 μM GC376, 10 μM PSGMR and 12 μM GC376. (g and h) Mean fluorescence intensity of PI and PSGMR in infected cells incubation with 10 μM PSGMR and different concentrations of GC376. In (b) and (c), the transverse and vertical dotted line denotes the XZ and YZ plane cutting line of Z-stack images, respectively. The white boxes in the 2.5 D images are magnified. Red scale bar=20 μm.

Imaging and inhibition of SARS-CoV-2 infected TMPRSS2-Vero cells with PSGMR and GC376. After vali-dating that PSGMR could selectively image and induce cyto-toxicity in Mpro plasmid transfected HEK 293T cells, we then examined whether PSGMR could similarly image and kill SARS-CoV-2-infected cells. Thus, TMPRSS2-Vero cells were infected with SARS-CoV-2 (USA-WA1/2020) at a multiplicity of infection (MOI) of 0.02 for 24 h before adding the PSGMR, Hoechst 33258, and PI. These cells were further labeled by staining viral proteins using fluorescent antibodies after cells were fixed including anti-SARS-CoV-2 nucleocapsid (Capsid) primary antibody and anti-SARS-CoV-2 Mpro primary antibody; ALEXAFLUOR 488™-labeled secondary antibody (ALEXA 488™) was used for both (FIG. 3a). At 24 h post-infection, these probes were separately incubated with the noninfected and infected cells for cell imaging. Cyan fluorescence of Alexa488 (either Capsid or Mpro) was observed in infected cells but not in uninfected cells, thus confirming that these cells were infected by SARS-CoV-2 and produced Mpro (FIGS. 3b-3e). These results were also confirmed with Western blot analysis in our previous work.19 In addition, strong yellow and red fluorescence was displayed only in infected cells treated with PSGMR rather than the uninfected cells with PSGMR or infected cells without PSGMR, thus showing that PSGMR can selectively kill SARS-CoV-2-infected cells. Upon increasing the concentration to 10 μM, the yellow and red fluorescence of the infected cells was significantly enhanced (Figure S26). Although 10 μM PMR can also inhibit infected cells, yellow and red fluorescence intensities were weaker than that of PSGMR because of the absence of a self-assembling peptide (Figure S27).

Further exploration of the intracellular distribution of probes in infected cells can help study the mechanism of action of these probes in inducing cell death. Figure S28 shows that blue and red fluorescence overlapped well in the nucleus. The yellow fluorescence of PSGMR was close to the cell membrane. Importantly, the cyan fluorescence of ALEXA 488™ for Capsid was mainly located in the cell membrane and nucleus. While the cyan fluorescence of ALEXA 488™ for Mpro was displayed in cell membrane and cytoplasm. This result clearly revealed the different locations of each protein in the infected cells. Moreover, compared with the differences between PSGMR and PMR in the noninfected and infected cells (FIG. 4a and S29), the noninfected cells with PSGMR had weak red and yellow fluorescence with normal cell morphology. The infected cells with PSGMR showed bright red and yellow fluorescence with obvious cell mor-phologic dilatation. The red and yellow fluorescence as well as cell morphology change of infected cells with PMR was less than the infected cells with PSGMR (FIGS. 4b and 4c). The average red and yellow fluorescence intensities of infected cells with PSGMR were 600% and 569% higher than the infected cells with PMR (Figure S29 and S30). These results confirmed that PSGMR was superior to PMR at selectively imaging and killing the infected TMPRSS2-Vero cells.

We then evaluated the inhibitory effect of infected cells with PSGMR and GC376. After 24 h post-infection, 10 μM PSGMR and different concentrations (2 μM, 6 μM, and 12 μM) of GC376 were incubated with the infected cells. Both red and yellow fluorescence markedly decreased with in-creasing concentrations of GC376 (FIGS. 4d-4f). Their average red and yellow fluorescence intensities decreased from 32.6 to 11.6 times and 5.8 to 3.6 times, respectively (FIGS. 4g and 4h). These data suggested that PSGMR can measure Mpro inhibition, and the cytotoxicity of PSGMR can be regulated by GC376. PSGMR and GC376 could be a com-bination therapy to prevent virus replication and track the treatment process.

### Conclusions

In summary, this work exploits the potential advantages of EISA and the AIE effect for selective detection and treatment of the virus infected cells. When combined with SARS-CoV-2 replication characteristics, a Mpro-responsive modular peptide with conjugated AIEgens named PSGMR offers selective imaging and inhibition of the Mpro plasmid transfected HEK 293T cells and SARS-CoV-2 infected TMPRSS2-Vero cells. Versus control probes without spacers and self-assembling peptides, PSGMR can be effectively cleaved by Mpro and form nanofibers with bright yellow fluorescence. Importantly, the formation process and therapeutic effect of nanofibers can be visualized by the fluorescence change and controlled by the composition of the modular-peptides, which were verified with Mpro-related FlipGFP reporter, PI, Alexa 488 staining for SARS-CoV-2 Capsid and Mpro, and Mpro inhibitor GC376. PSGMR potentially offering precise treatment of SARS-CoV-2 infection. This strategy will open new avenues for the development of theranostic agents against COVID-19 and other emerging diseases.

### Example 4: Tunable Protease-Responsive Modular-Peptide-Conjugated AIEgens for Selective Imaging and Inhibition of SARS-CoV-2 Infected Cells

This example describes making and using synthetic peptides and polypeptides to treat or prevent viral infections.

### Materials and Methods

Rink-amide resin (particle size 100 to 150 mesh; loading 0.67 mmol/g), Fmoc-protected L-amino acids, and O-benzotriazole-N, N, N′, N′-tetramethyl-uronium-hexafluorophosphate (HBTU) were purchased from AAPPTec, LLC. (Kentucky, USA). The 4-(1,2,2-triphenylvinyl) benzaldehyde, 1,4-dibromobutane, 4-methylpyrdine, HOECHST 33258™, and propidium iodide (PI) were purchased from COMBI-BLOCKS, INC.™ (San Diego, USA). Tris-(hydroxymethyl) aminomethane (Tris-base), tris-(hydroxymethyl) aminomethane hydrochlorideand (Tris-HCl), sodium azide, sodium ascorbate, copper (I) iodide, N, N-diisopropylethylamine (DIPEA), thrombin, hemoglobin, albumin bovine serum, and resazurin based in vitro toxicology assay kit were purchased from Sigma-Aldrich (Missouri, USA).

The SARS-CoV-2 Mpro expression plasmid was provided to us from Dr. Rolf Hilgenfeld, University of Lubeck, Germany. Recombinant SARS-CoV-2 Mpro was expressed and purified as described previously in 20 mM Tris-HCl buffer (pH 8.0) with 150 mM NaCl, 1 mM DTT, and 5% glycerol.1 A SARS-CoV-2 Mpro plasmid, an influenza virus protein (A/PR8/1834 NP, PR8 in short) plasmid, and a Mpro-related FLIPGFP™ reporter plasmid were a kind gift from Nicholas S. Heaton.² Mpro inhibitor GC376 was purchased from Selleckchem, LLC. (Houston, USA). HEK 293T cells were a kind gift from Dr. Liangfang Zhang. All other reagents were obtained from commercial sources and used without further purification. Deionized water (18.2 MΩ·cm) used in all experiments was purified with a MILLI-Q ACADEMIC WATER PURIFICATION SYSTEM™ (Millipore Corp., Billerica, MA, USA).

The 1H and 13C NMR spectra were measured on a BRUKER ARX 300™ NMR spectrometer using chloroform-d (CDCl3-d) or dimethyl sulfoxide-d6 (DMSO-d6) as solvent and tetramethylsilane (TMS) as internal reference. Splitting patterns are reported as s (single), d (doublet), t (triplet), and m (multiplet). Peptides were synthesized using an AAPPTec ECLIPSE™ system through standard solid phase Fmoc syntheses on rink-amide resin. Electrospray ionization mass spectrometry (ESI-MS) data was acquired by using a MICROMASS QUATTRO ULTIMA™ mass spectrometer. High resolution mass spectra (HRMS) were recorded on a BRUKER MICROTOF II™ mass spectrometer system. High performance liquid chromatography (HPLC) was performed by using a SHIMADZU™ LC-40 HPLC system under the wavelength of 195 nm, 254 nm, and 405 nm. The sample was dissolved in water or acetonitrile, applied on a ZORBAX 300 BS™ C18 column (5 um, 9.4×250 mm) from Agilent, and eluted at 1.5 mL/min with a 40 min gradient from 10% to 95% solvent B where solvent A is water (0.05% TFA solution) and solvent B is acetonitrile (0.05% TFA solution). All products were purified by HPLC to reach a purity of 90%. UV-Vis absorption and fluorescence spectra were performed on a SYNERGY H1™ microplate reader (BioTek). Confocal laser scanning microscopy images were obtained on a Zeiss LSM880 confocal laser scanning microscope (Zeiss). Dynamic light scattering (DLS) measurements were performed on a NANO-ZS90 ZETASIZER™ (Malvern) to determine hydrodynamic sizes and zeta potential values of the nanosystems studied. Transmission electron microscopy (TEM) images were acquired using an FEI TECNAI F20™ instrument at an operation voltage of 200 kV.

### Molecular Design and Imaging Mechanism.

FIG. 31, or Scheme S1 of Example 4: The mechanism of PSGMR that was used for selective imaging and inhibition of Mpro plasmid-transfected or SARS-CoV-2-infected cells. The amphipathic PSGMR with 13 positive charges can form loose nanoparticles due to the electrostatic repulsion. After cleavage by Mpro and absence of cationic polypeptides, PSG with four positive charges and C-terminal carboxyl group will decrease the electrostatic repulsion and enhance the molecular interactions. The electrostatic attraction, decreasing hydrophilicity, and increasing self-assembly all increase aggregation of PSG to form nanofibers with strong yellow fluorescence. SARS-CoV-2 Mpro sequence:³

(SEQ ID NO: 19)
SGFRKMAFPSGKVEGCMVQVTCGTTTLNGLWLDDVVYCPRHVICTSED
MLNPNYEDLLIRKSNHNFLVQAGNVQLRVIGHSMQNCVLKLKVDTANP
KTPKYKFVRIQPGQTFSVLACYNGSPSGVYQCAMRPNFTIKGSFLNGS
CGSVGFNIDYDCVSFCYMHHMELPTGVHAGTDLEGNFYGPFVDRQTAQ
AAGTDTTITVNVLAWLYAAVINGDRWFLNRFTTTLNDFNLVAMKYNYE
PLTQDHVDILGPLSAQTGIAVLDMCASLKELLQNGMNGRTILGSALLE
DEFTPFDVVRQCSGVTFQ

### Synthesis and Characterization

(1) PyTPE synthesis: 4, 5 PyTPE was synthesized according to our previous reports and the procedures in the literatures.

(2) Crude peptide synthesis: 100 mg rink amide resin was chosen in solid-phase peptide synthesis. 2×4 mL 20% piperidine in DMF, 0.1 M amino acid in 3 mL DMF (5 equivalents), 0.1 M HBTU in 3 mL DMF (5 equivalents), and 0.2 M DIPEA in 3 mL DMF (10 equivalents) were used for each coupling cycle under the protection of nitrogen. After the completion of solid phase synthesis, peptides were cleaved of the resins by treating with cleavage cocktail containing 2.5% thioanisole, 3% TIPS, 2.5% water, and 92% TFA for 3 hours followed by filtration to remove resin. Peptides were precipitated and centrifuged by addition of ice-cold diethyl ether to the filtrates. The residue was dissolved in 5 mL MeCN/H2O (v/v=1:1) solution and lyophilized. Crude peptides were purified using semi-preparative HPLC.

(3) Coupling with PyTPE: 4, 5 PyTPE (5.0 mg, 7.1 μmol), sodium ascorbate (0.7 mg, 3.6 □mol), copper (I) iodide (0.7 mg, 3.6 μmol), and the purified peptide (3.6 μmol) were dissolved in DMSO/H2O (v/v=1:1) solution, and stirred at 50° C. for 24 h under protection of nitrogen. The crude probes were purified using semi-preparative HPLC.

### Synthesis of PyTPE.

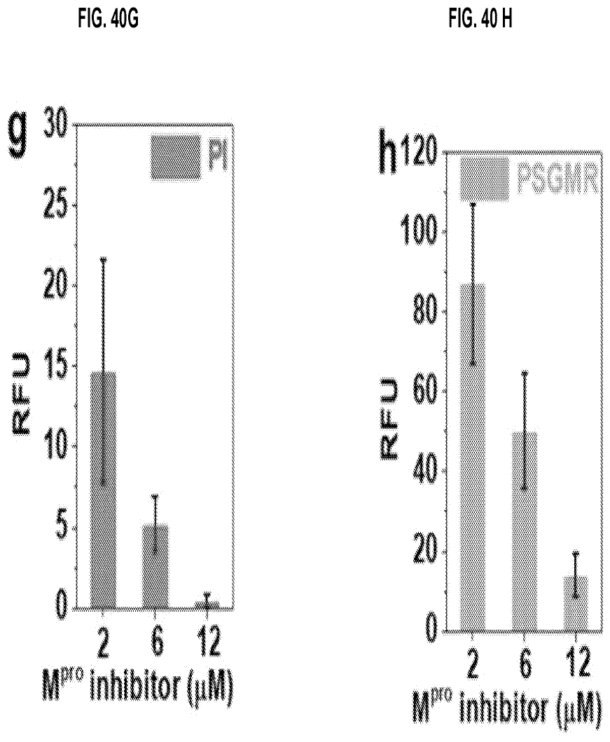

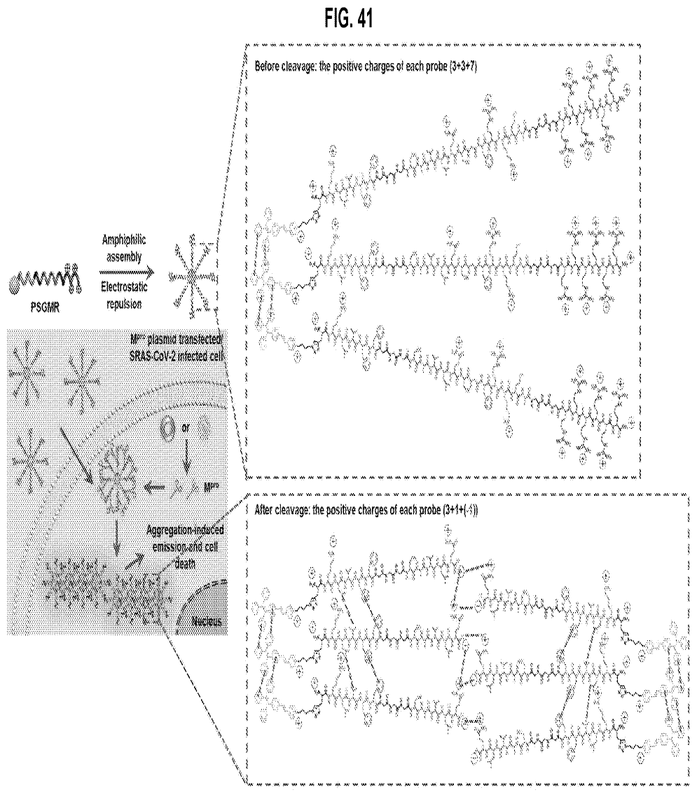

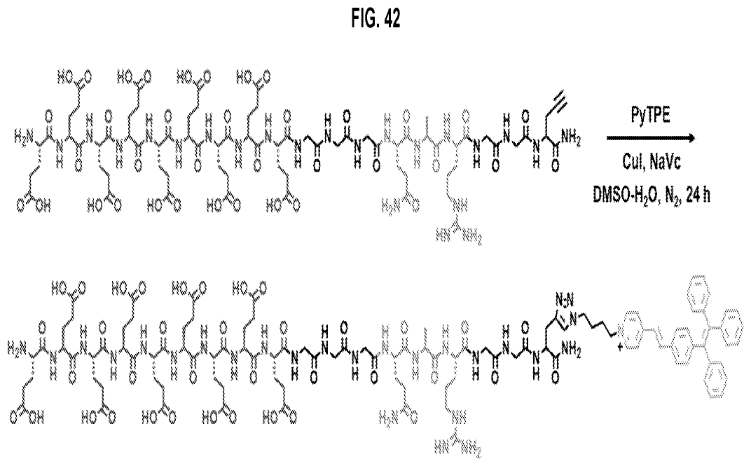

FIG. 42, or Scheme S2 of Example 4, illustrates the synthetic route of PyTPE. The main synthetic route of PyTPE is three steps: (a) Synthesis of Py-I. (b) Synthesis of Py-N3, (c) Synthesis of PyTPE, HRMS (ESI) m/z: [M]+ calcd. for 533.2700; found, 533.3771.

### Synthesis of PSMR.

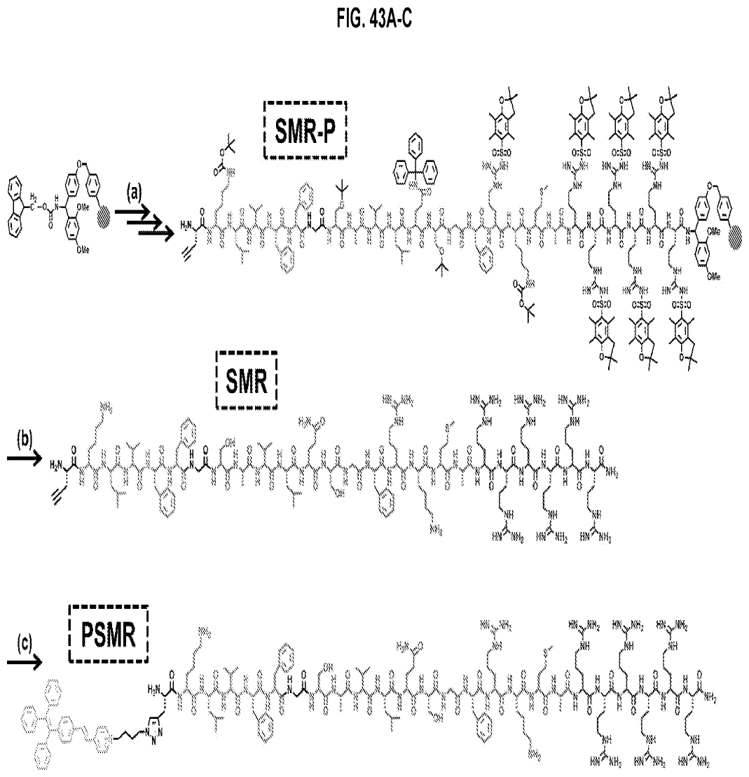

FIG. 43, or Scheme S3, of Example 4, illustrates the synthetic route of PSMR.

The main synthetic route of PSMR is three steps through standard solid phase Fmoc synthesis and copper-catalyzed azide-alkyne click reaction: (a) Synthesis of SMM-P. (b) Synthesis of SMR, HRMS (ESI) m/z: [M+4H]4+/4 calcd. for 754.9458; found, 755.5761. [M+5H]5+/5 calcd. for 604.1582; found, 604.6490. [M+6H]6+/6 calcd. for 503.6331; found, 504.0028. (c) Synthesis of PSMR, HRMS (ESI) m/z: [M+4H]4+/4 calcd. for 888.2633; found, 888.6069. [M+5H]5+/5 calcd. for 710.8122; found, 711.2593. [M+6H]6+/6 calcd. for 592.5115; found, 592.8325. [M+7H]7+/7 calcd. for 508.0109; found, 508.3657.

### Synthesis of PSGMR.

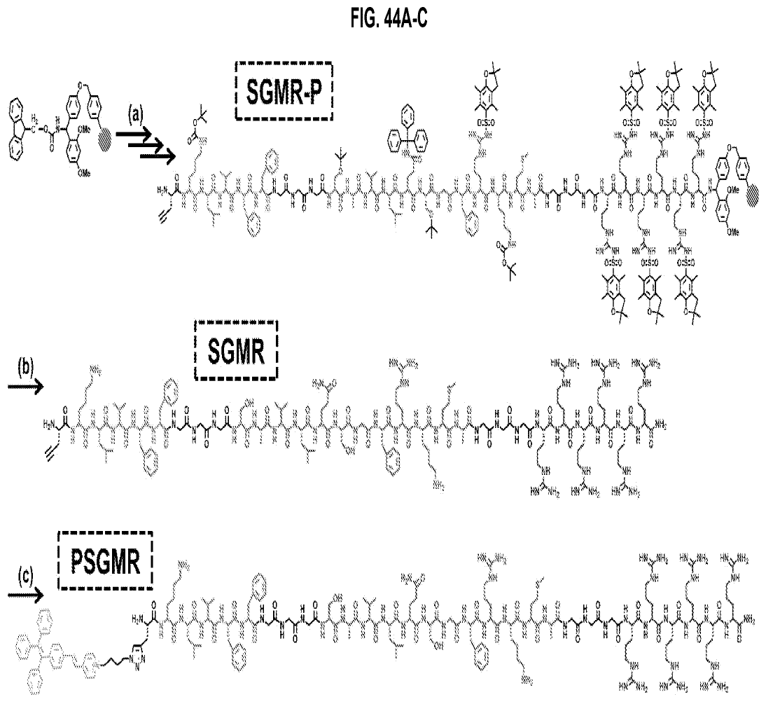

FIG. 44, or Scheme S4 of Example 4, illustrates the synthetic route of PSGMR.

The main synthetic route of PSGMR is three steps through standard solid phase Fmoc synthesis and copper-catalyzed azide-alkyne click reaction. (a) Synthesis of SGMR-P. (b) Synthesis of SGMR, HRMS (ESI) m/z: [M+4H]4+/4 calcd. for 826.2213; found, 826.8641. [M+5H]5+/5 calcd. for 661.1786; found, 661.6305. [M+6H]6+/6 calcd. for 551.1502; found, 551.5397. (c) Synthesis of PSGMR, HRMS (ESI) m/z: [M+5H]5+/5 calcd. for 767.8326; found, 768.2742. [M+6H]6+/6 calcd. for 640.0285; found, 640.3345. [M+7H]7+/7 calcd. for 548.7398; found, 549.0886. [M+8H]8+/8 calcd. for 480.2733; found, 480.9323.

### Synthesis of PMR.

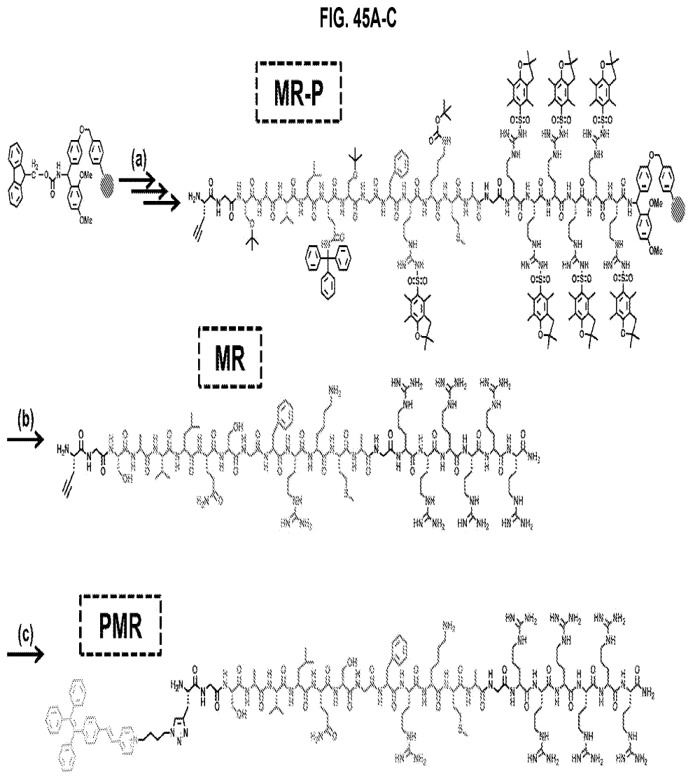

FIG. 45, or Scheme S5 of Example 4, illustrates the synthetic route of PMR.

The main synthetic route of PMR is three steps through standard solid phase Fmoc synthesis, thiol-maleimide Michael addition and copper-catalyzed azide-alkyne click reaction. (a) Synthesis of MR-P. (b) Synthesis of MR, HRMS (ESI) m/z: [M+3H]3+/3 calcd. for 813.8029; found, 814.3572. [M+4H]4+/4 calcd. for 610.5042; found, 611.1230. [M+5H]5+/5 calcd. for 488.6849; found, 489.0517. (c) Synthesis of PMR, HRMS (ESI) m/z: [M+4H]4+/4 calcd. for 743.9216; found, 744.3828. [M+5H]5+/5 calcd. for 595.3388; found, 595.7583. [M+6H]6+/6 calcd. for 496.2837; found, 496.5610. [M+7H]7+/7 calcd. for 425.5299; found, 425.8732.

Enzymatic assay with PSGMR, PSMR, and PMR. The stock solution of PyTPE, PSGMR, PSMR, and PMR in 20 mM Tris-HCl buffer (pH=8.0) was diluted with Mpro assay buffer (20 mM Tris-HCl buffer (pH 8.0) with 150 mM NaCl, 1 mM DTT, and 5% glycerol) to make 5 and 10 μM working solutions. Recombinant Mpro was added into the working solution and then diluted to a total of 100 μl or 200 μl with deionized water. The reaction mixture was incubated at 37° C. for 1 h for UV-Vis absorption and photoluminescence (PL) measurement. The solution was excited at 405 nm, and the emission was collected from 430 nm to 800 nm.

Cell culture and plasmid transfection. HEK 293T cells were cultured in Dulbecco's Modified Eagle Medium (DMEM) with 10% fetal calf serum (FBS) and 1% penicillin streptomycin (PS, 10000 IU penicillin and 1000 μg/mL streptomycin, multicell) in a cell culture plates at 37° C. in a humidified atmosphere containing 5% CO2. For plasmid transfection, cells were treated with 100 μl to 1000 mL poly-L-lysine for 20 min before being seeded with HEK 293T cells. After 24 h incubation, OPTI-MEM™ plasmids (1 μg/μl to 3 μg/μl), and TRANSIT-LT™ (Mirus) were successively mixed and incubated at room temperature for 15 min before being added to cells dropwise according to the manufacturer's instructions.

Incubation living cells with probes. For confocal laser scanning microscopy imaging, HEK 293T cells or the plasmid transfected HEK 293T cells were seeded into cell culture dishes at a density of 2.0×10⁵ in growth medium (DMEM supplemented with 10% FBS, 200 mL). After overnight incubation, the cells were washed with phosphate-buffered saline (PBS, pH 7.4) three times. A solution of the indicated probe in medium or PBS was then added, and the cells were incubated in a 5% CO₂ atmosphere at 37° C. for further use. HOECHST 33258™, PI, and ALEXAFLUOR™ 488 were subsequently added for probe incubation. The supernatant was then discarded, and the cells were washed gently twice with PBS and fixed with 2% paraformaldehyde (PFA) at room temperature for 20 min prior to optical imaging.

Viral infection. SARS-CoV-2 isolate WA1 (USA-WA1/2020, BEI NR-52281) was passaged once through primary human bronchial epithelial cells differentiated at air-liquid interface to select against Furin site mutations. Virus was then expanded by one passage through TMPRSS2-Vero cells. Supernatants were clarified and stored at −80° C., and titers were determined by fluorescent assay on TMPRSS2-Vero cells. TMPRSS2-Vero cells were infected with a multiplicity of infection (MOI) of 0.02 FFU per cell 24 h before incubation with probes. The SARS-CoV-2 noninfected and infected TMPRSS2-Vero cells were washed with Dulbecco's phosphate-buffered saline (DPBS) to remove FBS-containing media, then incubated with 5 or 10 μM probes for 30 min and fixed with 4% formaldehyde for 30 min. Cells were then stained using the nucleocapsid antibody or SARS-CoV-2 Mpro antibody and 5 μM Hoechst 33258. All work with SARS-CoV-2 was conducted in Biosafety Level-3 conditions at the University of California San Diego.

Immunofluorescence. Fixed cells were washed with PBS and then with PBS including 1% BSA and 0.1% TritonX-100. Cells were incubated with primary antibody against SARS-CoV-2 nucleocapsid protein (1:2000, GENETEX GTX135357™) or Mpro (1:100, CELL SIGNALING TECHNOLOGY #51661™) in PBS including 1% BSA and 0.1% TRITON X-100™ overnight at 4° C. Cells were washed and incubated with ALEXA 488™ goat anti-rabbit secondary antibody (Thermo Fisher Scientific) in PBS including 1% BSA for 1 h at room temperature followed by 3×PBS washes.

Confocal laser scanning microscopy. The fluorescence signals of cells were detected using a ZEISS LSM880™ confocal laser scanning microscope (Zeiss), equipped with a 63/1.42 numerical aperture oil-immersion objective lens. A 405-nm laser was chosen for the excitation of Hoechst 33258, the emission was collected at 420-460 nm. A 405 nm laser was chosen for the excitation of AIEgens and the emission was collected at 550-620 nm. A 488 nm laser was chosen for the excitation of GFP, the emission was collected at 500-530 nm. A 488 nm laser was chosen for the excitation of PI and the emission was collected at 595-650 nm. A 488 nm laser was chosen for the excitation of ALEXAFLUOR 488™, the emission was collected at 500-550 nm. All fluorescence images were analyzed with Zeiss Image software (Zeiss).

Cytotoxicity assay. The cytotoxic potential of PyTPE, SGMR, PSGMR, and PMR were assessed using the HEK 293T cells for 48 h incubation in quadruplicate in a 96-well plate. The fluorescence of Resazurin solution at 690 nm using an excitation wavelength of 560 nm was recorded by a SYNERGY H1™ microplate reader (BioTek) with standard operation procedures.

TABLE S1
sequences used in this study.

| Peptide name | Sequences |
|---|---|
| 1 | CGAVLQ/DDD |
| | (SEQ ID NO: 11) |
| 2 | AVLQ/FFVLKC |
| | (SEQ ID NO: 12) |
| 3 | RVRRSAVLQ/SGFRKMAC |
| | (SEQ ID NO: 13) |
| 4 | CGKLVFFGTSAVLQ/SGFRGDDD |
| | (SEQ ID NO: 14) |
| MR | (Pra)GSAVLQ/SGFRKMAGRRRRRR |
| | (SEQ ID NO: 16) |
| SMR | (Pra)KLVFFGSAVLQ/SGFRKMARRRRRR |
| | (SEQ ID NO: 17) |
| SGMR | (Pra)KLVFFGGGSAVLQ/SGFRKMAGGGRRRRRR |
| | (SEQ ID NO: 18) |

FIG. 46, or Figure S10 of Example 4, illustrates HPLC results of different peptides before and after incubation with Mpro showing that some AVLQ (SEQ ID NO: 20) peptide sequence cannot be cleaved by Mpro. (a) CGAVLQDDD (SEQ ID NO: 11) was not cleaved by Mpro. (b) AVLQFFVLKC (SEQ ID NO:12) was not cleaved by Mpro. (c) RVRRSAVLQSGFRKMAC (SEQ ID NO:13) was cleaved by Mpro. (d) CGKLVFFGTSAVLQSGFRGDDD (SEQ ID NO:14) was cleaved by Mpro.

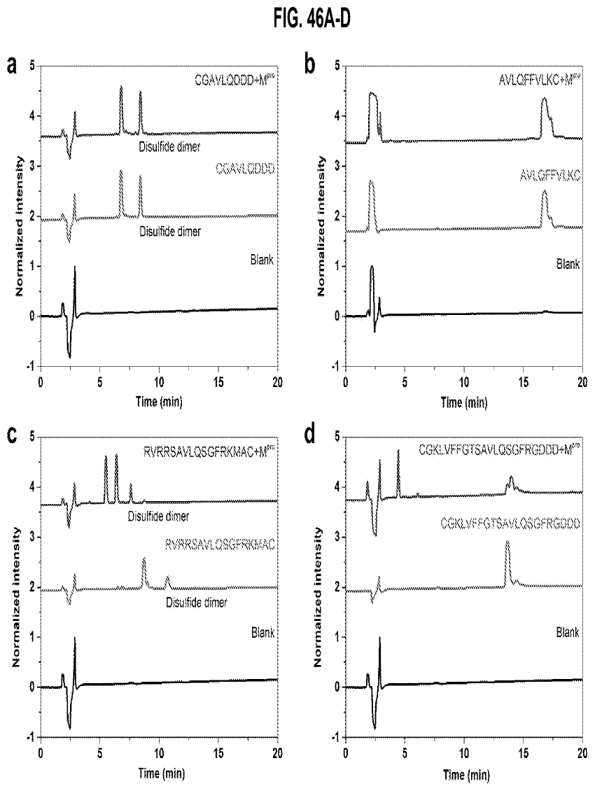

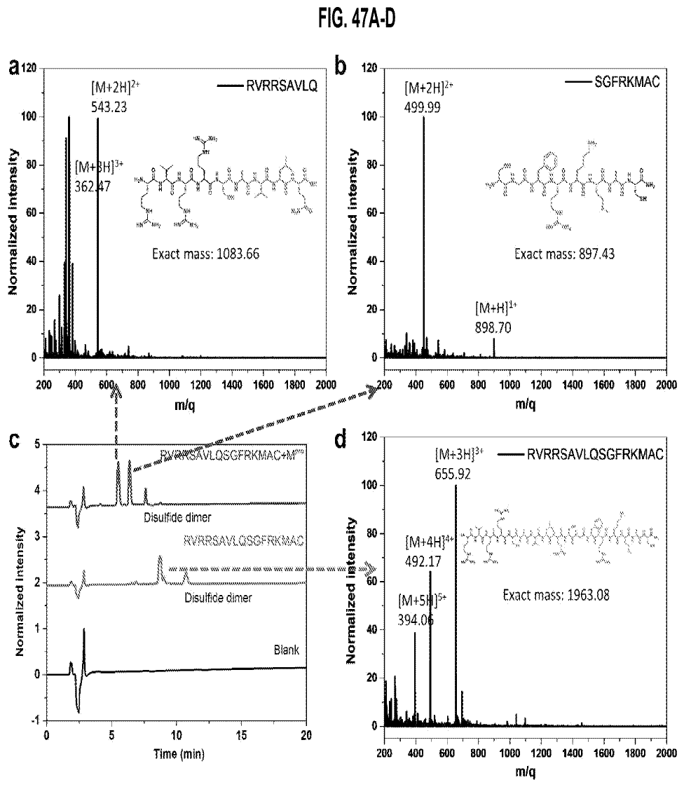

FIG. 47, or Figure S11 of Example 4, illustrates HPLC and ESI-MS results of compound RVRRSAVLQSGFRKMAC (SEQ ID NO: 13) before and after incubation with M^{pro} showing that RVRRSAVLQSGFRKMAC (SEQ ID NO:13) can be cleaved by M^{pro} after AVLQ (SEQ ID NO: 20). (a) ESI-MS results of RVRRSAVLQ (SEQ ID NO: 21). (b) ESI-MS results of SGFRKMAC. (c) HPLC results of RVRRSAVLQSGFRKMAC (SEQ ID NO:13) before and after incubation with M^{pro}. (d) ESI-MS results of RVRRSAVLQSGFRKMAC (SEQ ID NO:13).

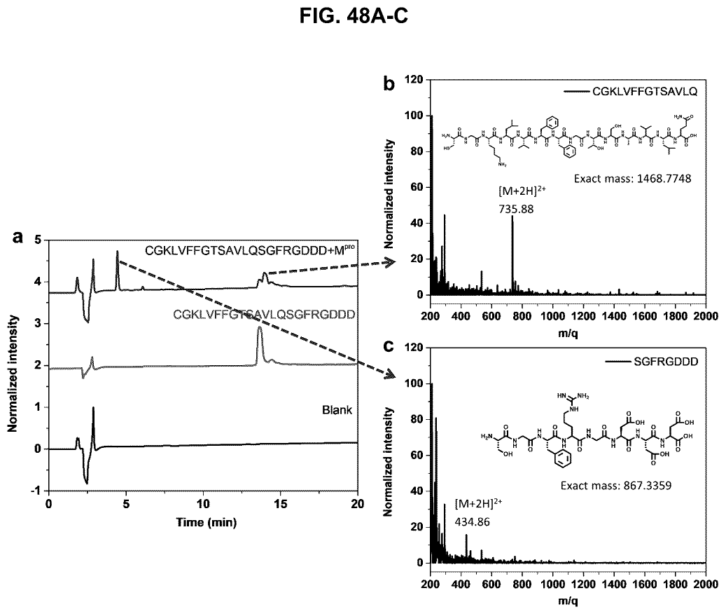

FIG. 48, or Figure S12 of Example 4, illustrates HPLC and ESI-MS results of compound CGKLVFFGTSAVLQSGFRGDDD (SEQ ID NO:14) before and after incubation with M^{pro} showing that CGKLVFFGTSAVLQSGFRGDDD (SEQ ID NO:14) can be cleaved by M^{pro} after AVLQ (SEQ ID NO: 20). (a) HPLC results of CGKLVFFGTSAVLQSGFRGDDD (SEQ ID NO:14) before and after incubation with M^{pro}. (b) ESI-MS results of CGKLVFFGTSAVLQ (SEQ ID NO:8). (c) ESI-MS results of SGFRGDDD (SEQ ID NO:15).

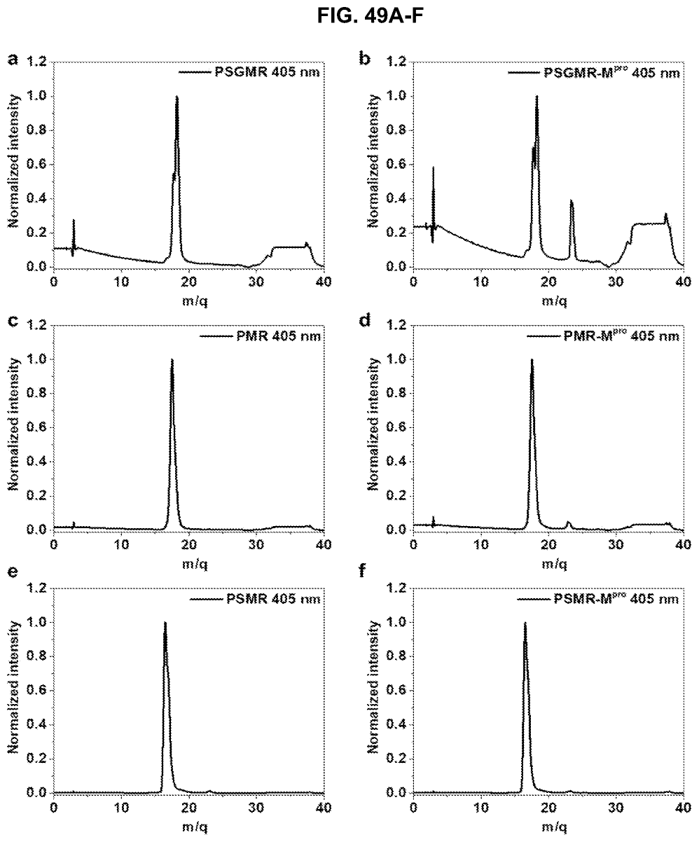

FIG. 49, or Figure S13 of Example 4, illustrates HPLC results of compounds PSGMR, PMR, and PSMR before and after incubation with M^{pro} showing that PSGMR and PMR not PSMR can be cleaved by M^{pro}. (a-b) PSGMR. (c-d) PMR. (e-f) PSMR.

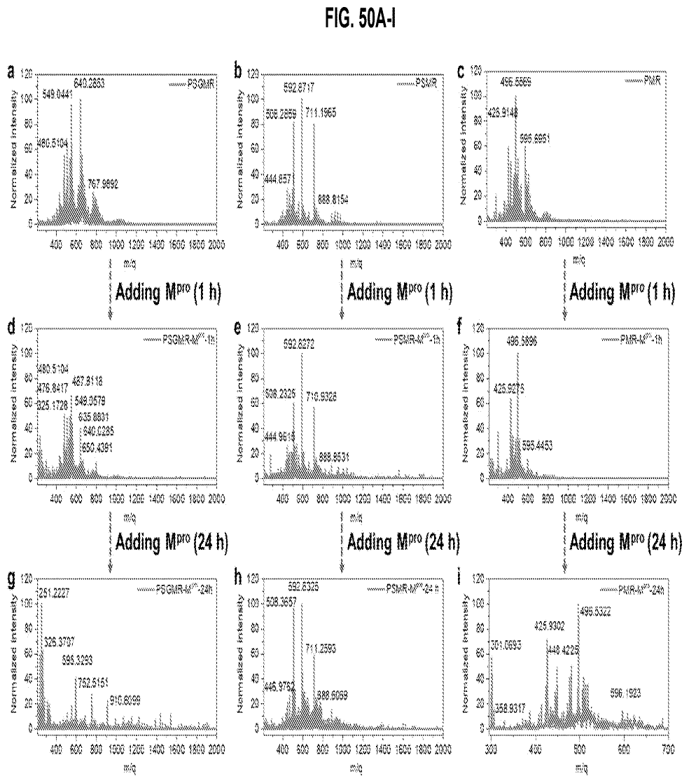

FIG. 50, or Figure S14 of Example 4, illustrates ESI-MS results of compounds PSGMR, PSMR, and PMR before and after incubation with M^{pro} for 1 h and 24 h showing that PSGMR and PMR but not PSMR can be cleaved by M^{pro}. (a, d, g) PSGMR. (b, e, h) PSMR. (c, f, i) PMR.

FIG. 51, or Figure S15 of Example 4, illustrates time-dependent fluorescence spectra of (a, c) 5 μM and (b, d) 10 μM PSGMR incubation with M^{pro} in 20 mM Tris-HCl buffer and DMEM showing that high concentrations of PSGMR can aggregate easily with strong fluorescence both in 20 mM Tris-HCl buffer and DMEM.

FIG. 52, or Figure S16 of Example 4, illustrates time-dependent fluorescence spectra of (a, c) 5 μM and (b, d) 10 μM PMR incubation with M^{pro} in 20 mM Tris-HCl buffer and DMEM showing that high concentrations of PMR can easily aggregate with strong fluorescence both in 20 mM Tris-HCl buffer and DMEM.

FIG. 53, or Figure S17 of Example 4, illustrates time-dependent fluorescence spectra of 10 μM PSMR incubation with M^{pro} in 20 mM Tris-HCl buffer showing that PSMR cannot aggregate with weak fluorescence in 20 mM Tris-HCl buffer.

FIG. 54, or Figure S18 of Example 4, illustrates (a) transmission electron microscope (TEM) images and (b) hydrodynamic size of 20 mM Tris-HCl buffer.

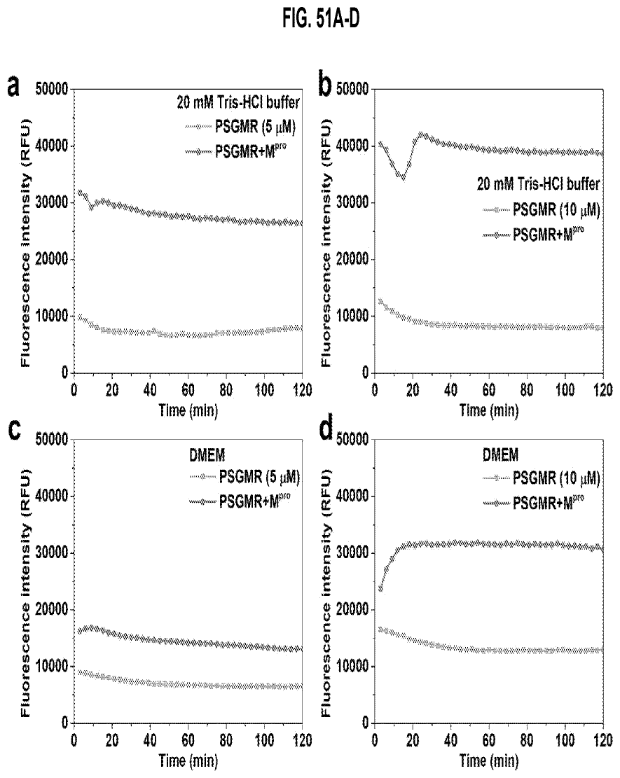

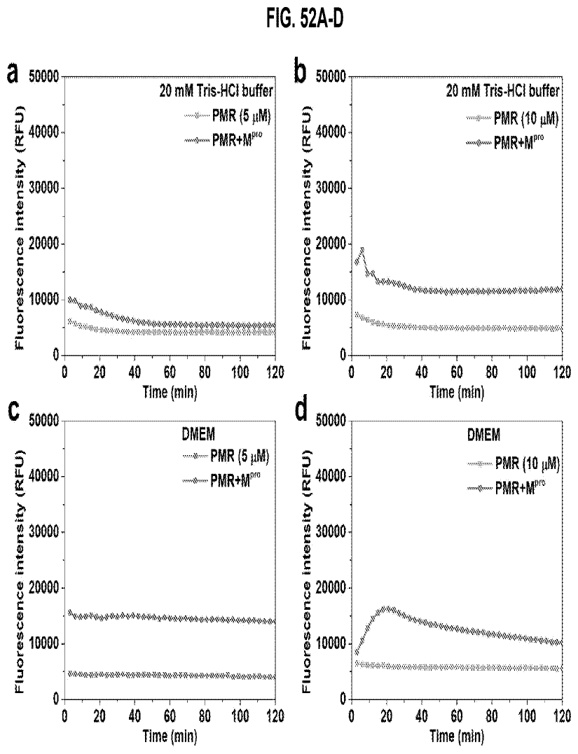

FIG. 55, or Figure S19 of Example 4, illustrates (a) Transmission electron microscope (TEM) images and (b) hydrodynamic sizes of 1 μM PSGMR incubated with and without M^{pro} in 20 mM Tris-HCl buffer.

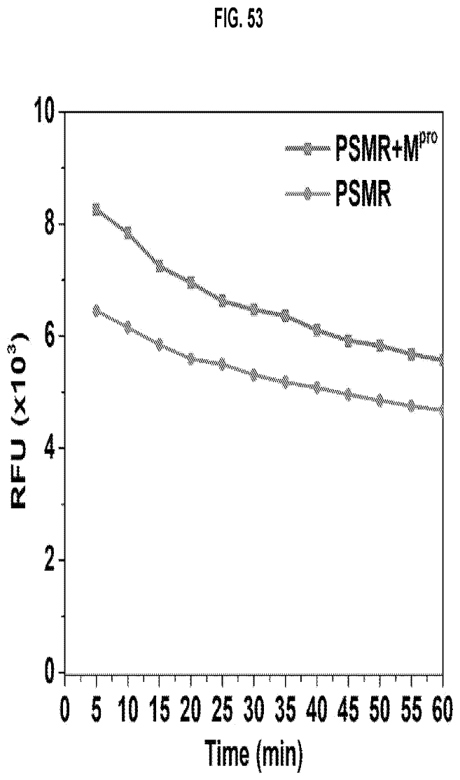

FIG. 56, or Figure S20 of Example 4, illustrates (a) Transmission electron microscope (TEM) images and (b) hydrodynamic sizes of 10 μM PSGMR incubated with and without M^{pro} in 20 mM Tris-HCl buffer.

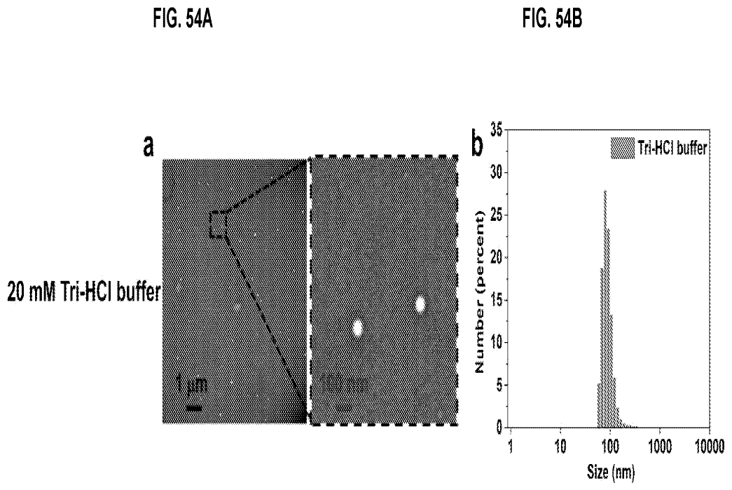

FIG. 57, or Figure S21 of Example 4, illustrates (a) Transmission electron microscope (TEM) images and (b) hydrodynamic sizes of 100 μM PSGMR incubated with and without M^{pro} in 20 mM Tris-HCl buffer.

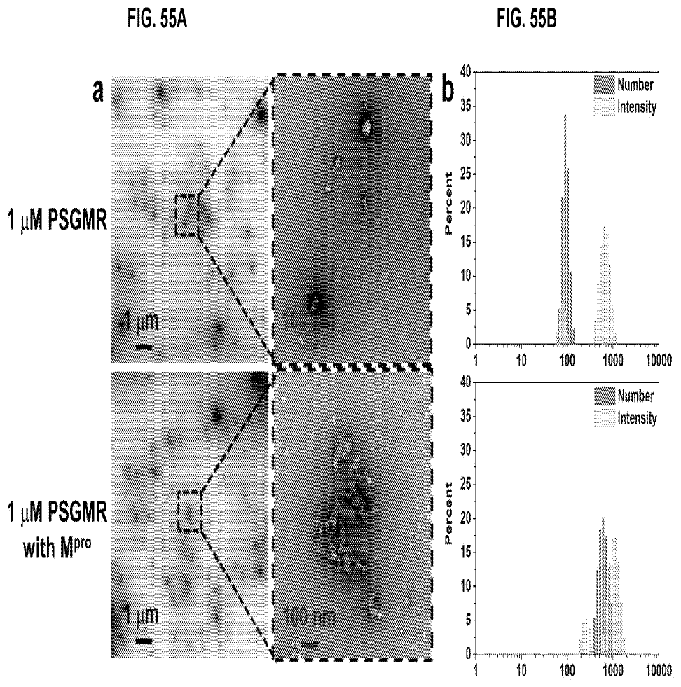

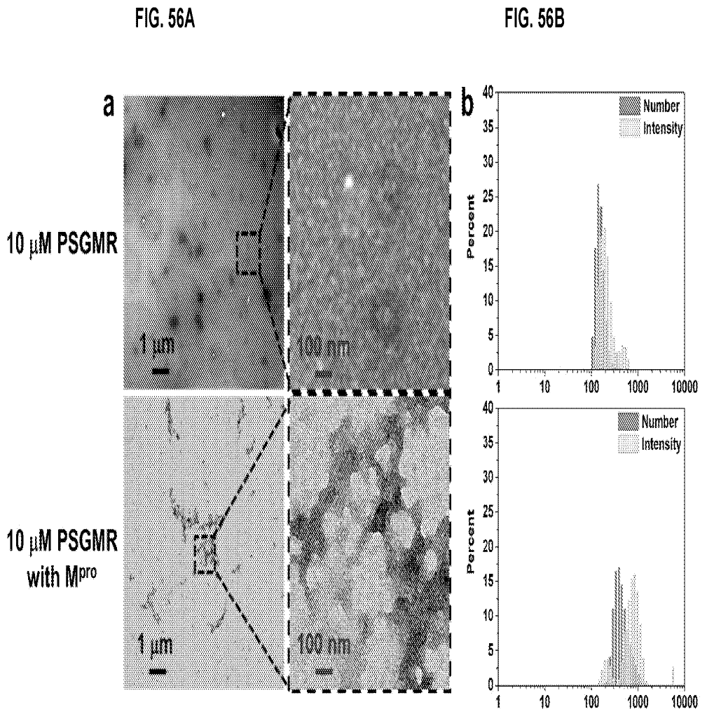

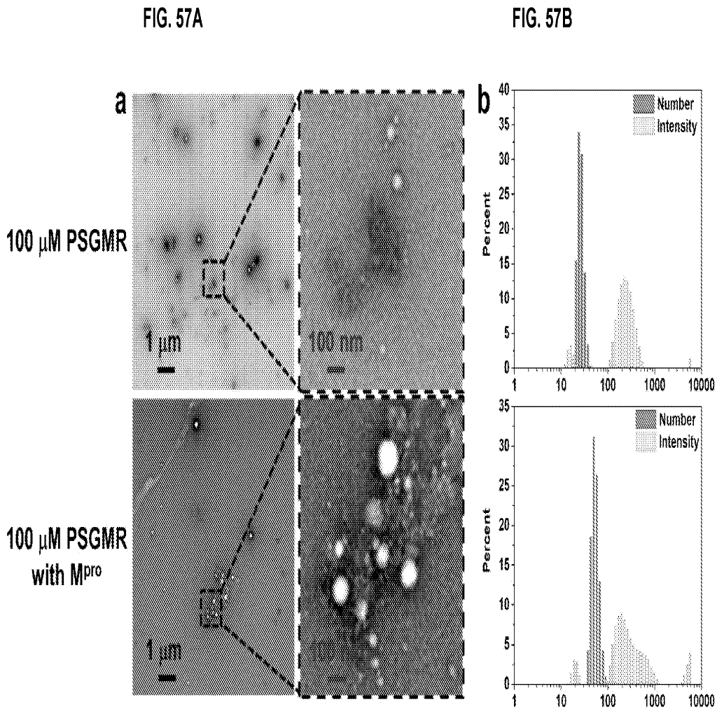

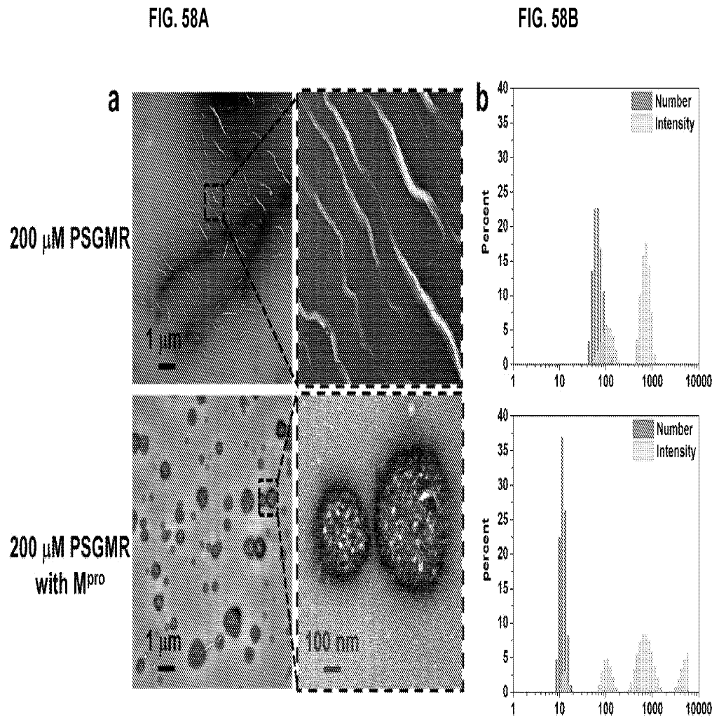

FIG. 58, or Figure S22 of Example 4, illustrates (a) Transmission electron microscope (TEM) images and (b) hydrodynamic sizes of 200 μM PSGMR incubated with and without M^{pro} in 20 mM Tris-HCl buffer.

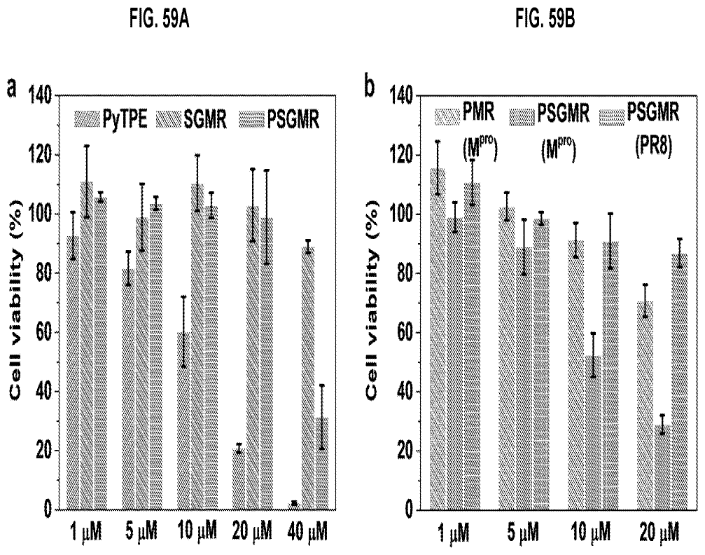

FIG. 59, or Figure S23 of Example 4, illustrates Metabolic activity of HEK 293T cells and plasmid transfected HEK 293T cells were studied with (a) (1 μM, 5 μM, 10 μM, 20 μM, and 40 μM) PyTPE, (1 μM, 5 μM, 10 μM, 20 μM, and 40 μM) SGMR, (1 μM, 5 μM, 10 μM, 20 μM, and 40 μM) PSGMR; (b) (1 μM, 5 μM, 10 μM, and 20 μM) PMR (M^{pro} plasmid), (1 μM, 5 μM, 10 μM, and 20 μM) PSGMR (M^{pro} plasmid), and (1 μM, 5 μM, 10 μM, and 20 μM) PSGMR (PR8 plasmid) for 48 h incubation using a fluorescence resazurin assay. The results show that the higher concentration of 10 μM of PyTPE and PSGMR (M^{pro} plasmid) are obviously cytotoxic.

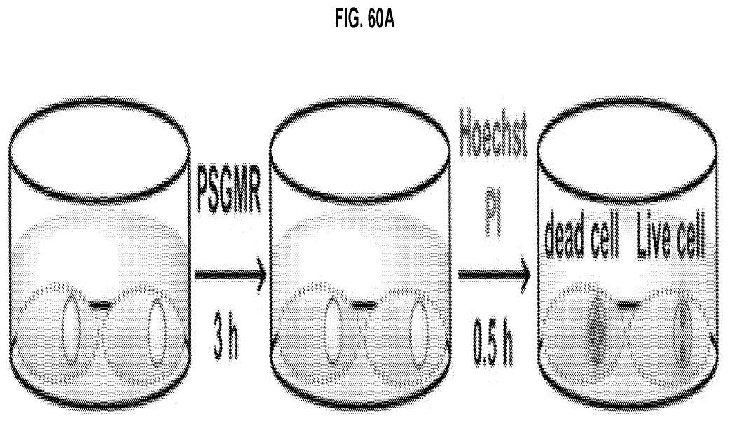

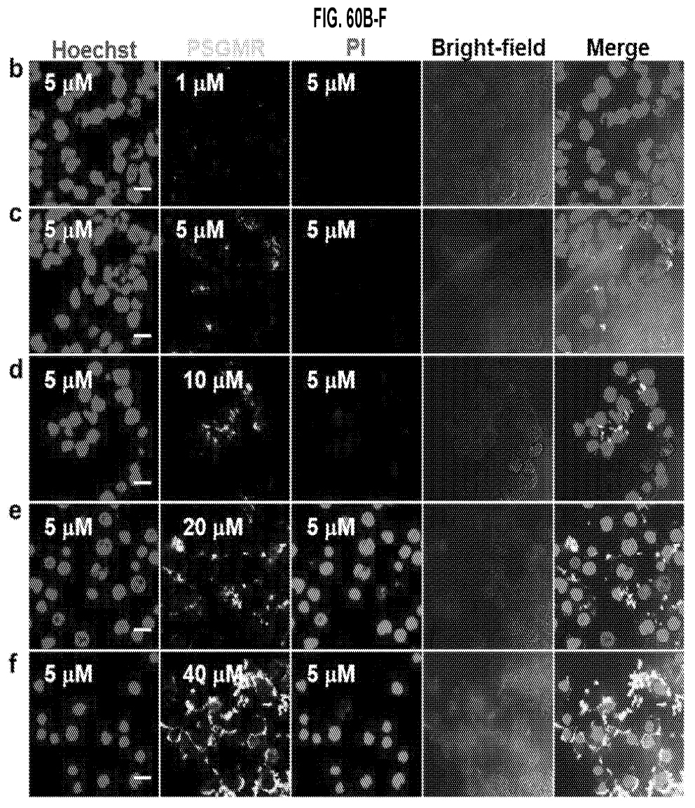

FIG. 60, or Figure S24 of Example 4, illustrates Confocal laser scanning microscopy (CLSM) images of HEK 293T cells incubated with Hoechst 33258, PSGMR, and PI. (a) The experimental scheme. Cell images of HEK 293T cells incubated with (b-f) 1 μM, 5 μM, 10 μM, 20 μM, and 40 μM PSGMR, 5 μM HOECHST 33258™, and 5 μM PI for 3 h, respectively. (g) The average fluorescence intensities of HEK 293T cells incubation with HOECHST 33258™, PSGMR, and PI. Scale bar=20 μm.

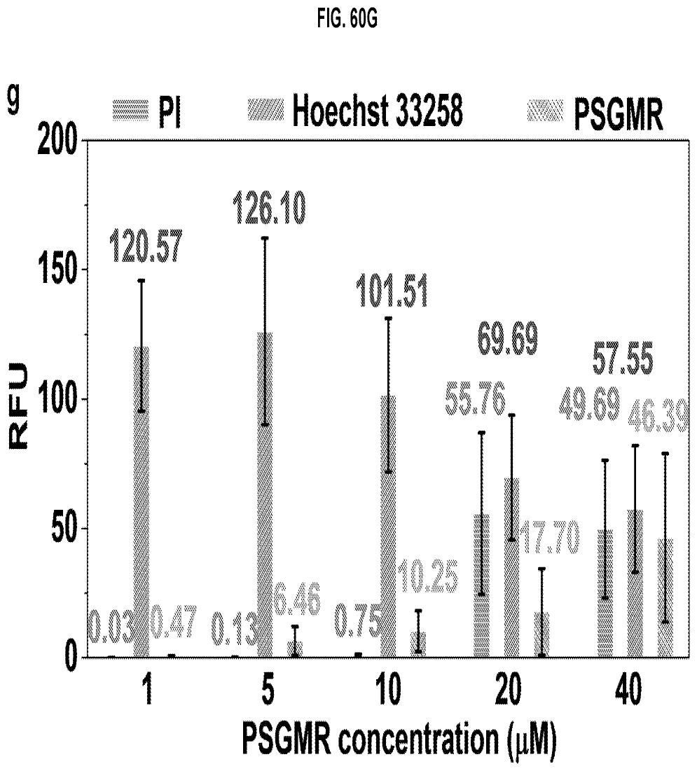

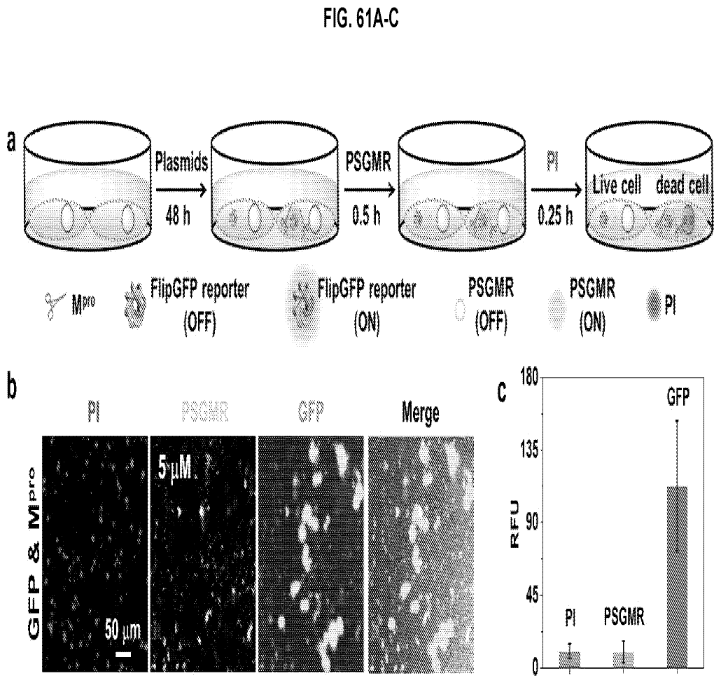

FIG. 61, or Figure S25 of Example 4, illustrates CLSM images and average fluorescence intensities of M^{pro}-related FLIPGFP™ reporter plasmid, and M^{pro} plasmid transfected HEK 293T cells incubated with PSGMR, and PI, showing that the red, yellow and green fluorescence turn-on in these cells. (a) The experimental scheme. (b) CLSM images. (c) Average fluorescence intensities of PI, PSGMR and GFP in each panel. Scale bar=50 μm.

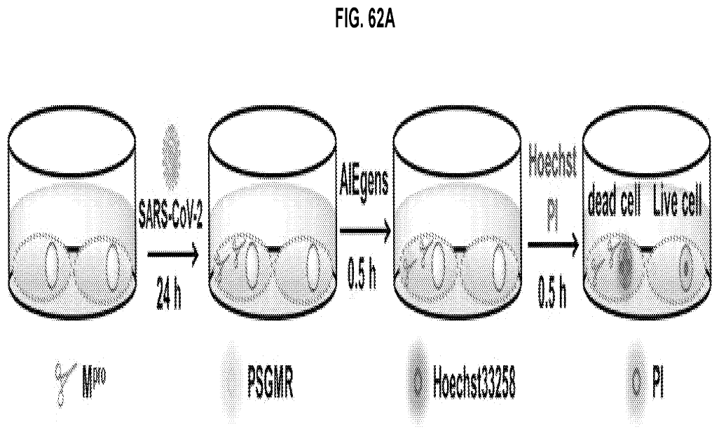

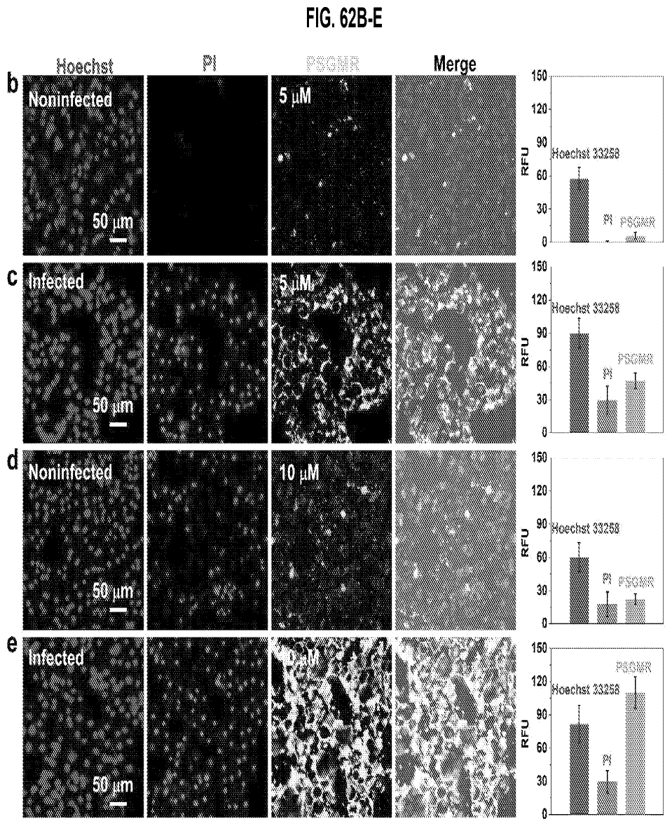

FIG. 62, or Figure S26 of Example 4, illustrates CLSM images and average fluorescence intensities of SARS-CoV-2 infected TMPRSS2-Vero cells incubation with Hoechst 33258, PI, and PSGMR. (a) The experimental scheme. Cell images and average fluorescence intensities of (b, d) noninfected TMPRSS2-Vero cells incubation with 5 μM and 10 μM PSGMR, and (c, e) SARS-CoV-2 infected TMPRSS2-Vero cells incubation with 5 μM and 10 μM PSGMR. Scale bar=50 μm.

FIG. 63, or Figure S27 of Example 4, illustrates CLSM images and average fluorescence intensities of SARS-CoV-2 infected TMPRSS2-Vero cells incubation with Hoechst 33258, PI, and PMR. (a) The experimental scheme. Cell images and average fluorescence intensities of (b, d) noninfected TMPRSS2-Vero cells incubation with 5 μM and 10 μM PMR, and (c, e) SARS-CoV-2 infected TMPRSS2-Vero cells incubation with 5 μM and 10 μM PMR. Scale bar=50 μm.

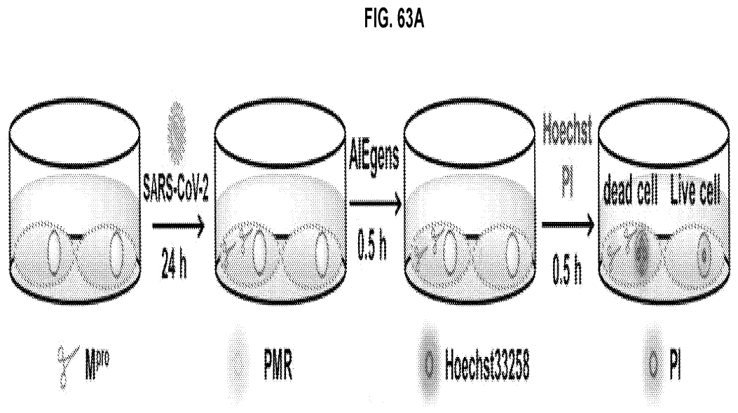

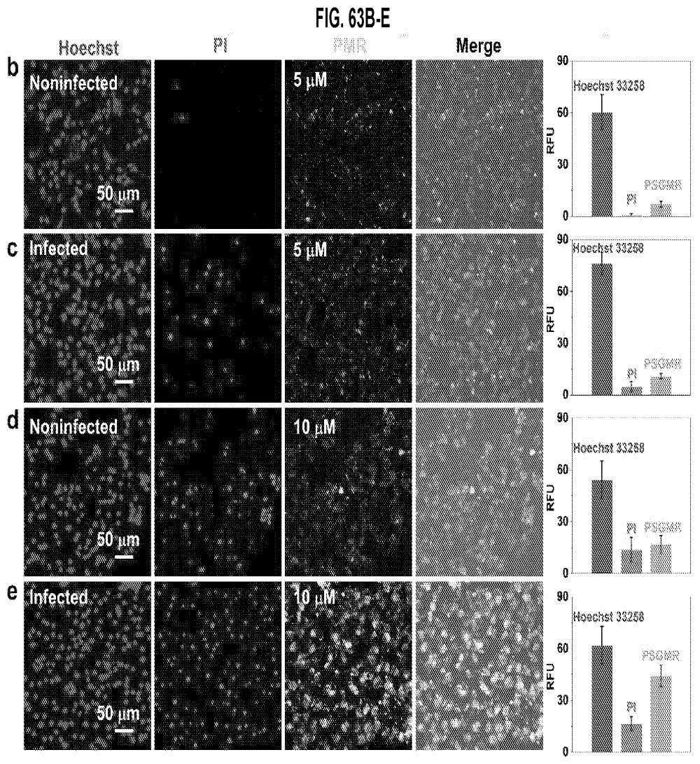

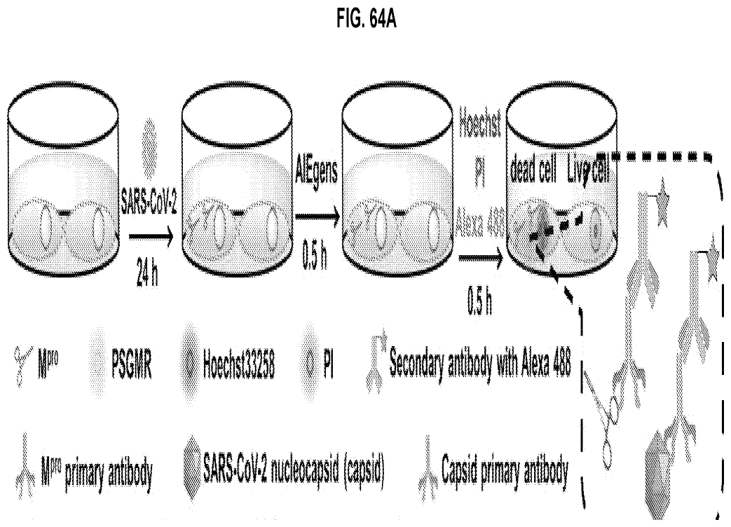

FIG. 64, or Figure S28 of Example 4, illustrates CLSM images and average fluorescence intensities of SARS-CoV-2 infected TMPRSS2-Vero cells incubation with HOECHST 33258™, PI, PSGMR, anti-SARS-CoV-2 nucleocapsid (Capsid) primary antibody, anti-SARS-CoV-2 M^{pro} primary antibody, and ALEXAFLUOR 488™-labeled secondary antibody (ALEXA 488™). (a) The experimental scheme. Cell images and average fluorescence intensities of SARS-CoV-2 infected TMPRSS2-Vero cells incubated with 5 μM PSGMR, (b) anti-SARS-CoV-2 Capsid primary antibody/Alexa 488, and (c) anti-SARS-CoV-2 M^{pro} primary antibody/Alexa 488. Scale bar=20 μm.

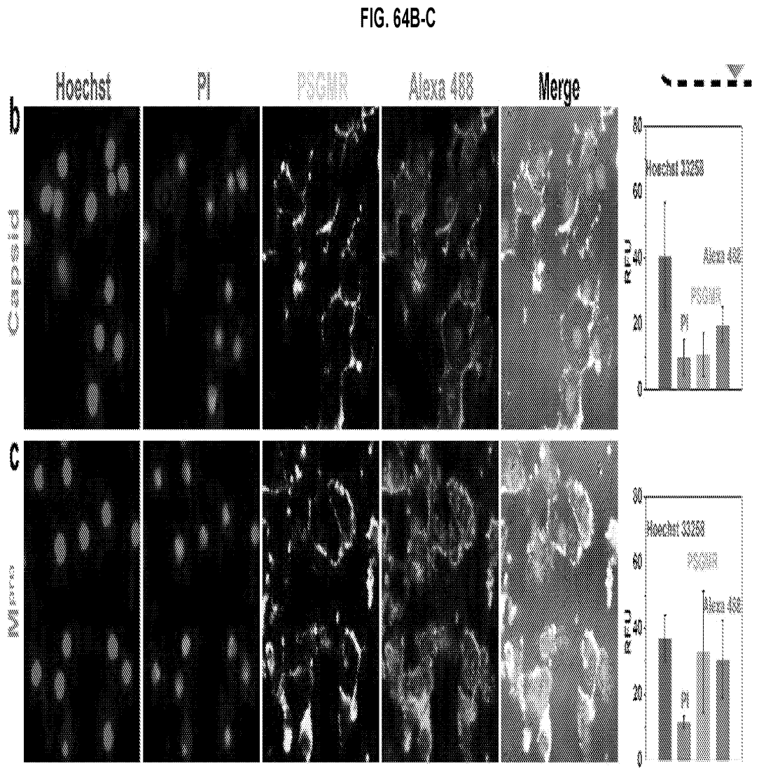

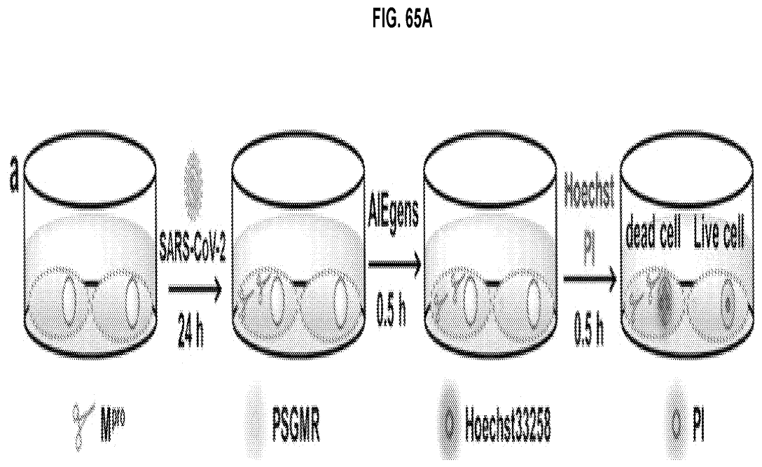

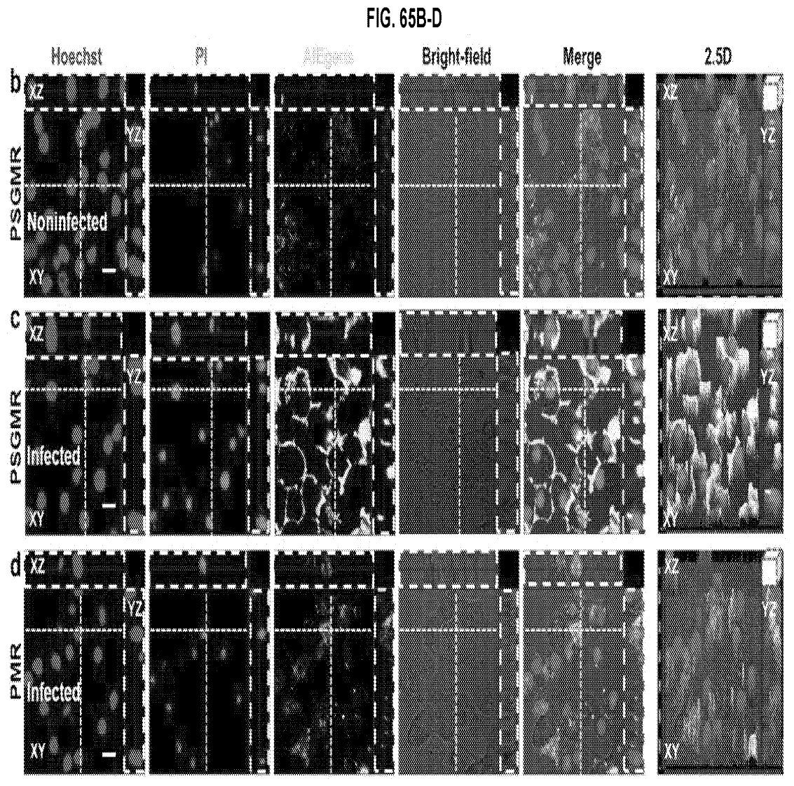

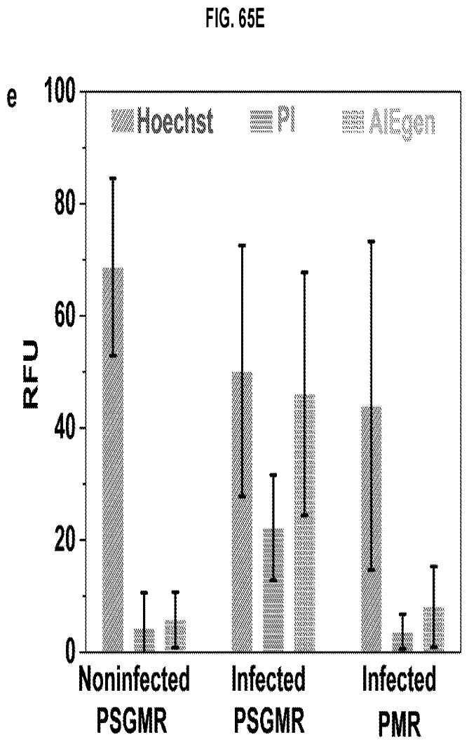

FIG. 65, or Figure S29 of Example 4, illustrates ARS-CoV-2 infected TMPRSS2-Vero cells incubation with HOECHST 33258™, PI, PSGMR, and PMR. (a) The experimental scheme. (b) Noninfected cells incubated with PSGMR. (c) SARS-CoV-2 infected cells incubation with PSGMR. (d) SARS-CoV-2 infected cells incubation with PMR. (e) The average fluorescence intensities of TMPRSS2-Vero cells incubated with Hoechst 33258, PI, PSGMR, and PMR. Scale bar=20 μm.

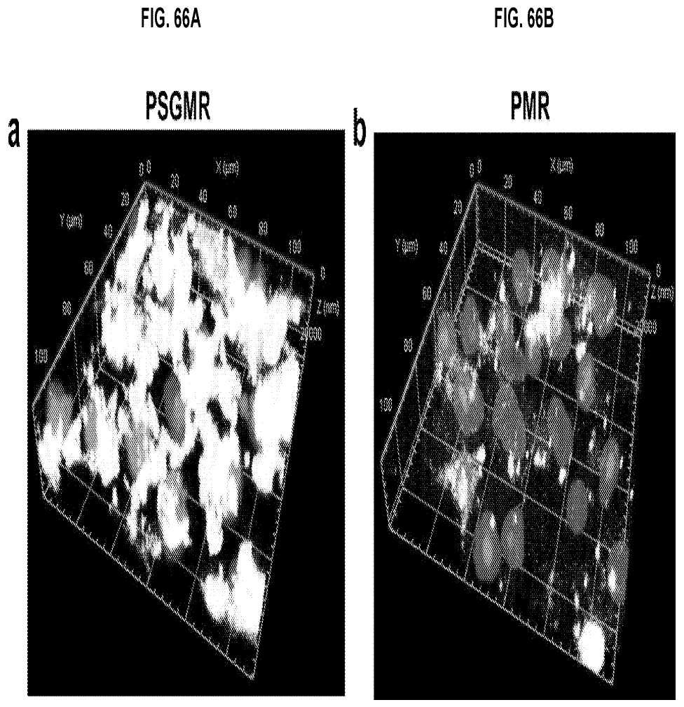

FIG. 66, or Figure S30 of Example 4, illustrates images corresponding to the three-dimensional map of SARS-CoV-2 infected TMPRSS2-Vero cells incubated with 5 M of (a) PSGMR and (b) PMR for 1 h.

### REFERENCES FOR EXAMPLE 1

1. Vaarala, M. H.; Porvari, K. S.; Kellokumpu, S.; KylloEnen, A. P.; Vihko, P. T., Expression of transmembrane serine protease TMPRSS2 in mouse and human tissues. J. Pathol. 2001, 193 (1), 134-140.
2. Bao, R.; Hernandez, K.; Huang, L.; Luke, J. J., ACE2 and TMPRSS2 expression by clinical, HLA, immune, and microbial correlates across 34 human cancers and matched normal tissues: implications for SARS-CoV-2 COVID-19. J Immunother Cancer 2020, 8 (2), e001020.
3. Sungnak, W.; Huang, N.; Becavin, C.; Berg, M.; Queen, R.; Litvinukova, M.; Talavera-Lopez, C.; Maatz, H.; Reichart, D.; Sampaziotis, F.; Worlock, K. B.; Yoshida, M.; Barnes, J. L.; Network, H. C. A. L. B., SARS-CoV-2 entry factors are highly expressed in nasal epithelial cells together with innate immune genes. Nat Med 2020, 26 (5), 681-687.
4. Cui, D.; et al., Single-cell RNA expression profiling of SARS-CoV-2-related ACE2 and TMPRSS2 in human trophectoderm and placenta. Ultrasound Obstet Gynecol 2021, 57 (2), 248-256.
5. Lukassen, S.; et al., SARS-CoV-2 receptor ACE2 and TMPRSS2 are primarily expressed in bronchial transient secretory cells. EMBO J 2020, 39 (10), e105114.
6. Tanabe, L. M.; List, K., The role of type II transmembrane serine protease-mediated signaling in cancer. FEBS J 2017, 284 (10), 1421-1436.
7. Epstein, R. J., The secret identities of TMPRSS2: Fertility factor, virus trafficker, inflammation moderator, prostate protector and tumor suppressor. Tumour Biol 2021, 43 (1), 159-176.
8. Lam, D. K.; Dang, D.; Flynn, A. N.; Hardt, M.; Schmidt, B. L., TMPRSS2, a novel membrane-anchored mediator in cancer pain. Pain 2015, 156 (5), 923-930.
9. Abbasi, A. Z.; Kiyani, D. A.; Hamid, S. M.; Saalim, M.; Fahim, A.; Jalal, N., Spiking dependence of SARS-CoV-2 pathogenicity on TMPRSS2. J Med Virol 2021, 93 (7), 4205-4218.
10. Hu, X.; et al., Discovery of TMPRSS2 Inhibitors from Virtual Screening as a Potential Treatment of COVID-19. ACS Pharmacol Transl Sci 2021, 4 (3), 1124-1135.
11. Fraser, B. J.; et al., Structure and activity of human TMPRSS2 protease implicated in SARS-CoV-2 activation. Nat Chem Biol 2022, 18 (9), 963-971.
12. Cheng, Z.; Zhou, J.; To, K. K.; Chu, H.; Li, C.; Wang, D.; Yang, D.; Zheng, S.; Hao, K.; Bosse, Y.; Obeidat, M.; Brandsma, C. A.; Song, Y. Q.; Chen, Y.; Zheng, B. J.; Li, L.; Yuen, K. Y., Identification of TMPRSS2 as a Susceptibility Gene for Severe 2009 Pandemic A(H1N1) Influenza and A(H7N9) Influenza. J Infect Dis 2015, 212 (8), 1214-1221.
13. Esumi, M.; Ishibashi, M.; Yamaguchi, H.; Nakajima, S.; Tai, Y.; Kikuta, S.; Sugitani, M.; Takayama, T.; Tahara, M.; Takeda, M.; Wakita, T., Transmembrane serine protease TMPRSS2 activates hepatitis C virus infection. Hepatology 2015, 61 (2), 437-446.
14. Iwata-Yoshikawa, N.; et al., Acute Respiratory Infection in Human Dipeptidyl Peptidase 4-Transgenic Mice Infected with Middle East Respiratory Syndrome Coronavirus. J Virol 2019, 93 (6), e01815-18.
15. Hoffmann, M.; et al., SARS-CoV-2 Cell Entry Depends on ACE2 and TMPRSS2 and Is Blocked by a Clinically Proven Protease Inhibitor. Cell 2020, 181 (2), 271-280.
16. Shang, J.; Wan, Y.; Luo, C.; Ye, G.; Geng, Q.; Auerbach, A.; Li, F., Cell entry mechanisms of SARS-CoV-2. Proc Natl Acad Sci USA 2020, 117 (21), 11727-11734.
17. Zang, R.; Castro, M. F. G.; McCune, B. T.; Zeng, Q.; Rothlauf, P. W.; Sonnek, N. M.; Liu, Z.; et al., Altered TMPRSS2 usage by SARS-CoV-2 Omicron impacts infectivity and fusogenicity. Nature 2022, 603 (7902), 706-714.
19. Padmanabhan, P.; Desikan, R.; Dixit, N. M., Targeting TMPRSS2 and Cathepsin B/L together may be synergistic against SARS-CoV-2 infection. PLoS Comput Biol 2020, 16 (12), e1008461.
20. Mollica, V.; Rizzo, A.; Massari, F., The pivotal role of TMPRSS2 in coronavirus disease 2019 and prostate cancer. Future Oncol. 2020, 16 (27), 2029-2033.
21. Cannalire, R.; Stefanelli, I.; Cerchia, C.; Beccari, A. R.; Pelliccia, S.; Summa, V., SARS-CoV-2 Entry Inhibitors: Small Molecules and Peptides Targeting Virus or Host Cells. Int J Mol Sci 2020, 21 (16), 5707.
22. Strope, J. D.; Pharm, D. C.; Figg, W. D., TMPRSS2: Potential Biomarker for COVID-19 Outcomes. J Clin Pharmacol 2020, 60 (7), 801-807.
23. Hempel, T.; Raich, L.; Olsson, S.; Azouz, N. P.; Klingler, A. M.; Hoffmann, M.; Pohlmann, S.; Rothenberg, M. E.; Noe, F., Molecular mechanism of inhibiting the SARS-CoV-2 cell entry facilitator TMPRSS2 with camostat and nafamostat. Chem Sci 2021, 12 (3), 983-992.
24. Ko, C. J.; et al., Inhibition of TMPRSS2 by HAI-2 reduces prostate cancer cell invasion and metastasis. Oncogene 2020, 39 (37), 5950-5963.
25. Zhang, F.; et al., SARS-CoV-2 pseudovirus infectivity and expression of viral entry-related factors ACE2, TMPRSS2, Kim-1, and NRP-1 in human cells from the respiratory, urinary, digestive, reproductive, and immune systems. J Med Virol 2021, 93 (12), 6671-6685.
26. Koch, J.; Uckeley, Z. M.; Doldan, P.; Stanifer, M.; Boulant, S.; Lozach, P. Y., TMPRSS2 expression dictates the entry route used by SARS-CoV-2 to infect host cells. EMBO J 2021, 40 (16), e107821.
27. Sun, Y. J.; Velez, G.; Parsons, D. E.; Li, K.; Ortiz, M. E.; Sharma, S.; McCray, P. B., Jr.; Bassuk, A. G.; Mahajan, V. B., Structure-based phylogeny identifies avoralstat as a TMPRSS2 inhibitor that prevents SARS-CoV-2 infection in mice. J Clin Invest 2021, 131 (10), e147973.
28. Lu, Q.; Nunez, E.; Lin, C.; Christensen, K.; Downs, T.; Carson, D. A.; Wang-Rodriguez, J.; Liu, Y. T., A sensitive array-based assay for identifying multiple TMPRSS2:ERG fusion gene variants. Nucleic Acids Res 2008, 36 (20), e130.
29. Shrimp, J. H.; Kales, S. C.; Sanderson, P. E.; Simeonov, A.; Shen, M.; Hall, M. D., An Enzymatic TMPRSS2 Assay for Assessment of Clinical Candidates and Discovery of Inhibitors as Potential Treatment of COVID-19. ACS Pharmacol Transl Sci 2020, 3 (5), 997-1007.
30. Chen, Y.; et al., A high-throughput screen for TMPRSS2 expression identifies FDA-approved compounds that can limit SARS-CoV-2 entry. Nat Commun 2021, 12 (1), 3907.
31. Li, F.; et al., Distinct mechanisms for TMPRSS2 expression explain organ-specific inhibition of SARS-CoV-2 infection by enzalutamide. Nat Commun 2021, 12 (1), 866.
32. Shapira, T.; et al., A TMPRSS2 inhibitor acts as a pan-SARS-CoV-2 prophylactic and therapeutic. Nature 2022, 605 (7909), 340-348.
33. McCarthy, J. R.; Weissleder, R., Multifunctional magnetic nanoparticles for targeted imaging and therapy. Adv Drug Deliv Rev 2008, 60 (11), 1241-1251.
34. Cheng, J. X.; Xie, X. S., Vibrational spectroscopic imaging of living systems: An emerging platform for biology and medicine. Science 2015, 350 (6264), aaa8870.
35. He, X. P.; Zang, Y.; James, T. D.; Li, J.; Chen, G. R., Probing disease-related proteins with fluorogenic composite materials. Chem Soc Rev 2015, 44 (13), 4239-4248.
36. Zhao, W. W.; Xu, J. J.; Chen, H. Y., Photoelectrochemical bioanalysis: the state of the art. Chem Soc Rev 2015, 44 (3), 729-741.
37. Labib, M.; Sargent, E. H.; Kelley, S. O., Electrochemical Methods for the Analysis of Clinically Relevant Biomolecules. Chem Rev 2016, 116 (16), 9001-9090.
38. Omar, M.; Aguirre, J.; Ntziachristos, V., Optoacoustic mesoscopy for biomedicine. Nat Biomed Eng 2019, 3 (5), 354-370.
39. Jiang, T.; Olson, E. S.; Nguyen, Q. T.; Roy, M.; Jennings, P. A.; Tsien, R. Y., Tumor imaging by means of proteolytic activation of cell-penetrating peptides. Proc Natl Acad Sci USA 2004, 101 (51), 17867-17872.
40. Olson, E. S.; Jiang, T.; Aguilera, T. A.; Nguyen, Q. T.; Ellies, L. G.; Scadeng, M.; Tsien, R. Y., Activatable cell penetrating peptides linked to nanoparticles as dual probes for in vivo fluorescence and MR imaging of proteases. Proc Natl Acad Sci USA 2010, 107 (9), 4311-4316.
41. Hong, G.; Antaris, A. L.; Dai, H., Near-infrared fluorophores for biomedical imaging. Nature Biomedical Engineering 2017, 1 (1), 0010.
42. Guo, Y.; Li, D.; Zhang, S.; Yang, Y.; Liu, J. J.; Wang, X.; Liu, C.; Milkie, D. E.; Moore, R. P.; Tulu, U. S.; Kiehart, D. P.; Hu, J.; Lippincott-Schwartz, J.; Betzig, E.; Li, D., Visualizing Intracellular Organelle and Cytoskeletal Interactions at Nanoscale Resolution on Millisecond Timescales. Cell 2018, 175 (5), 1430-1442.
43. Nguyen, S. S.; Prescher, J. A., Developing bioorthogonal probes to span a spectrum of reactivities. Nat Rev Chem 2020, 4, 476-489.
44. Yin, J.; Huang, L.; Wu, L.; Li, J.; James, T. D.; Lin, W., Small molecule based fluorescent chemosensors for imaging the microenvironment within specific cellular regions. Chem Soc Rev 2021, 50 (21), 12098-12150.
45. Liu, H. W.; Chen, L.; Xu, C.; Li, Z.; Zhang, H.; Zhang, X. B.; Tan, W., Recent progresses in small-molecule enzymatic fluorescent probes for cancer imaging. Chem Soc Rev 2018, 47 (18), 7140-7180.
46. Cheng, Y.; Borum, R. M.; Clark, A. E.; Jin, Z.; Moore, C.; Fajtova, P.; O'Donoghue, A. J.; Carlin, A. F.; Jokerst, J. V., A Dual-Color Fluorescent Probe Allows Simultaneous Imaging of Main and Papain-like Proteases of SARS-CoV-2-Infected Cells for Accurate Detection and Rapid Inhibitor Screening. Angew Chem Int Ed Engl 2022, 61 (9), e202113617.
47. Cheng, Y.; et al., Protease-Responsive Prodrug with Aggregation-Induced Emission Probe for Controlled Drug Delivery and Drug Release Tracking in Living Cells. Anal Chem 2016, 88 (17), 8913-8919.
48. Dai, J.; Cheng, Y.; Wu, J.; Wang, Q.; Wang, W.; Yang, J.; Zhao, Z.; Lou, X.; Xia, F.; Wang, S.; Tang, B. Z., Modular Peptide Probe for Pre/Intra/Postoperative Therapeutic to Reduce Recurrence in Ovarian Cancer. ACS Nano 2020, 14 (11), 14698-14714.
49. Zhang, H.; et al., Aggregate Science: From Structures to Properties. Adv Mater 2020, 32 (36), e2001457.
50. Yang, J.; et al., Tumor-Triggered Disassembly of a Multiple-Agent-Therapy Probe for Efficient Cellular Internalization. Angew Chem Int Ed Engl 2020, 59 (46), 20405-20410.
51. Feng, G.; Liu, B., Aggregation-Induced Emission (AIE) Dots: Emerging Theranostic Nanolights. Accounts of Chemical Research 2018, 51 (6), 1404-1414.
52. Mei, J.; Leung, N. L.; Kwok, R. T.; Lam, J. W.; Tang, B. Z., Aggregation-Induced Emission: Together We Shine, United We Soar! Chem Rev 2015, 115 (21), 11718-11940.
53. Cheng, Y.; et al., A Multifunctional Peptide-Conjugated AIEgen for Efficient and Sequential Targeted Gene Delivery into the Nucleus. Angew Chem Int Ed Engl 2019, 58 (15), 5049-5053.
54. Cheng, Y.; et al., Protease-Responsive Peptide-Conjugated Mitochondrial-Targeting AIEgens for Selective Imaging and Inhibition of SARS-CoV-2-Infected Cells. ACS Nano 2022, 16, 12305-12317.
55. Cheng, Y.; Sun, C.; Ou, X.; Liu, B.; Lou, X.; Xia, F., Dual-targeted peptide-conjugated multifunctional fluorescent probe with AIEgen for efficient nucleus-specific imaging and long-term tracing of cancer cells. Chem Sci 2017, 8 (6), 4571-4578.
56. Xia, F.; Wu, J.; Wu, X.; Hu, Q.; Dai, J.; Lou, X., Modular Design of Peptide- or DNA-Modified AIEgen Probes for Biosensing Applications. Acc Chem Res 2019, 52 (11), 3064-3074.
57. Yuan, Q.; Cheng, Y.; Lou, X.; Xia, F., Rational Fabrication and Biomedical Application of Biomolecule—Conjugated AIEgens through Click Reaction. Chinese Journal of Chemistry 2019, 37 (10), 1072-1082.
58. Levi, J.; Kothapalli, S. R.; Ma, T. J.; Hartman, K.; Khuri-Yakub, B. T.; Gambhir, S. S., Design, Synthesis, and Imaging of an Activatable Photoacoustic Probe. J. Am. Chem. Soc. 2010, 132 (32), 11264-11269.
59. Weinstain, R.; Savariar, E. N.; Felsen, C. N.; Tsien, R. Y., In vivo targeting of hydrogen peroxide by activatable cell-penetrating peptides. J Am Chem Soc 2014, 136 (3), 874-877.
60. Chen, X.; et al., Fusion protein linkers: property, design and functionality. Adv Drug Deliv Rev 2013, 65 (10), 1357-69.
61. Reddy Chichili, V. P.; Kumar, V.; Sivaraman, J., Linkers in the structural biology of protein-protein interactions. Protein Sci 2013, 22 (2), 153-67.
62. Hoffmann, M.; Hofmann-Winkler, H.; Smith, J. C.; Kruger, N.; Arora, P.; Sorensen, L. K.; et al., Camostat mesylate inhibits SARS-CoV-2 activation by TMPRSS2-related proteases and its metabolite GBPA exerts antiviral activity. EBioMedicine 2021, 65, 103255.

### REFERENCES FOR EXAMPLE 2

1. Cheng, Y. et al. Protease-Responsive Prodrug with Aggregation-Induced Emission Probe for Controlled Drug Delivery and Drug Release Tracking in Living Cells. Anal Chem 88, 8913-8919 (2016).
2. Cheng, Y. et al. A Multifunctional Peptide-Conjugated AIEgen for Efficient and Sequential Targeted Gene Delivery into the Nucleus. Angew Chem Int Ed Engl 58, 5049-5053 (2019).

### REFERENCES FOR EXAMPLE 3

1. Lu, R.; et al., Genomic characterisation and epidemiology of 2019 novel corona-virus: implications for virus origins and receptor binding. The Lancet 2020, 395 (10224), 565-574.
2. Cai, Y.; et al., Structural basis for enhanced infectivity and immune evasion of SARS-CoV-2 variants. Science 2021, 373 (6555), 642-648.
3. Kevadiya, B. D.; et al., Diagnostics for SARS-CoV-2 infections. Nat. Mater. 2021, 20 (5), 593-605.
4. Shin, M. D.; et al., COVID-19 vaccine development and a potential nanomaterial path forward. Nat. Nanotechnol. 2020, 15 (8), 646-655.
5. Iwasaki, A.; Omer, S. B., Why and How Vaccines Work. Cell 2020, 183 (2), 290-295.
6. Chauhan, G.; et al., Nanotechnology for COVID-19: Therapeutics and Vaccine Research. ACS. Nano. 2020, 14 (7), 7760-7782.
7. Ai, X.; et al., Surface Glycan Modification of Cellular Nanosponges to Promote SARS-CoV-2 Inhibition. J. Am. Chem. Soc. 2021, 143 (42), 17615-17621.
8. Xia, S.; et al., Inhibition of SARS-CoV-2 (previously 2019-nCoV) infection by a highly potent pan-coronavirus fusion inhibitor targeting its spike protein that harbors a high capacity to mediate membrane fusion. Cell. Res. 2020, 30 (4), 343-355.
9. Hoffmann, M.; et al., SARS-CoV-2 Cell Entry Depends on ACE2 and TMPRSS2 and Is Blocked by a Clinically Proven Protease Inhibitor. Cell 2020, 181 (2), 271-280.
10. Avanzato, V. A.; et al., Case Study: Prolonged Infectious SARS-CoV-2 Shedding from an Asymptomatic Immunocompromised Individual with Cancer. Cell 2020, 183 (7), 1901-1912.
11. Fedry, J.; et al., Structural insights into the cross-neutralization of SARS-CoV and SARS-CoV-2 by the human monoclonal antibody 47D11. Sci. Adv. 2021, 7, eabf5632.
12. Zhao, H.; et al., Cross-linking peptide and repurposed drugs inhibit both entry pathways of SARS-CoV-2. Nat Commun 2021, 12 (1), 1517.
13. Meyer, B.; et al.; Emmott, E., Characterising proteolysis during SARS-CoV-2 infection identifies viral cleavage sites and cellular targets with therapeutic potential. Nat. Commun. 2021, 12 (1), 5553.
14. Arafet, K.; et al., Mechanism of inhibition of SARS-CoV-2 M(pro) by N3 peptidyl Michael acceptor explained by QM/MM simulations and design of new derivatives with tuna-ble chemical reactivity. Chem. Sci. 2020, 12 (4), 1433-1444.
15. Zhang, L.; et al., Water-Triggered, Irreversible Conformational Change of SARS-CoV-2 Main Protease on Passing from the Solid State to Aqueous Solu-tion. J. Am. Chem. Soc. 2021, 143 (33), 12930-12934.
17. Iketani, S.; et al., Lead compounds for the development of SARS-CoV-2 3CL pro-tease inhibitors. Nat. Commun. 2021, 12, 2016.
18. Froggatt, H. M.; Heaton, B. E.; Heaton, N. S., Development of a Fluorescence-Based, High-Throughput SARS-CoV-2 3CLpro Re-porter Assay. J. Virol. 2020, 94 (22), e01265-20.
19. Cheng, Y.; et al., A Dual-Color Flu-orescent Probe Allows Simultaneous Imaging of Main and Papain-like Proteases of SARS-CoV-2-Infected Cells for Accurate Detec-tion and Rapid Inhibitor Screening. Angew. Chem. Int. Ed. 2021, e202113617.
20. Jin, Z.; et al., A Charge-Switchable Zwitterionic Peptide for Rapid Detection of SARS-CoV-2 Main Protease. Angew. Chem. Int. Ed. 2021, e202112995.
21. Yang, Z. M.; Xu, K. M.; Guo, Z. F.; Guo, Z. H.; Xu, B., Intracellu-lar Enzymatic Formation of Nanofibers Results in Hydrogelation and Regulated Cell Death. Adv. Mater. 2007, 19 (20), 3152-3156.
22. Zhou, J.; Xu, B., Enzyme-instructed self-assembly: a multistep process for potential cancer therapy. Bioconjug. Chem. 2015, 26 (6), 987-999.
23. Feng, Z.; et al., Self-Assembling Ability Determines the Activity of Enzyme-Instructed Self-Assembly for Inhibiting Cancer Cells. J. Am. Chem. Soc. 2017, 139 (43), 15377-15384.
24. Wang, H.; Feng, Z.; Xu, B., Instructed Assembly as Context-Dependent Signaling for the Death and Morphogenesis of Cells. Angew. Chem. Int. Ed. 2019, 58 (17), 5567-5571.
25. He, H.; Tan, W.; Guo, J.; Yi, M.; Shy, A. N.; Xu, B., Enzymatic Noncovalent Synthesis. Chem. Rev. 2020, 120 (18), 9994-10078.
26. Feng, Z.; et al., Enzymatic Assemblies Disrupt the Membrane and Target Endoplasmic Reticulum for Selective Cancer Cell Death. J. Am. Chem. Soc. 2018, 140 (30), 9566-9573.
27. Kumar, S.; et al., Peptidomimetic-Based Multidomain Targeting Offers Critical Evaluation of Abeta Structure and Toxic Function. J. Am. Chem. Soc. 2018, 140 (21), 6562-6574.
28. Feng, Z.; et al., Supramolecular catalysis and dynamic assemblies for medicine. Chem. Soc. Rev. 2017, 46 (21), 6470-6479.
29. Yang, Z.; Liang, G.; Guo, Z.; Guo, Z.; Xu, B., Intracellular hydro-gelation of small molecules inhibits bacterial growth. Angew. Chem. Int. Ed. 2007, 46 (43), 8216-8219.
30. Cheng, Y.; Dai, J.; Sun, C.; Liu, R.; Zhai, T.; Lou, X.; Xia, F., An Intracellular H2 O2-Responsive AIEgen for the Peroxidase-Mediated Selective Imaging and Inhibition of Inflammatory Cells. Angew. Chem. Int. Ed. 2018, 57 (12), 3123-3127.
31. Gao, Y.; Shi, J.; Yuan, D.; Xu, B., Imaging enzyme-triggered self-assembly of small molecules inside live cells. Nat. Commun. 2012, 3, 1033.
32. He, H.; et al., Enzymatic Cleavage of Branched Peptides for Targeting Mito-chondria. J. Am. Chem. Soc. 2018, 140 (4), 1215-1218.
33. Zhang, X. H.; et al., Photothermal-Promoted Mor-phology Transformation In vivo Monitored by Photoacoustic Imaging. Nano. Lett. 2020, 20 (2), 1286-1295.
34. He, H.; Guo, J.; Xu, J.; Wang, J.; Liu, S.; Xu, B., Dynamic Contin-uum of Nanoscale Peptide Assemblies Facilitates Endocytosis and Endosomal Escape. Nano. Lett. 2021, 21 (9), 4078-4085.
35. Wu, D.; et al., Fluorescent chemosensors: the past, present and future. Chem. Soc. Rev. 2017, 46 (23), 7105-7123.
36. Pascal, S.; David, S.; Andraud, C.; Maury, O., Near-infrared dyes for two-photon absorption in the short-wavelength infrared: strategies towards optical power limiting. Chem. Soc. Rev. 2021, 50 (11), 6613-6658.
37. Han, A.; et al., Peptide-Induced AIEgen Self-Assembly: A New Strategy to Realize Highly Sensitive Fluorescent Light-Up Probes. Anal. Chem. 2016, 88 (7), 3872-3878.
38. Zhao, Y.; et al., Spatiotemporally Controllable Peptide-Based Nanoas-sembly in Single Living Cells for a Biological Self-Portrait. Adv. Mater. 2017, 29 (32), 1601128.
39. Li, J.; Fang, Y.; Zhang, Y.; Wang, H.; Yang, Z.; Ding, D., Supra-molecular Self-Assembly-Facilitated Aggregation of Tumor-Specific Transmembrane Receptors for Signaling Activation and Converting Immunologically Cold to Hot Tumors. Adv. Mater. 2021, 33 (16), e2008518.
40. Kwok, R. T.; Leung, C. W.; Lam, J. W.; Tang, B. Z., Biosensing by luminogens with aggregation-induced emission characteris-tics. Chem. Soc. Rev. 2015, 44 (13), 4228-4238.
41. Mei, J.; et al., Aggre-gation-Induced Emission: Together We Shine, United We Soar! Chem. Rev. 2015, 115 (21), 11718-11940.
42. Shi, H.; Liu, J.; Geng, J.; Tang, B. Z.; Liu, B., Specific detection of integrin alphavbeta3 by light-up bioprobe with aggregation-induced emission characteristics. J. Am. Chem. Soc. 2012, 134 (23), 9569-9572.
43. Yuan, Y.; Zhang, C. J.; Gao, M.; Zhang, R.; Tang, B. Z.; Liu, B., Specific light-up bioprobe with aggregation-induced emission and activatable photoactivity for the targeted and image-guided pho-todynamic ablation of cancer cells. Angew. Chem. Int. Ed. 2015, 54 (6), 1780-1786.
44. Cheng, Y.; et al., A Multifunctional Peptide-Conjugated AIEgen for Efficient and Sequential Targeted Gene Delivery into the Nucleus. Angew. Chem. Int. Ed. 2019, 58 (15), 5049-5053.
45. Yang, J.; et al., Tumor-Triggered Disassembly of a Multiple-Agent-Therapy Probe for Efficient Cellular Internalization. Angew. Chem. Int. Ed. 2020, 59 (46), 20405-20410.
46. Cheng, Y.; et al., Dual-targeted peptide-conjugated multifunctional fluorescent probe with AIEg-en for efficient nucleus-specific imaging and long-term tracing of cancer cells. Chem. Sci. 2017, 8 (6), 4571-4578.
47. Lowe, T. L.; et al., Struc-ture-Function Relationships for Inhibitors of â-Amyloid Toxicity Containing the Recognition Sequence KLVFF. Biochemistry. 2001, 40, 7882-7889.
48. Cheng, D. B.; et al., Endogenous Reactive Oxygen Spe-cies-Triggered Morphology Transformation for Enhanced Cooper-ative Interaction with Mitochondria. J. Am. Chem. Soc. 2019, 141 (18), 7235-7239.
49. Zhang, L.; Jing, D.; Jiang, N.; Rojalin, T.; Baehr, C. M.; Zhang, D.; Xiao, W.; Wu, Y.; Cong, Z.; Li, J. J.; Li, Y.; Wang, L.; Lam, K. S., Transformable peptide nanoparticles arrest HER2 signaling and cause cancer cell death in vivo. Nat. Nanotechnol. 2020, 15 (2), 145-153.
50. Chen, X.; Zaro, J. L.; Shen, W. C., Fusion protein linkers: prop-erty, design and functionality. Adv. Drug. Deliv. Rev. 2013, 65 (10), 1357-1369.
51. Reddy Chichili, V. P.; et al., Allosteric Inhibition of the SARS-CoV-2 Main Protease: Insights from Mass Spectrometry Based Assays. Angew. Chem. Int. Ed. 2020, 59 (52), 23544-23548.
53. Jin, Z.; et al., Structure of M(pro) from SARS-CoV-2 and discovery of its inhibitors. Nature 2020, 582 (7811), 289-293.
54. Futaki, S., Membrane-permeable arginine-rich peptides and the translocation mechanisms. Adv. Drug. Deliv. Rev. 2005, 57 (4), 547-558.
55. Perret, F.; et al., Anionic Fullerenes, Ca-lixarenes, Coronenes, and Pyrenes as Activators of Oli-go/Polyarginines in Model Membranes and Live Cells. J. Am. Chem. Soc. 2005, 127 (4), 1114-1115.
56. Wender, P. A.; Galliher, W. C.; Goun, E. A.; Jones, L. R.; Pillow, T. H., The design of guanidinium-rich transporters and their in-ternalization mechanisms. Adv. Drug. Deliv. Rev. 2008, 60 (4-5), 452-472.
57. Peraro, L.; Kritzer, J. A., Emerging Methods and Design Princi-ples for Cell-Penetrant Peptides. Angew. Chem. Int. Ed. 2018, 57 (37), 11868-11881.
58. Hamley, I. W.; Krysmann, M. J., Effect of PEG Crystallization on the Self-Assembly of PEG/Peptide Copolymers Containing Amyloid Peptide Fragments. Langmuir. 2008, 24 (15), 8210-8214.
59. Qu, A.; Huang, F.; Li, A.; Yang, H.; Zhou, H.; Long, J.; Shi, L., The synergistic effect between KLVFF and self-assembly chaperones on both disaggregation of beta-amyloid fibrils and reducing con-sequent toxicity. Chem. Commun. 2017, 53 (7), 1289-1292.
60. Lv, H.; et al., Toxicity of cationic lipids and cationic polymers in gene delivery. J. Control. Release. 2006, 114 (1), 100-109.
61. Grimaldi, N.; et al., Lipid-based nanovesicles for nanomed-icine. Chem. Soc. Rev. 2016, 45 (23), 6520-6545.

### REFERENCES FOR EXAMPLE 4

1. Mellott, D. M. et al. A Clinical-Stage Cysteine Protease Inhibitor blocks SARS-CoV-2 Infection of Human and Monkey Cells. ACS Chem Biol 16, 642-650 (2021).
2. Froggatt, H. M., et al. Development of a Fluorescence-Based, High-Throughput SARS-CoV-2 3CLpro Reporter Assay. Journal of Virology 94, e01265-01220 (2020).
3. Penalver, L. et al. A Ligand Selection Strategy Identifies Chemical Probes Targeting the Proteases of SARS-CoV-2. Angew Chem Int Ed Engl 60, 6799-6806 (2021).
4. Cheng, Y. et al. Protease-Responsive Prodrug with Aggregation-Induced Emission Probe for Controlled Drug Delivery and Drug Release Tracking in Living Cells. Anal Chem 88, 8913-8919 (2016).
5. Cheng, Y. et al. A Multifunctional Peptide-Conjugated AIEgen for Efficient and Sequential Targeted Gene Delivery into the Nucleus. Angew Chem Int Ed Engl 58, 5049-5053 (2019).

A number of embodiments of the invention have been described. Nevertheless, it can be understood that various modifications may be made without departing from the spirit and scope of the invention. Accordingly, other embodiments are within the scope of the following claims.

1: A product of manufacture, or synthetic peptide or polypeptide, comprising:
(a) a protease-responsive and surface-potential-tunable peptide-conjugated AIEgens (EGTP) for TMPRSS2 selective imaging and accurate inhibitor screening, where EGTP comprises four segments: the first is a polyglutamic acid (Glu, E for short in EGTP) that increases the solubility, blocks the positive charges and cell-penetrating ability of PyTPE;
(b) a spacer trimylglycine (GGG, G) designed to enhance probe flexibility and reduce steric hindrance for TMPRSS2-substrate interactions;
(c) a TMPRSS2-responsive peptide (QAR, T), which can be cleaved by TMPRSS2 after QAR sequence; and
(d) a positive charged AIEgens (PyTPE, P).

2: A product of manufacture, or synthetic PSGMR (or (Pra)KLVFFGGGSAVLQ/SGFRKMAGGGRRRRRR) (SEQ ID NO:18) (the Mpro-responsive modular (self-assembling) peptide or polypeptide, comprising:
(a) an AIEgen (PyTPE, P for short in PSGMR) PyTPE has bright yellow fluorescence, excellent biocompatibility, and good photostability;
(b) a self-assembling peptide (KLVFF, S) β-sheet-forming peptide derived from A-amyloid protein, that can spontaneously self-assemble into amyloid fibrils through Tr-Tr stacking, hydrogen bonding, and hydrophobic interactions;
(c) a spacer trimylglycine (GGG, G) to enhance flexibility and reduce steric hindrance for Mpro-substrate interactions;
(d) a Mpro-responsive peptide (SAVLQ/SGFRKMA (SEQ ID NO: 6), M), and
(e) a positive hexamolyarginine (RRRRRR (SEQ ID NO:7), R) that increases both the solubility and cell-penetrating ability of PSGMR,
wherein (a) to (e) are covalently coupled.

3: A nanofiber comprising a plurality of synthetic PSGMR (or (Pra)KLVFFGGGSAVLQ/SGFRKMAGGGRRRRRR) (SEQ ID NO: 18) (the main protease (Mpro)-responsive modular (self-assembling) peptides or polypeptides of claim 2.

4: A pharmaceutical composition comprising a nanofiber of claim 3.

5: A method for selectively inhibiting the growth of a viral-infected (optionally SARS-CoV-2-infected) cell, or treating or preventing virus replication, optionally intracellular virus replication, or treating or preventing a viral infection in an individual in need thereof, comprising exposing the viral-infected infected cell or the virus to a nanofiber of claim 3.

6-7. (canceled)

8: A method for selectively inhibiting the growth of a viral-infected (optionally SARS-CoV-2-infected) cell, or treating or preventing virus replication, optionally intracellular virus replication, or treating or preventing a viral infection in an individual in need thereof, comprising administering to an individual in a pharmaceutical composition of claim 4.

9: The method of claim 2, wherein (a) to (e) are covalently coupled through a Fmoc-based solid-phase peptide synthesis and a copper-catalyzed azide-alkyne click reaction.

10: The method of claim 2, wherein the synthetic PSGMR forms loose nanoparticles due to the positive hexamolyarginine residues on the surface and hydrophobic core of PyTPE,

11: The method of claim 2, wherein after being cleaved by Mpro, the hydrophilic hexamolyarginine is separated from PSG, and the self-assembling peptides with one negative charge is exposed to the nanoparticle surface, resulting in increasing self-assembly and electrostatic attraction as well as the decreasing hydrophilicity leading to PSG aggregation and nanofiber formation with strong yellow fluorescence.
